(19) United States
(12) Patent Application Publication
(10) Pub. No.: US 2025/0304970 A1
(43) Pub. Date: Oct. 2, 2025

(54) Methods for Reducing Ataxin-2 Expression

(71) Applicant: Ionis Pharmaceuticals, Inc., Carlsbad, CA (US)

(72) Inventor: Frank Rigo, Carlsbad, CA (US)

(73) Assignee: Ionis Pharmaceuticals, Inc., Carlsbad, CA (US)

(21) Appl. No.: 18/956,812

(22) Filed: Nov. 22, 2024

Related U.S. Application Data
(63) Continuation of application No. 17/323,826, filed on May 18, 2021, now Pat. No. 12,188,020.
Application No. 17/323,826 is a continuation of application No. 16/063,852, filed on Jun. 19, 2018, now abandoned.
(60) Provisional application No. 62/273,689, filed on Dec. 31, 2015.

(51) Int. Cl. C12N 15/113; A61K 31/7088; A61P 25/28; C12N 5/0793

(52) U.S. Cl. CPC C12N 15/1138; A61K 31/7088; A61P 25/28; C12N 5/0619; C12N 15/113; A01K 2227/105; A01K 2267/0318; C12N 2310/11; C12N 2310/315; C12N 2310/3233; C12N 2510/00

(57) ABSTRACT

Provided herein are methods for decreasing Ataxin-2 mRNA expression. Such methods are useful to ameliorate symptoms of Ataxin-2 associated diseases. Such Ataxin-2 associated diseases include amyotrophic lateral sclerosis (ALS). Such symptoms include loss of motor function, reduced CMAP amplitude, denervation, and loss of motor neurons.

### CROSS-REFERENCE TO RELATED APPLICATIONS

This application is a Continuation of U.S. patent application Ser. No. 17/323,826, filed on May 18, 2021, which is a Continuation of U.S. patent application Ser. No. 16/063,852, filed on Jun. 19, 2018, now abandoned, which is National Stage Application under 35 U.S.C. 371 of PCT Application No. PCT/US2016/069406, filed Dec. 30, 2016, which claims the benefit of priority to U.S. Provisional Application No. 62/273,689, filed Dec. 31, 2015, each of which is incorporated by reference herein in its entirety for any purpose.

### SEQUENCE LISTING

The present application is being filed along with a Sequence Listing in electronic format. The Sequence Listing is provided as a file entitled BIOL0281SEQ.xml, created on Jul. 11, 2023, which is 479,868 bytes in size. The information in the electronic format of the sequence listing is incorporated herein by reference in its entirety.

### FIELD

Provided are methods for reducing expression of Ataxin-2 mRNA, and optionally reducing expression of Ataxin-2 protein, in an animal. Such methods are useful to prevent or ameliorate at least one symptom of a neurodegenerative disease. Such symptoms include loss of motor function, reduced CMAP amplitude, denervation, and loss of motor neurons. Such neurodegenerative diseases include spinocerebellar ataxia type 2 (SCA2), amyotrophic lateral sclerosis (ALS), and parkinsonism.

### BACKGROUND

Ataxin-2 is a protein encoded by the ATXN2 gene. Ataxin-2 is expressed systemically and is located in the cytoplasm. It is thought that Ataxin-2 interacts with the endoplasmic reticulum and is involved in the regulation of RNA stability. The ATXN2 gene includes a CAG trinucleotide repeat expansion. Normally, the CAG trinucleotide repeat is repeated approximately 22 times. CAG expansions of more than 22 repeats are associated with certain neurodegenerative diseases.

Spinocerebellar ataxia type 2 (SCA2) is an autosomal dominant neurodegenerative disease characterized by progressive functional and cell loss of neurons in the cerebellum, brain stem, and spinal cord. The cause of SCA2 is CAG expansion in the ATXN2 gene resulting in polyglutamine (polyQ) expansion in the ataxin-2 protein. Patients with SCA2 are characterized by progressive cerebellar ataxia, slow saccadic eye movements, and other neurologic features such as neuropathy (Pulst, S. M. (ed.), Genetics of Movement Disorders. Elsevier, Inc., Amsterdam, 2003, pp. 19-34.). Moderate CAG expansion in the ATXN2 gene is also associated with parkinsonism or amyotrophic lateral sclerosis (ALS) indistinguishable from the idiopathic forms of these diseases (Kim et al., Arch. Neurol., 2007, 64: 1510-1518; Ross et al., Hum. Mol. Genet., 2011, 20: 3207-3212; Corrado et al., Hum. Genet., 2011, 130: 575-580; Elden et al., Nature, 2010, 466: 1069-1075; Van Damme et al., Neurology, 2011, 76: 2066-2072).

The pathogenic functions of polyQ disease proteins that occur with polyQ expansion may be attributed to the gain of toxicity associated with the development of intranuclear inclusion bodies or with soluble toxic oligomers (Lajoie et al., PLoS One, 2011, 5: e15245). While SCA2 patient brains are characterized by loss of Purkinje cells, SCA2 Purkinje cells lack inclusion bodies indicating polyQ-expanded ataxin-2 may cause toxicity that is unrelated to inclusion body formation (Huynh et al., Ann. Neurol., 1999, 45: 232-241). Functions gained in polyQ-expanded ataxin-2 may include anomalous accumulation in Golgi bodies (Huynh et al., Hum. Mol. Genet., 2003, 12: 1485-1496), gain-of-normal functions (Duvick et al., Neuron, 2010, 67: 929-935), and sequestering of transcription factors (TFs) and glyceraldehyde-3-phosphate dehydrogenase like for other polyQ proteins (Yamanaka et al., Methods Mol. Biol., 2010: 648, 215-229; Koshy et al., Hum. Mol. Genet., 1996, 5: 1311-1318; Burke et al., Nat. Med., 1996, 2: 347-350). Some normal functions of ataxin-2 have been characterized. Ataxin-2 is present in stress granules and P-bodies suggesting functions in sequestering mRNAs and protein translation regulation during stress (Nonhoff et al., Mol. Biol. Cell, 2007, 18: 1385-1396). Ataxin-2 overexpression interfered with the P-body assembly, while underexpression interfered with stress granule assembly (Nonhoff et al., Mol. Biol. Cell, 2007, 18: 1385-1396). Interactions with polyA-binding protein 1, the RNA splicing factor A2BP1/Foxl and polyribosomes further support roles for ataxin-2 in RNA metabolism (Shibata et al., Hum. Mol. Genet., 2000, 9: 1303-1313; Ciosk et al., Development, 2004, 131: 4831-4841; Satterfield et al., Hum. Mol. Genet., 2006, 15: 2523-2532). Ataxin-2 is a regulator of EGF receptor internalization and signaling by the way of its interactions with SRC kinase and the endocytic protein CIN85 (Nonis et al., Cell Signal., 2008, 20: 1725-1739). Ataxin-2 also interacts with the ALS-related protein TDP-43 in an RNA-dependent manner and familial and sporadic ALS associates with the occurrence of long normal CAG repeat expansion ATXN2 (Elden et al., Nature, 2010, 466: 1069-1075; Van Damme et al., Neurology, 2011, 76: 2066-2072).

Currently there is a lack of acceptable options for treating neurodegenerative diseases including neurodegenerative diseases associated with a CAG expansion in Ataxin-2. It is therefore an object herein to provide methods for the treatment of such diseases.

### SUMMARY OF THE INVENTION

Provided herein are methods for reducing expression of Ataxin-2 mRNA, and optionally reducing the amount of Ataxin-2 protein, in an animal. In certain embodiments, compounds useful for modulating expression of Ataxin-2 mRNA are oligomeric compounds. In certain embodiments, the oligomeric compound comprises a modified oligonucleotide.

In certain embodiments, Ataxin-2 mRNA expression is reduced in a cell or tissues. In certain embodiments, the cell or tissue is in an animal. In certain embodiments, the animal is a human. In certain embodiments, the amount of Ataxin-2 protein is reduced. Such reduction can occur in a time-dependent manner or in a dose-dependent manner.

Also provided are methods useful for ameliorating at least one symptom of ALS. In certain embodiments, such symptoms include loss of motor function, reduced CMAP amplitude, denervation, and loss of motor neurons.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety. Definitions Unless specific definitions are provided, the nomenclature used in connection with, and the procedures and techniques of, analytical chemistry, synthetic organic chemistry, and medicinal and pharmaceutical chemistry described herein are those well known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

“Administering” means providing a pharmaceutical agent to an animal. “Administered prior to the detection of the at least one symptom” is prophylactic administration and means providing the pharmaceutical agent to an animal before a symptom of ALS is apparent through observation or clinical diagnosis.

“ALS” or “amyotrophic lateral sclerosis” means a progressive neurodegenerative disease that affects nerve cells in the brain and the spinal cord. ALS causes progressive degeneration of motor neurons, eventually resulting in their death. There are familial and sporadic forms of ALS. “At least one symptom of ALS” includes loss of motor function, reduced CMAP amplitude, denervation, and loss of motor neurons.

“Animal” means a human or non-human animal.

“Antisense activity” means any detectable and/or measurable change attributable to the hybridization of an oligomeric compound to its target nucleic acid. In certain embodiments, antisense activity is a decrease in the amount or expression of a target nucleic acid or protein encoded by such target nucleic acid compared to target nucleic acid levels or target protein levels in the absence of the oligomeric compound. In certain embodiments, antisense activity is a change in splicing of a pre-mRNA nucleic acid target. In certain embodiments, antisense activity is an increase in the amount or expression of a target nucleic acid or protein encoded by such target nucleic acid compared to target nucleic acid levels or target protein levels in the absence of the oligomeric compound.

“Ameliorate” or “amelioration” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom is loss of motor function, reduced CMAP amplitude, denervation, and loss of motor neurons. In certain embodiments, amelioration of these symptoms results in improved motor function, increased or stabilized CMAP amplitude, preservation or increase in innervation, and reduced motor neuron loss.

“Bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

“Conjugate group” means a group of atoms that is directly or indirectly attached to an oligonucleotide. Conjugate groups include a conjugate moiety and a conjugate linker that attaches the conjugate moiety to the oligonucleotide.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Duplex” means two oligomeric compounds that are paired. In certain embodiments, the two oligomeric compounds are paired via hybridization of complementary nucleobases.

“Gapmer” means an oligomeric compound comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Identifying an animal having ALS” means identifying an animal having been diagnosed with ALS or predisposed to develop ALS. Individuals predisposed to develop ALS include those having one or more risk factors for developing ALS include having a genetic predisposition for ALS including mutations in any of ATXN2, SOD1, C9ORF72, FUS, and TDP43. Diagnosis may be accomplished by any method including evaluating an individual's medical history or standard clinical tests or assessments, such as, electromyography (EMG), nerve conduction velocity (NCV), and magnetic resonance imaging (MRI), and genetic testing of ATXN2, SOD1, C9ORF72, FUS, and/or TDP43.

“Internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage (“phosphodiester internucleoside linkage”). Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage.

“Non-bicyclic sugar moiety” means a modified sugar moiety that comprises a modification, such as a substitutent, that does not form a bridge between two atoms of the sugar to form a second ring.

“MOE” means methoxyethyl. “2′-MOE” means a —OCH₂CH₂OCH₃ group at the 2′ position of a furanosyl ring.

“Nucleobase” means an unmodified nucleobase or a modified nucleobase. As used herein “an “unmodified nucleobase” is adenine (A), thymine (T), cytosine (C), uracil (U), and guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases in a nucleic acid or oligonucleotide independent of any sugar or internucleoside linkage modification.

“Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a continuous sequence (i.e. no additional nucleosides are present between those that are linked).

“Oligomeric compound” means a compound comprising an oligonucleotide and optionally one or more additional features, such as a conjugate group or terminal group.

“Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

“Reducing or inhibiting the expression or amount” refers to a reduction or blockade of the expression or amount relative to the expression or amount in an untreated or control sample and does not necessarily indicate a total elimination of expression or amount.

“Single-stranded” in reference to an oligomeric compound means such a compound that is not paired with a second oligomeric compound to form a duplex.

“Standard cell assay” means the assay described in Example 1 and reasonable variations thereof.

“Standard in vivo experiment” means the procedure described in Example 2 and reasonable variations thereof.

“Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. Modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Therapeutically effective amount” means an amount of a pharmaceutical agent that provides a therapeutic benefit to an animal. For example, a therapeutically effective amount improves a symptom of a disease.

The present disclosure provides the following non-limiting numbered embodiments:

Embodiment 1. A method comprising administering to an animal having ALS an oligomeric compound comprising a modified oligonucleotide, wherein the modified oligonucleotide consists of 12 to 30 linked nucleosides, and wherein the modified oligonucleotide has a nucleobase sequence that is complementary to an Ataxin-2 nucleic acid.

Embodiment 2. A method comprising identifying an animal having ALS and administering to the animal having ALS an oligomeric compound comprising a modified oligonucleotide, wherein the modified oligonucleotide consists of 12 to 30 linked nucleosides, and wherein the modified oligonucleotide has a nucleobase sequence that is complementary to the nucleobase sequence of an Ataxin-2 nucleic acid.

Embodiment 3. The method of embodiment 1 or 2, wherein the modified oligonucleotide has a nucleobase sequence that is at least 90% complementary to the Ataxin-2 nucleic acid.

Embodiment 4. The method of embodiment 1 or 2, wherein the modified oligonucleotide has a nucleobase sequence that is at least 95% complementary to the Ataxin-2 nucleic acid.

Embodiment 5. The method of embodiment 1 or 2, wherein the modified oligonucleotide has a nucleobase sequence that is 100% complementary to the Ataxin-2 nucleic acid.

Embodiment 6. The method of any of embodiments 1-5 wherein the administering results in amelioration of at least one symptom of ALS.

Embodiment 7. The method of any of embodiments 1-6 wherin the oligomeric compound is administered prior to the detection of the at least one symptom.

Embodiment 8. The method of embodiment 6 or 7, wherein the at least one symptom of ALS is loss of motor function, reduced CMAP amplitude, denervation, and loss of motor neurons.

Embodiment 9. The method of any of embodiments 6-8, wherein the amelioration is the slowing of progression of at least one symptom.

Embodiment 10. The method of any of embodiments 6-9, wherein the amelioration is the delay of onset of at least one symptom.

Embodiment 11. The method of any of embodiments 6-10, wherein the amelioration is the reduction of severity of at least one symptom.

Embodiment 12. The method of any of embodiments 6-11, wherein the amelioration is the reduction of frequency of at least one symptom.

Embodiment 13. The method of any of embodiments 1-12, wherein expression of Ataxin-2 mRNA is reduced in the animal.

Embodiment 14. The method of any of embodiments 1-13, wherein the amount of Ataxin-2 protein is reduced in the animal.

Embodiment 15. The method of any of embodiments 1-14, wherein the animal is a human.

Embodiment 16. The method of any of embodiments 1-15, wherein the nucleobase sequence of Ataxin-2 nucleic acid is SEQ ID NO: 3, the complement of SEQ ID NO: 4, or SEQ ID NO: 5.

Embodiment 17. The method of any of embodiments 1-16, wherein the oligomeric compound is single-stranded.

Embodiment 18. The method of any of embodiment 1-17, wherein the modified oligonucleotide comprises at least one modified nucleoside.

Embodiment 19. The method of embodiment 18, wherein the modified oligonucleotide comprises at least one modified nucleoside comprising a modified sugar moiety.

Embodiment 20. The method of embodiment 19, wherein the modified oligonucleotide comprises at least one modified nucleoside comprising a bicyclic sugar moiety.

Embodiment 21. The method of embodiment 20, wherein the modified oligonucleotide comprises at least one modified nucleoside comprising a bicyclic sugar moiety having a 2′-4′ bridge, wherein the 2-4′ bridge is selected from —O—CH₂—; —O—CH₂—CH₂; and —O—CH(CH₃)—.

Embodiment 22. The method of any of embodiments 18-21, wherein the modified oligonucleotide comprises at least one modified nucleoside comprising a modified non-bicyclic sugar moiety.

Embodiment 23. The method of embodiment 22, wherein the modified oligonucleotide comprises at least one modified nucleoside comprising a non-bicyclic sugar moiety comprising a 2′-MOE or 2′-OMe.

Embodiment 24. The method of any of embodiments 18-23, wherein the modified oligonucleotide comprises at least one modified nucleoside comprising a sugar surrogate.

Embodiment 25. The method of any of embodiments 1-24, wherein the modified oligonucleotide comprises at least one modified nucleoside comprising a sugar surrogate selected from a morpholino, a PNA, a F-HNA, a THP, or a modified THP.

Embodiment 26. The method of any of embodiments 1-25, wherein the modified oligonucleotide has a sugar motif comprising:
a 5′-region consisting of 1-5 linked 5′-nucleosides;
a central region consisting of 6-10 linked central region nucleosides; and
a 3′-region consisting of 1-5 linked 3′-region nucleosides; wherein each of the 5′-region nucleosides and each of the 3′-region nucleosides comprises a modified sugar moiety and each of the central region nucleosides comprises an unmodified DNA sugar moiety.

Embodiment 27. The method of any of embodiments 1-26, wherein the modified oligonucleotide comprises at least one modified internucleoside linkage.

Embodiment 28. The method of embodiment 27, wherein each internucleoside linkage of the modified oligonucleotide is a modified internucleoside linkage.

Embodiment 29. The method of embodiment 27 or 28, wherein at least one internucleoside linkage is a phosphorothioate internucleoside linkage.

Embodiment 30. The method of embodiment 27 or 29, wherein the modified oligonucleotide comprises at least one unmodified phosphodiester internucleoside linkage.

Embodiment 31. The method of embodiment 27, wherein each internucleoside linkage is either an unmodified phosphodiester internucleoside linkage or a phosphorothioate internucleoside linkage.

Embodiment 32. The method of any of embodiment 28, wherein each internucleoside linkage is a phosphorothioate internucleoside linakge.

Embodiment 33. The method of any of embodiments 1-32, wherein the modified oligonucleotide comprises at least one modified nucleobase.

Embodiment 34. The method of embodiment 33, wherein the modified nucleobase is a 5-methylcytosine.

Embodiment 35. The method of any of embodiments 1-34, wherein each nucleobase of each nucleoside of the modified oligonucleotide is either an unmodified nucleobase or is a 5-methylcytosine.

Embodiment 36. The method of any of embodiments 1-35 wherein the oligomeric compound comprises a conjugate group.

Embodiment 37. The method of any of embodiments 1-16 or 18-36, wherein the oligomeric compound is paired with a second oligomeric compound to form a duplex.

### I. Certain Oligonucleotides

In certain embodiments, the provided herein are oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modifed sugar moiety and a modified nucleobase.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy.

In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]n-O—, —C(Rₐ)═C(R)—, —C(Rₐ)=N—, —C(═NRₐ)—, —C(═O)—, —C(═S)—, —O—, —S(Rₐ)₂—, —S(═O)ₓ—, and —N(Rₐ)—;
wherein:
x is 0, 1, or2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J2, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

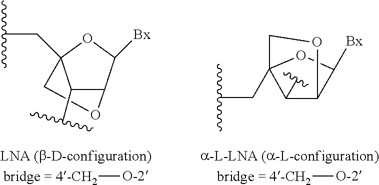

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA.

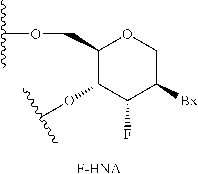

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

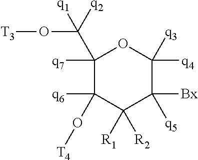

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is 0, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

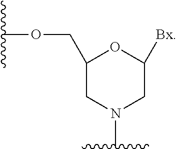

In certain embodiments, morpholinos may be modified, for example by adding or altering various substituent groups from the above morpholino structure. Such sugar surrogates are refered to herein as “modifed morpholinos.” In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

### 2. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P=O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### B. Certain Motifs

In certain embodiments, modified oligonucleotides comprise one or more modified nucleoside comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif In certain embodiments, a fully modified oligonucleotide is a uniformly modified oligonucleotide. In certain embodiments, each nucleoside of a uniformly modified comprises the same 2′-modification.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified.

In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

### C. Certain Lengths

It is possible to increase or decrease the length of an oligonuclotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target mRNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to28, 13 to29, 13 to30, 14 to 15, 14 to 16, 14 to 17, 14 to 18, 14 to 19, 14 to20, 14 to21, 14 to22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to29, 16 to30, 17 to 18, 17 to 19, 17 to20, 17 to21, 17 to22, 17 to23, 17 to24, 17 to25, 17 to26, 17 to27, 17 to28, 17 to29, 17 to30, 18 to 19, 18 to20, 18 to21, 18 to22, 18 to23, 18 to24, 18 to25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### D. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 90%, at least 95%, or 100% complementary to the second oligonucleotide or nucleic acid, such as a target nucleic acid.

### II. Certain Oligomeric Compounds

In certain embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N. Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### 1. Coniugage Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugage Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodimements, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### III. Duplexed Oligomeric Compounds

In certain embodiments, oligomeric compounds described herein comprise an oliognucleotide, having a nucleobase sequence complementary to that of a target nucleic acid. Oligomeric compounds are single-strands and in certain embodiments, oligomeric compounds are single stranded. In certain embodiments, a single-stranded oligomeric compound comprises or consists of a modified oligonucleotide and optionally a conjugate group. In certain embodiments, an oligomeric compound is paired with a second oligomeric compound to form a duplex. Such duplexed oligomeric compounds comprise a first oligomeric compound having a region complementary to a target nucleic acid and a second oligomeric compound having a region complementary to the first oligomeric compound. In certain embodiments, the first oligomeric compound of a duplexed oligomeric compound comprises or consists of (1) a modified or unmodified oligonucleotide and optionally a conjugate group and (2) a second modified or unmodified oligonucleotide and optionally a conjugate group. Either or both oligomeric compounds of a duplexed oligomeric compound may comprise a conjugate group. The oligonucleotides of duplexed oligomeric compounds may include non-complementary overhanging nucleosides.

### IV. Antisense Activity

In certain embodiments, oligomeric compounds are capable of hybridizing to a target nucleic acid, resulting in at least one antisense activity. In certain embodiments, oligomeric compounds selectively affect one or more target nucleic acid. Such selective oligomeric compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an oligomeric compound to a target nucleic acid results in recruitment of a protein that cleaves the target nucleic acid. For example, certain oligomeric compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are oligomeric compounds that are sufficiently “DNA-like” to elicit RNase H activity. In certain embodiments, one or more non-DNA-like nucleoside in the gap of a gapmer is tolerated.

In certain antisense activities, an oligomeric compound or a portion of an oligomeric compound is loaded into an RNA-induced silencing complex (RISC), ultimately resulting in cleavage of the target nucleic acid. For example, certain oligomeric compounds result in cleavage of the target nucleic acid by Argonaute. Oligomeric compounds that are loaded into RISC are RNAi compounds. RNAi compounds may be double-stranded (siRNA) or single-stranded (ssRNA).

In certain embodiments, hybridization of an oligomeric compound to a target nucleic acid does not result in recruitment of a protein that cleaves that target nucleic acid. In certain embodiments, hybridization of the oligomeric compound to the target nucleic acid results in alteration of splicing of the target nucleic acid. In certain embodiments, hybridization of an oligomeric compound to a target nucleic acid results in inhibition of a binding interaction between the target nucleic acid and a protein or other nucleic acid. In certain embodiments, hybridization of an oligomeric compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### V. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, oligonucleotides described herein are complementary to a target nucleic acid comprising a single-nucleotide polymorphism (SNP). In certain such embodiments, the oligonucleotide is capable of modulating expression of one allele of the SNP-containing target nucleic acid to a greater or lesser extent than it modulates another allele. In certain embodiments, an oligonucleotide hybridizes to a (SNP)-containing target nucleic acid at the single-nucleotide polymorphism site.

In certain embodiments, oligonucleotides are at least partially complementary to more than one target nucleic acid. For example, oliognucleotides described herein may mimic microRNAs, which typically bind to multiple targets.

### A. Complementarity/Mismatches to the Target Nucleic Acid

It is possible to incroduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligomeric compounds comprise oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligomeric compound comprising an oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. Ataxin-2

In certain embodiments, oligomeric compounds comprise or consist of any oligonucleotide comprising a region that is complementary to a target nucleic acid, wherein the target nucleic acid is Ataxin-2. In certain embodiments, Ataxin-2 nucleic acid has the sequence set forth in GENBANK Accession No. NM_002973.3 (incorporated herein as SEQ ID NO: 3), the complement of GENBANK Accession No. NT_009775.17 truncated from nucleotides 2465000 to 2616000 (incorporated herein as SEQ ID NO: 4) and GENBANK Accession No. BX410018.2 (incorporated herein as SEQ ID NO: 5).

In certain embodiments, contacting a cell with an oligonucleotide complementary to SEQ ID NO: 3, the complement of SEQ ID NO: 4, or SEQ ID NO: 5 reduces expression of Ataxin-2 mRNA. In certain embodiments, contacting a cell with an oligomeric compound complementary to SEQ ID NO: 3, the complement of SEQ ID NO: 4, or SEQ ID NO: 5 ameliroates one or more symptoms of ALS. In certain embodiments, the symptom is loss of motor function, reduced CMAP amplitude, denervation, and loss of motor neurons.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in CNS tissue, including brain tissue, such as cortex, cerebellum, and pons.

### VI. Certain Pharmaceutical Compositions

In certain embodiments, described herein are pharmaceutical compositions comprising one or more oligomeric compound or a salt thereof. In certain embodiments, the pharmaceutical composition comprises a pharmaceutically acceptable diluent or carrier. In certain embodiments, a pharmaceutical composition comprises a sterile saline solution and one or more oligomeric compound. In certain embodiments, a pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS.

In certain embodiments, pharmaceutical compositions comprise one or more or oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or f such as for sugar anomers, or as (D) or (L), such as for amino acids, etc. Included in the compounds provided herein are all such possible isomers, including their racemic and optically pure forms, unless specified otherwise. Likewise, all cis- and trans-isomers and tautomeric forms are also included unless otherwise indicated. Unless otherwise indicated, compounds described herein are intended to include corresponding salt forms.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: In Vitro Inhibition of Ataxin-2

Modified oligonucleotides that are 100% complementary to a mouse Ataxin-2 transcript were tested for their effects on mouse Ataxin-2 mRNA expression in vitro. The modified oligonucleotides listed in Table 1 target mouse Ataxin-2 pre-mRNA, designated herein as SEQ ID NO: 1 (GENBANK Accession No. NT_078458.6 truncated from nucleotides 8174000 to 8280000) and/or mouse Ataxin-2 mRNA, designated herein as SEQ ID NO: 2 (GENBANK Accession No. NM_009125.2). “Start site” indicates the 5′-most nucleoside of the indicated target sequence to which each oligonucleotide is complementary. “Stop site” indicates the 3′-most nucleoside of the indicated target sequence to which each oligonucleotide is complementary. The modified oligonucleotides listed in Table 1 were tested at various doses in bEND cells and primary mouse cortical neurons. bEND cells and neurons were electroporated or incubated, respectively, with 5 different concentrations of each modified oligonucleotide, ranging from 0.25 to 20 μM. After 24 hours for the bEND cells and after 96 hours for the neurons, RNA was isolated from the bEND cells. After an additional 72 hours, RNA was isolated from the neurons. Ataxin-2 mRNA levels were measured by quantitative real-time PCR. Mouse Ataxin-2 primer/probe set RTS3642 was used to measure mRNA levels. The primer/probe sequences are: forward primer: 5′-ACCAAAGAGTAGTTAATGGAGGTGTTC-3′ (SEQ ID NO: 11); reverse primer: 5′-AGAAGGTGGGCGAGAGGAA-3′ (SEQ ID NO: 12; and probe: 5′-CTGGCCATCGCCTTGCCCA-3′ (SEQ ID NO: 13). Ataxin-2 mRNA levels were normalized to total RNA as measured using Ribogreen. Results are presented as percent the half maximal inhibitory concentration (IC₅₀) for each oligonucleotide. The oligonucleotides in Table 1 inhibited Ataxin-2 in both cell types in a dose responsive manner.

TABLE 1
IC₅₀ values of modified oligonucleotides complementary to mouse Ataxin-2

| | | | | | | b.END | | | |
|---|---|---|---|---|---|---|---|---|---|
| | | SEQ ID NO: 1 | | SEQ ID NO: 2 | | cells | neurons | Unmodified | Modified |
| | | Start | Stop | Start | Stop | IC50 | IC50 | SEQ ID | SEQ ID |
| Isis No. | Sequence | site | site | site | site | (μM) | (μM) | NO. | NO. |
| 708135 | ^{m}Cₑₛ Gₑₒ Tₑₒ Tₑₒ Tₑₒ G_{ds} ^{m}C_{ds} | 37434 | 37453 | 714 | 733 | 7.9 | 1.4 | 14 | 46 |
| | A_{ds} T_{ds} A_{ds} G_{ds} A_{ds} T_{ds} T_{ds} | | | | | | | | |
| | ^{m}C_{ds} ^{m}Cₑₒ Aₑₒ Tₑₛ ^{m}Cₑₛ Aₑ | | | | | | | | |
| 708137 | ^{m}Cₑₛ Tₑₒ Tₑₒ ^{m}Cₑₒ Aₑₒ ^{m}C_{ds} A_{ds} | n/a | n/a | 762 | 781 | 2.8 | 1.7 | 15 | 47 |
| | T_{ds} T_{ds} T_{ds} ^{m}C_{ds} G_{ds} A_{ds} T_{ds} | | | | | | | | |
| | ^{m}C_{ds} ^{m}Cₑₒ Aₑₒ Aₑₛ ^{m}Cₑₛ Aₑ | | | | | | | | |
| 708140 | Tₑₛ ^{m}Cₑₒ Aₑₒ Tₑₒ Aₑₒ T_{ds} A_{ds} | 37965 | 37984 | 790 | 809 | 6.9 | 1.2 | 16 | 48 |
| | T_{ds} G_{ds} ^{m}C_{ds} ^{m}C_{ds} T_{ds} ^{m}C_{ds} | | | | | | | | |
| | ^{m}C_{ds} G_{ds} Tₑₒ Tₑₒ Tₑₛ Tₑₛ Tₑ | | | | | | | | |
| 708188 | Gₑₛ Tₑₒ Gₑₒ Gₑₒ Aₑₒ ^{m}C_{ds} A_{ds} | 67431 | 67450 | 1156 | 1175 | 8.4 | 6.6 | 17 | 49 |
| | ^{m}C_{ds} ^{m}C_{ds} A_{ds} ^{m}C_{ds} A_{ds} ^{m}C_{ds} | | | | | | | | |
| | ^{m}C_{ds} A_{ds} Tₑₒ Aₑₒ Aₑₛ Tₑₛ Tₑ | | | | | | | | |
| 708205 | ^{m}Cₑₛ Tₑₒ Gₑₒ Gₑₒ Aₑₒ T_{ds} T_{ds} | 67988 | 68007 | 1267 | 1286 | 7 | 3.9 | 18 | 50 |
| | ^{m}C_{ds} A_{ds} A_{ds} T_{ds} T_{ds} T_{ds} ^{m}C_{ds} | | | | | | | | |
| | T_{ds} Tₑₒ ^{m}Cₑₒ Tₑₛ Gₑₛ ^{m}Cₑ | | | | | | | | |
| 708206 | Tₑₛ Gₑₒ Tₑₒ Aₑₒ ^{m}Cₑₒ T_{ds} G_{ds} | 67999 | 68018 | 1278 | 1297 | 3.3 | 1.5 | 19 | 51 |
| | A_{ds} G_{ds} ^{m}C_{ds} A_{ds} ^{m}C_{ds} T_{ds} G_{ds} | | | | | | | | |
| | G_{ds} Aₑₒ Tₑₒ Tₑₛ ^{m}Cₑₛ Aₑ | | | | | | | | |
| 708212 | Gₑₛ Tₑₒ ^{m}Cₑₒ Aₑₒ Tₑₒ ^{m}C_{ds} A_{ds} | 68028 | 68047 | 1307 | 1326 | 3.4 | 1.3 | 20 | 52 |
| | T_{ds} T_{ds} ^{m}C_{ds} T_{ds} ^{m}C_{ds} A_{ds} A_{ds} | | | | | | | | |
| | G_{ds} Gₑₒ Gₑₒ ^{m}Cₑₛ Gₑₛ Aₑ | | | | | | | | |
| 708213 | Gₑₛ Gₑₒ Tₑₒ ^{m}Cₑₒ Aₑₒ T_{ds} ^{m}C_{ds} | 68029 | 68048 | 130 | 1327 | 10.3 | 0.8 | 21 | 53 |
| | A_{ds} T_{ds} T_{ds} ^{m}C_{ds} T_{ds} ^{m}C_{ds} A_{ds} | | | | | | | | |
| | A_{ds} Gₑₒ Gₑₒ Gₑₛ ^{m}Cₑₛ Gₑ | | | | | | | | |
| 708217 | Tₑₛ Tₑₒ ^{m}Cₑₒ Tₑₒ Tₑₒ ^{m}C_{ds} ^{m}C_{ds} | 68043 | 68062 | 1322 | 1341 | 4.4 | 7.5 | 22 | 54 |
| | T_{ds} ^{m}C_{ds} A_{ds} ^{m}C_{ds} T_{ds} ^{m}C_{ds} | | | | | | | | |
| | ^{m}C_{ds} G_{ds} Gₑₒ Tₑₒ ^{m}Cₑₛ Aₑₛ Tₑ | | | | | | | | |
| 708299 | Gₑₛ Gₑₒ Tₑₒ Gₑₒ Aₑₒ T_{ds} G_{ds} T_{ds} | 75837 | 75856 | 2233 | 2252 | 4.2 | 3.4 | 23 | 55 |
| | T_{ds} T_{ds} ^{m}C_{ds} A_{ds} ^{m}C_{ds} T_{ds} T_{ds} | | | | | | | | |
| | Gₑₒ ^{m}Cₑₒ Tₑₛ Tₑₛ Tₑ | | | | | | | | |
| 708307 | Gₑₛ Tₑₒ ^{m}Cₑₒ Aₑₒ Tₑₒ ^{m}C_{ds} A_{ds} | 76374 | 76393 | 2303 | 2322 | 3.6 | 3.4 | 24 | 56 |
| | A_{ds} T_{ds} ^{m}C_{ds} T_{ds} G_{ds} T_{ds} T_{ds} | | | | | | | | |
| | T_{ds} Tₑₒ ^{m}Cₑₒ Tₑₛ Gₑₛ Tₑ | | | | | | | | |
| 708318 | Gₑₛ Tₑₒ Gₑₒ Aₑₒ ^{m}Cₑₒ T_{ds} T_{ds} | 85004 | 85023 | 2400 | 2419 | 3.5 | 1.3 | 25 | 57 |
| | T_{ds} T_{ds} ^{m}C_{ds} T_{ds} ^{m}C_{ds} ^{m}C_{ds} T_{ds} | | | | | | | | |
| | T_{ds} ^{m}Cₑₒ Tₑₒ ^{m}Cₑₛ Tₑₛ Aₑ | | | | | | | | |
| 708320 | Gₑₛ ^{m}Cₑₒ Tₑₒ Tₑₒ ^{m}Cₑₒ ^{m}C_{ds} G_{ds} | 85032 | 85051 | 2428 | 2447 | 6.3 | 3.5 | 26 | 58 |
| | T_{ds} T_{ds} T_{ds} T_{ds} A_{ds} T_{ds} ^{m}C_{ds} T_{ds} | | | | | | | | |
| | Tₑₒ Tₑₒ Aₑₛ Aₑₛ Tₑ | | | | | | | | |
| 708321 | Aₑₛ Gₑₒ ^{m}Cₑₒ Aₑₒ ^{m}Cₑₒ T_{ds} T_{ds} | 85039 | 85058 | 2435 | 2454 | 9.6 | 5.7 | 27 | 59 |
| | G_{ds} ^{m}C_{ds} T_{ds} T_{ds} ^{m}C_{ds} ^{m}C_{ds} G_{ds} | | | | | | | | |
| | T_{ds} Tₑₒ Tₑₒ Tₑₛ Aₑₛ Tₑ | | | | | | | | |
| 708325 | Tₑₛ Gₑₒ ^{m}Cₑₒ Tₑₒ Gₑₒ ^{m}C_{ds} T_{ds} | 85067 | 85086 | 2463 | 2482 | 7.3 | 1.7 | 28 | 60 |
| | G_{ds} ^{m}C_{ds} T_{ds} G_{ds} T_{ds} ^{m}C_{ds} A_{ds} | | | | | | | | |
| | A_{ds} Tₑₒ Gₑₒ Aₑₛ Aₑₛ Aₑ | | | | | | | | |
| 708343 | Aₑₛ Aₑₒ Tₑₒ Gₑₒ Tₑₒ ^{m}C_{ds} G_{ds} | 86390 | 86409 | 2662 | 2681 | 3.7 | 2.9 | 29 | 61 |
| | A_{ds} T_{ds} T_{ds} T_{ds} ^{m}C_{ds} ^{m}C_{ds} T_{ds} | | | | | | | | |
| | A_{ds} Aₑₒ ^{m}Cₑₒ ^{m}Cₑₛ Tₑₛ Gₑ | | | | | | | | |
| 708378 | Gₑₛ Aₑₒ ^{m}Cₑₒ ^{m}Cₑₒ Aₑₒ A_{ds} A_{ds} | 92235 | 92254 | 3083 | 3102 | 6.2 | 2.8 | 30 | 62 |
| | G_{ds} G_{ds} ^{m}C_{ds} T_{ds} G_{ds} A_{ds} T_{ds} | | | | | | | | |
| | T_{ds} Gₑₒ Gₑₒ Gₑₛ Aₑₛ Aₑ | | | | | | | | |
| 708379 | Gₑₛ Gₑₒ Aₑₒ ^{m}Cₑₒ ^{m}Cₑₒ A_{ds} A_{ds} | 92236 | 92255 | 3084 | 3103 | 5.5 | 2.3 | 31 | 63 |
| | A_{ds} G_{ds} G_{ds} ^{m}C_{ds} T_{ds} G_{ds} A_{ds} | | | | | | | | |
| | T_{ds} Tₑₒ Gₑₒ Gₑₛ Gₑₛ Aₑ | | | | | | | | |
| 708386 | ^{m}Cₑₛ Aₑₒ Gₑₒ Gₑₒ Aₑₒ ^{m}C_{ds} T_{ds} | 92966 | 92985 | 3132 | 3151 | 4.7 | 1.1 | 32 | 64 |
| | G_{ds} T_{ds} A_{ds} ^{m}C_{ds} A_{ds} ^{m}C_{ds} A_{ds} | | | | | | | | |
| | T_{ds} Gₑₒ Aₑₒ Gₑₛ Gₑₛ Aₑ | | | | | | | | |
| 708393 | ^{m}Cₑₛ Aₑₒ ^{m}Cₑₒ Tₑₒ Aₑₒ A_{ds} A_{ds} | 93025 | 93044 | 3191 | 3210 | 7.2 | 2.9 | 33 | 65 |
| | ^{m}C_{ds} ^{m}C_{ds} A_{ds} G_{ds} G_{ds} ^{m}C_{ds} T_{ds} | | | | | | | | |
| | G_{ds} Aₑₒ Gₑₒ ^{m}Cₑₛ Aₑₛ Tₑ | | | | | | | | |
| 708458 | ^{m}Cₑₛ Tₑₒ Gₑₒ Tₑₒ Tₑₒ A_{ds} G_{ds} | 104505 | 104524 | 4011 | 4030 | 5.5 | 3.9 | 34 | 66 |
| | ^{m}C_{ds} A_{ds} T_{ds} T_{ds} ^{m}C_{ds} ^{m}C_{ds} T_{ds} | | | | | | | | |
| | A_{ds} Tₑₒ ^{m}Cₑₒ Aₑₛ Gₑₛ Aₑ | | | | | | | | |
| 708461 | Gₑₛ Tₑₒ ^{m}Cₑₒ ^{m}Cₑₒ Aₑₒ A_{ds} A_{ds} | 104533 | 104552 | 4039 | 4058 | 5.2 | 2.1 | 35 | 67 |
| | A_{ds} ^{m}C_{ds} A_{ds} T_{ds} ^{m}C_{ds} ^{m}C_{ds} T_{ds} | | | | | | | | |
| | ^{m}C_{ds} ^{m}Cₑₒ Aₑₒ ^{m}Cₑₛ Tₑₛ Gₑ | | | | | | | | |
| 708462 | Tₑₛ Gₑₒ ^{m}Cₑₒ ^{m}Cₑₒ Tₑₒ ^{m}C_{ds} T_{ds} | 104545 | 104564 | 4051 | 4070 | 3.1 | 0.5 | 36 | 68 |
| | A_{ds} ^{m}C_{ds} T_{ds} ^{m}C_{ds} G_{ds} G_{ds} T_{ds} | | | | | | | | |
| | ^{m}C_{ds} ^{m}Cₑₒ Aₑₒ Aₑₛ Aₑₛ Aₑ | | | | | | | | |
| 708463 | ^{m}Cₑₛ ^{m}Cₑₒ ^{m}Cₑₒ Tₑₒ Aₑₒ ^{m}C_{ds} | 104552 | 104571 | 4058 | 4077 | 7.3 | 4.5 | 37 | 69 |
| | A_{ds} T_{ds} G_{ds} ^{m}C_{ds} ^{m}C_{ds} T_{ds} ^{m}C_{ds} | | | | | | | | |
| | T_{ds} A_{ds} ^{m}Cₑₒ Tₑₒ ^{m}Cₑₛ Gₑₛ Gₑ | | | | | | | | |
| 708468 | Aₑₛ Gₑₒ ^{m}Cₑₒ ^{m}Cₑₒ Aₑₒ ^{m}C_{ds} | 104562 | 104581 | 4068 | 4087 | 1.7 | 2.6 | 38 | 70 |
| | A_{ds} A_{ds} G_{ds} T_{ds} ^{m}C_{ds} ^{m}C_{ds} ^{m}C_{ds} | | | | | | | | |
| | T_{ds} A_{ds} ^{m}Cₑₒ Aₑₒ Tₑₛ Gₑₛ ^{m}Cₑ | | | | | | | | |
| 708481 | Gₑₛ Tₑₒ Tₑₒ ^{m}Cₑₒ Aₑₒ T_{ds} G_{ds} | 104665 | 104684 | 4171 | 4190 | 6.2 | 1.3 | 39 | 71 |
| | A_{ds} ^{m}C_{ds} T_{ds} ^{m}C_{ds} T_{ds} ^{m}C_{ds} A_{ds} | | | | | | | | |
| | A_{ds} Gₑₒ Gₑₒ Gₑₛ ^{m}Cₑₛ ^{m}Cₑ | | | | | | | | |
| 708491 | Aₑₛ ^{m}Cₑₒ Tₑₒ Tₑₒ ^{m}Cₑₒ T_{ds} G_{ds} | 104771 | 104790 | 4277 | 4296 | 12.8 | 7 | 40 | 72 |
| | T_{ds} ^{m}C_{ds} A_{ds} A_{ds} G_{ds} T_{ds} T_{ds} | | | | | | | | |
| | T_{ds} Aₑₒ Gₑₒ Tₑₛ Aₑₛ Aₑ | | | | | | | | |
| 708503 | Aₑₛ Gₑₒ Tₑₒ Tₑₒ Tₑₒ T_{ds} ^{m}C_{ds} | 104875 | 104894 | 4381 | 4400 | 7.7 | 2.2 | 41 | 73 |
| | ^{m}C_{ds} ^{m}C_{ds} T_{ds} T_{ds} A_{ds} A_{ds} ^{m}C_{ds} | | | | | | | | |
| | T_{ds} Tₑₒ Aₑₒ Aₑₛ Aₑₛ Aₑ | | | | | | | | |
| 708511 | Gₑₛ Aₑₒ ^{m}Cₑₒ Aₑₒ Aₑₒ A_{ds} A_{ds} | 8800 | 8819 | n/a | n/a | 5.8 | 5.1 | 42 | 74 |
| | T_{ds} G_{ds} A_{ds} G_{ds} ^{m}C_{ds} ^{m}C_{ds} A_{ds} | | | | | | | | |
| | G_{ds} Tₑₒ Tₑₒ Tₑₛ Tₑₛ Aₑ | | | | | | | | |
| 708512 | Aₑₛ Aₑₒ Tₑₒ ^{m}Cₑₒ Tₑₒ G_{ds} T_{ds} | 11550 | 11569 | n/a | n/a | 6.3 | 2.8 | 43 | 75 |
| | G_{ds} T_{ds} T_{ds} T_{ds} T_{ds} ^{m}C_{ds} ^{m}C_{ds} | | | | | | | | |
| | ^{m}C_{ds} Tₑₒ Tₑₒ Tₑₛ ^{m}Cₑₛ Gₑ | | | | | | | | |
| 708522 | Tₑₛ Aₑₒ Gₑₒ ^{m}Cₑₒ Aₑₒ ^{m}C_{ds} ^{m}C_{ds} | 39657 | 39676 | n/a | n/a | 4.4 | 1.4 | 44 | 76 |
| | A_{ds} G_{ds} G_{ds} T_{ds} A_{ds} T_{ds} G_{ds} | | | | | | | | |
| | ^{m}C_{ds} ^{m}Cₑₒ ^{m}Cₑₒ Aₑₛ Tₑₛ Gₑ | | | | | | | | |
| 708540 | ^{m}Cₑₛ Aₑₒ Tₑₒ Gₑₒ Gₑₒ T_{ds} T_{ds} | 89861 | 89880 | n/a | n/a | 5.3 | 3.8 | 45 | 77 |
| | A_{ds} T_{ds} ^{m}C_{ds} T_{ds} A_{ds} T_{ds} G_{ds} | | | | | | | | |
| | G_{ds} ^{m}Cₑₒ Aₑₒ Gₑₛ ^{m}Cₑₛ Aₑ | | | | | | | | |

Subscripts: “e” indicates a 2′-MOE modified nucleoside, “d” a 2′-deoxynucleoside, “o” indicates a phosphate internucleoside linkage, and “s” indicates a phosphorothioate internucleoside linkage. Superscript “m” preceding a “C” indicates a 5-methylcytosine.

### Example 2: In Vivo Reduction of Ataxin-2 mRNA in Mice

Select oligonucleotides described in Table 1 above were tested for antisense activity in vivo. Female C571B1/6 mice each received a single intracerebroventricular bolus (ICVB) injection of 500 μg of one of the oligonucleotides listed in the table below or PBS vehicle alone. Each treatment group consisted of 3 or 4 animals. Two weeks later, the animals were sacrificed, and RNA was isolated from the cortex, cerebellum, and spinal cord. Ataxin-2 mRNA levels were measured by RT-qPCR, as described in Example 1, and the resulting values were normalized to GAPDH levels. The average inhibition of Ataxin-2 mRNA levels for each treatment group, relative to the PBS treated group, are shown in the table below. The results show that the oligonucleotides in the table below inhibited Ataxin-2 mRNA levels in the cortex, cerebellum, and the spinal cord.

TABLE 2
Inhibition of Ataxin-2 in wild type mice

| | Inhibition of Ataxin-2 mRNA (%) | | |
|---|---|---|---|
| Isis No. | Cortex | Cerebellum | Spinal Cord |
| 708137 | 63 | 60 | 76 |
| 708140 | 57 | 57 | 70 |
| 708206 | 45 | 50 | 77 |
| 708212 | 31 | 27 | 56 |
| 708213 | 49 | 33 | 69 |
| 708318 | 91 | 75 | 89 |
| 708386 | 39 | 29 | 62 |
| 708462 | 78 | 66 | 81 |
| 708468 | 52 | 27 | 64 |
| 708481 | 71 | 67 | 82 |
| 708522 | 50 | 48 | 62 |

### Example 3: Effect of Ataxin-2 Modified Oligonucleotides on CMAP in an ALS Model, SOD1^{G93a} Mice

Isis No. 708137 (see Table 1 above) was tested for its effects on compound muscle action potential (CMAP) in SOD1^{G93A}mutant mice. SOD1^{G93A} mutant mice express the human G93A SOD1 variant and early denervation (or loss of motor units) was reported in the fast-twitch muscles of the SOD1^{G93A} mutant mice (Frey et al., 2000; Pun et al., 2006). In order to test the effect of Isis No. 708137 on the motor units of the fast-twitch muscle tibialis anterior (TA), CMAP was measured, which is a functional measurement of motor units represented by the summation of all action potentials in a given area of the muscle upon stimulation of the sciatic nerve. Baseline CMAP measurements were performed on both the left and right TA muscles on five week old wild type and SOD1^{G93A} mutant mice one day before each mouse received an ICVB injection of 500 μg of Isis No. 708137 or of vehicle alone. Each treatment group consisted of 11 or 12 animals. CMAP measurements were also performed at 7, 9, and 10 weeks of age (2, 4, and 5 weeks following oligonucleotide treatment). Each CMAP measurement was performed by stimulating the sciatic nerve percutaneously by single pulses of 0.1 ms duration (VikingQuest NCS/EMG Portable EMG machine) delivered through a pair of needle electrodes placed at the sciatic notch. The CMAP was recorded with the recording electrode placed subdermally on the muscle belly of the TA muscle. A reference electrode was placed near the ankle and ground electrode at the midline. Disposable monopolar needle electrodes (25 mm, 28G; catalog #902-DMF25-TP, Natus Medical Inc., San Carlos, CA) were used for both stimulating and recording. The CMAP trace used for analysis from a given animal or leg was obtained from 4 stimuli. The CMAP value of an individual animal at a given time point represents the averaged peak-to-peak amplitude of both left and right legs. The results shown in the table below are the average CMAP values of all animals in each treatment group. Data were analyzed with two-way ANOVA. The results in the table below show that treatment with an oligonucleotide targeting Ataxin-2 partially rescued motor function in SOD1^{G93A} mutant mice. The differences between the treated and untreated SOD1^{G93A} mutant mice were determined to be significant at 7, 9, and 10 weeks of age.

TABLE 3
CMAP in SOD1^{G93A} mice

| | | Age | |
|---|---|---|---|
| Mice | Treatment | (weeks) | CMAP (mV) |
| WT | Vehicle | 5 | 84 |
| | | 7 | 89 |
| | | 9 | 86 |
| | | 10 | 84 |
| SOD1^{G93A} | Vehicle | 5 | 83 |
| | | 7 | 73 |
| | | 9 | 49 |
| | | 10 | 43 |
| | Isis No. | 5 | 84 |
| | 708137 | 7 | 83 |
| | | 9 | 65 |
| | | 10 | 52 |

### Example 4. Effect of Ataxin-2 Modified Oligonucleotides on Denervation in an ALS Model, SOD^{193A}Mice

Histological analysis of neuromuscular junctions was performed on anterior tibialis muscles of SOD1^{G93A} mice treated with Isis No. 708137 or vehicle at 9 weeks of age as described in Example 3 above.

Anterior tibialis muscles were dropped-fixed in 10% neutral formalin at room temperate for 1 hour and subsequently transferred to PBS at 4° C. overnight to remove fixatives. Tissues were then cryoprotected by immersing in 30% sucrose in PBS at 4° C. overnight prior to embedding in Tissue Tek OCT compound for cryosectioning. Twenty-μm thick sections were stained for neuromuscular junctions (NMJs) with rabbit anti-vesicular acetylcholine transporter (VAChT) antibody (1:200; synaptic system) followed by Alexa Fluor 594-conjugated α-bungarotoxin (Invitrogen) for acetylcholine receptors (AChRs). NMJs were visualized and imaged with confocal microscope (Olympus). Fully innervated NMJs were defined by complete overlap of presynaptic (i.e., VAChT) and postsynaptic endplate (i.e., AChR) labeling. Partially innervated NMJs were the postsynaptic endplates with partial occupancy of presynaptic terminals. Denervated NMJs were endplates devoid of presynaptic labeling. For each sample, 50-100 NMJs were examined and the proportions of each NMJ category were quantified. Data was the average of 4 samples from each treatment group. The results in the table below show that treatment with an oligonucleotide complementary to Ataxin-2 partially ameliorated denervation in SOD1^{G93A} mutant mice.

TABLE 4
Innervation in SOD1^{G93A} mice

| | | |
|---|---|---|
| Mice | Treatment | Innveration (% of NMJs) |

| | | | |
|---|---|---|---|
| SOD1^{G93A} | Vehicle | Fully Innervated | 32 |
| | | Partially Innvervated | 11 |
| | | Denervated | 57 |
| SOD1^{G93A} | Isis No. 708137 | Fully Innverated | 44 |
| | | Partially Innverated | 24 |
| | | Denervated | 32 |

1.-66. (canceled)

67. A method comprising administering to an animal having a loss of motor function an oligomeric compound comprising a modified oligonucleotide consisting of 12 to 30 linked nucleosides, wherein the nucleobase sequence of the modified oligonucleotide is at least 90% complementary to the nucleobase sequence of an Ataxin-2 nucleic acid, wherein the loss of motor function in the animal is associated with a SOD1 or TDP43 mutation, and wherein the animal does not have a polyglutamine (polyQ) expansion in the Ataxin-2 protein.

68. The method of claim 67, wherein the nucleobase sequence of the modified oligonucleotide is at least 90% complementary to an Ataxin-2 nucleic acid having the nucleobase sequence of SEQ ID NO: 3, SEQ ID NO: 4, or SEQ ID NO: 5.

69. The method of claim 67, wherein administration of the oligomeric compound slows progression of the loss of motor function.

70. The method of claim 67, wherein administration of the oligomeric compound improves motor function.

71. The method of claim 67, wherein the amount of Ataxin-2 mRNA and/or Ataxin-2 protein is reduced in the animal following administration of the oligomeric compound.

72. The method of claim 67, wherein the modified oligonucleotide comprises at least one modified nucleoside comprising a bicyclic sugar moiety, at least one modified nucleoside comprising a non-bicyclic sugar moiety, or at least one modified nucleoside comprising a sugar surrogate.

73. The method of claim 72, wherein the bicyclic sugar moiety has a 2′-4′ bridge, wherein the 2-4′ bridge is selected from —O—CH₂—; —O—CH₂—CH₂—; and —O—CH(CH₃)—.

74. The method of claim 72, wherein the non-bicyclic sugar moiety comprises a 2′-MOE or 2′-OMe.

75. The method of claim 72, wherein the sugar surrogate is selected from a morpholino, a PNA, a F-HNA, a THP, or a modified THP.

76. The method of claim 67, wherein the modified oligonucleotide has a sugar motif comprising:
a 5′-region consisting of 1-5 linked 5′-region nucleosides;
a central region consisting of 6-10 linked central region nucleosides; and
a 3′-region consisting of 1-5 linked 3′-region nucleosides; wherein each of the 5′-region nucleosides and each of the 3′-region nucleosides comprises a modified sugar moiety and each of the central region nucleosides comprises an unmodified DNA sugar moiety.

77. The method of claim 67, wherein at least one internucleoside linkage is a phosphorothioate internucleoside linkage.

78. The method of claim 67, wherein each internucleoside linkage is either an unmodified phosphodiester internucleoside linkage or a phosphorothioate internucleoside linkage.

79. The method of claim 67, wherein the modified oligonucleotide comprises at least one 5-methylcytosine.

80. The method of claim 67, wherein the oligomeric compound comprises a conjugate group.

81. The method of claim 67, wherein the oligomeric compound is paired with a second oligomeric compound to form a duplex.

82. The method of claim 67, wherein the administering is to the central nervous system.

83. The method of claim 82, wherein the administering is intrathecal administration or intracerebroventricular administration.

84. The method of claim 67, wherein the animal has ALS associated with a SOD1 or TDP43 mutation.

85. The method of claim 67, wherein the nucleobase sequence of the modified oligonucleotide is at least 95% complementary to an Ataxin-2 nucleic acid having the nucleobase sequence of SEQ ID NO: 3, SEQ ID NO: 4, or SEQ ID NO: 5.

86. The method of claim 67, wherein the nucleobase sequence of the modified oligonucleotide is 100% complementary to an Ataxin-2 nucleic acid having the nucleobase sequence of SEQ ID NO: 3, SEQ ID NO: 4, or SEQ ID NO: 5.

87. The method of claim 67, wherein the animal does not have a CAG expansion in the ATXN2 gene of more than 22 repeats.

88. The method of claim 67, wherein the animal is a human.


SEQUENCE LISTING (77 sequences)

SEQ ID NO: 1 - DNA, 106001 bp, Mus musculus
  source 1..106001: mol_type = genomic DNA
taattattcacaggtggaccaggggtgtggagctggagaggcaatgctatactccctatctaaaactgacaccccagatccatttttctctcttttttttttaaagtcatatttatggccagtcatggtgaagcaggtctttgatctcagcaggcaggtggatctatgtgagttccagaacagccagggctgttacacagagaaaccctgtctcggaaaaaaccaaacaagaacaaaaaccaccaccgacaaggtcatacttatattgcttatatatgtgtggcacaatccaagggtggagggctggggacaattccaagcatttaagttttcttcttccacatgccagctccagggttgagagttaggtggtgagcttaggcagcctgggcctttaccttctgagctatctgctagctacttaaaaaaaaaaaaatccatgtagaccaacaagtctggaactcaagagatccagttgcctgtccagagctgggattaaaggcatgtgccataaccaggggtaactcttcattgaattgccccttcttgagactgggtctcttgtagcttagcctggcctagaattccctcatgaaggtgaagatgaccttgactgtctaatcctccttccatttcccaagtgctcgtactacaggcaagaacaccatgcctgcctttaattcactggaatgactttaataggttgagggtttttttttttccctcggcccatcaccttcttaagcaatttcacttagaaaagaaagaaaaaaaaaaaaaccaatttaaactcatttgaaaaaagttaaccatttgtttccaatttaattacaacaccttaaaaaaaaaaacggaccagtgtttggtggccttcccgagttaaagtaaaagtttttaaaaggggtcaaggtctaaagttgacattcttgggaaagtctgtttagtcttcaccgagggagaccccatcacccagttctactaccaatcaggtccatttcccttcgggtcccctgaaggaggaactaagaagtccgagcgagctagggggaaaaaaaaaaagagggttcaaatgaaggtcagagacccaacagttcgccggggtgaataaagtggggcaaagtgaagcagctggcggtggcttctgaggcggcaagcttccccacgctggcagggcaagtcggcacgcagtggcacctgggggcctactgtgtgccaggccccgattgcaggtccccgccccgaaggtgcccgcaggccatcctctaagttgtgatttggtggggtgggggtggggtgaaatcaaggtcctccaagggggtcgaggacacccgagcagctcacggcgcccgtccccgaagcagatcccgaagttcagacccgccttggcgagggcggatccctcctggccccctccacggccctagttcgaccccggggagagcgaacgcggcccagcgctgcgccccgctcctcacgtgtccctgatcggccccccgggccgtctcacgttcgacttccgcctgacccttccgacttccggtaaagagtccccccccccacccccgacacctcccccgccccgggtctgtttcgcatctccgcgccctccaggcccgcgtcaaccccgtccgcgtccgccagcccgggtcccatgcgttcgtccaccgccgccgctcagcggcccgcggcgggggaccccgagccgcgccgcccggcgggctgggccgcgcggcgctcgctcccgcggacggcgcggcgcggcgggcggggcggcgcggtggcgtatccctccgccggccctcccccgcgcggccccggcgcccctccccgcgggccgcgctcgccaccctgcgcctcagactgttttggtagcaacggccacggcgcgtcccggcccggctcccggcggctgctcggtgtctgcgggcctccccgccccttcgtcgttgtcctgctgcctctggcccccgcggccacgccggcccgcgcctgcccgcccggcgtccgcgcgtccccgccgcgctccggcgtctcctcctcggcgcgcccggcacccggctgtccccgcccggcgtgcgagccggtgtatgggccgctcaccatgtcgctgaagccgcagccgcagccgcccgcgcccgccactggccgcaagcccggcggcggcctgctctcgtcgcccggcgccgcgccggcctcggccgcggtgacctcggcttccgtggtgccggccccggccgcgccggtggcgtcttcctcggcggccgcgggcggcgggcgtcccggcctgggcaggtgggtgtcagctcccccagccccctccgcgccgggccgatcccgcgccgctcgtcccctcccccgcctcgcgccccgtgaccggccggcttcgcgatgtgccggggggcccgcggaggggcgggaggcgccggcagattccttcgggcttctcgggccgtgtccagcctcccgcaccatcgcgtcccctccccccagacggccaagatggacggccctggcggccccctgcccctcccgggtgtgccaggggcgattggtatcccgccccacccccttgctgcgcccccccccccgatagaagggcggctgagcctgggtgcgggtcgcagggggtggggggatcctctcctgaaatgagcctctttgccgggtggggagtcgggagggaggaggggagattgaggcccagccgccccaggtagggtcaagccgggtgtcctgcatatctatcccccgccccgcagaccctgggaaggtgggcgacggaggcgaggtgggtgccagcggcctaacgcacccatccccctttagatctggggcggcggggttcccgcagcgggccgttcttcccctccccccccccaactggggaaggggtgggagtgcccggctccctccaggcccgcctccctccttccccgcaaggtcccctgcgcgcgggtggtggccgatccgcattgctgttcctcgtccgcggagcagaaggcgcctttgaatagaggtgggcgagggccggactcttgaggtgggttggccgcgggctctggacacgcaccgcattttccctcccaaccccccccccccccggcccagttcctgcaatttttaggggtgaggcgccgcaacagaggagttggtgcgggtccgaaggcaacaactcccgagtgttggcttgcgccctggtccccctctccccttccctgggcccgatctcgttggtaggctggggtgctgtcctgtgtgactggactctggagggtgtgtgcgtgcgggggtggggagtggtgggggtgcttgtcagttcgtatttcacgaactaagaaaatgcttagtgttaaaacggagaggcagatgccaatagactccattccattgtggccggtgtccttaacttgggaaacgccgccgccagagcttaccaagggcatgcaagtccttttcccctgtgcccctgtgttacaggcactactgtgcctccctctggcctaggcctggctggcagggcctggagcagcctcctccttaaatagacatccaggacttaggaggtgggcaaggggatttttcttgtacggagttttcttcttagccgcattgtcctgctgtccgtttccagagagtgttcctctacagcagaagttaacccctagggtgaatcagagccgaggttttagggaattcttctgggaacagtatgttttttactttattcctccacactcccaattttccctaacatgaggcatttcaaaccctgcttagcttgcctcgttcaagttaaaccatcattttatttccccatctctgtgaagttagggttttgtaagttgtttcttttctgtccttgacttttagtgaaaaacgaaaagatacttaaatgtaactttaaggatgcatcctgtttttctttgctttttcatgttttctctttagttatcagaaagtgacgtgtatgtcactttcactcaaatacaaattaaagctaaagttcctaggcttcaccagctctctttaggcttaaacattagatggtagctgagttgactccacagggaggtgagcagggtgtgagtgccagcaatctttttctgcagttgaggtgctgcaaaagtgaaaggcatttcgagtcttccttgcttcaagttctttcacttgaagcctaggtttttttttttttaagagttttcgtccaatactggtactcagaatgtgtgagtttgttctgtacagagccttggttaatgtcattctctcattactgtctctgtgggttccttgtaaatccattataacttgtagagttttgcttttgtgtgtgtgtgatttaaaataggactggctgcactgaggaaaggaggtgtagcattggggttggtgaggtgcttcagggggtaagggcaatcaactgggattcccataccaccaaccaccaccaccaccaccaccaccaccaccacctcagcaggaagcaactgactcctgctgcaagctgtcctctgacctctacacacctgccagggcatgttcccctcctccacagtaaataaacttaaaaaagaaaaggcgtcatgtttgagatagcaaaatgttcttcccagcctagaagctcactaaagcagtctttcacttctgggcctgcccagagcctggagcctaacccagggccttgcttttgctactcaggcagcctgggtctgacttgatttttgtgtgaatggaattttgtgatttttgagaagagttttttcttgtcaacactcaagaggccactttcttagaaaagaactgtgtgtgatcactttttaagaatcttacgggctggcgagatggctcagtggttaagagcgccgactgctctttcgacagtcctgagttcaaatcccagcaaccacatggtggttcacaaccatctgtaacgaaatctgatgccctcttctggagtatctgaggacagctacagtgtacttacatgtaataaataaatcttaaaaaaaaaaaaaagaatgttacacagttgcactggagattctacttcttactcttaactttgaccttttgtgaggctgtgctcttgtgggatcgaggcttcccctttaatcgggagggtttttttttttttttttttttttaaacagtagtgtacctgctaaactaagtttctccctgccggctcggactttggtcttgacgttcctggttttgtggaggtggtatggtgaactttttcatactcggagctttttgtttcatcttaatttttccctagagaaagtgcccaggagtggtttcactaggtcaaggactcttaaggttttatggctctcacaagctgtgccatgctatttctcagaagactcagtgggttctggagccaagaaggcagctctttgggggatgggctctgtggacttgttggatgctcttggggatgttgcattcaacaaggcatgtgctttcatggatacgtttcctggtcgtttctacttttaaagatttatttttgtatgtataagtgctttgcccgtttgtatggtatgtgtgcccagggtccacagagaccagaagaagagggccctttattccctagaactgcagtgacagactgcagtgaacaccatgtggtcctctgcaagagcagccagcctttctaaccctgagccattcatcctgtccctcttgattatgtctctttgaagttttccaggattgttttttatgtgtatggatgttttgtgtgcatgtttgtaagtgtcttcatactttactgatgcccacagagaccaggagtgggctctggatcccctgggatggagttagggatagttgtgaggccaccatgtaggtgctaggttcctctgcaagagcagccatgccgggcttggtgccacatgcctttcatcccagcccaagagatggggcaagtgaatctttggaacttgaggccagcctggtctatatagggagtagacttacaacaagtgctcttaacctcttcagcccttgtatttgaattttacatgagcttgcactgagactgtgtatgaagatgtttgggggtctgtagtgtataatgccagtgcagatccatggtttctgaaggaactgttctaaaactttctggctgttaaaaaattgttaatccttttttatttgtgttattagctgttagtttatgatcacttttgaaaaaaaaagttagcttgtggtgaagcatggtggtaggattaactgaaggaggttgaggcaggaggatcatgagttacaggccagcctgggctacacatgtactgttcagaggtggtgtacacctttgatcctaacaccggggaggcagacaggcagtctgctgctgtaccgagcagccagggctacgcagagaaaccctgtcttggaaaaacaagtttaatttgtttatgcatacctcttgtctgtctggtgcctgtggaggtcagaagaggaggccagatgccctagagctgtagttatggacggtcattgggccactgtgtggatgctgggaactgagcccaggtcctctgcaagagcggccagtgctcttaactgctgagctacctgtccacaaccatgcctttattttttgagttcagatcttttgtatccgattgttcttgtccttgaaactttggtgtgcctgcccaaccccctcccaactgttgggattgtaagtgtgcacctgcctactttatgcagtgttggggtggtcattcagagcttgtctgcatgctaggcacacgttgtagctacatcctcagctcctagttagtgactgcactgggagtcatcatgggttaattcatatccctgccttcctttcctctttattgggtttagtggttcgaggggcagcatctactttgtagaccaggctgctcaaaattttgtttgctttctttgttttgagacaaatttgtagccttgggcttaaaggcttgtatctgtcactcctggcttataggcttagacatttttttaaatttactttttaaaatttatgtgtgtatatgtgagcttgtgtggttgtgtaccacatgtgtgcaggagccctgaggaaaaccagaccagggcactgagttggagatggaggcagttgtgagctgccatgtgggtactgtggactgaacctgtgtcgtcttctgggataggtaagcatttgtaaggaaggagtcagctctcaagtgtgacttgtagcttttgtttttgtttttgtattctttttcagacagggtctcttacacaattgtgacaatcctgaaagtcactgtgtagtcccggctggtctcctaattcagagagatctacctgtctctgccttctcaggtgcactctcaagcctgggttagttttggatttttagttgttgttgttgtttttgacagggtttctttgtgtaatagccctgctgtcctggaacttgctttgcagtctaggctgatctctaagagatagacctgagtgctgggattagaggcatgggccaccattgcctggcttgcctggaactccttgaagcatgcatcttcttgcccctgcctcaggcaccatggcaggctcccacacttactttaaaaaaaaaaaatgtagcctaggctggctcacaagcacatttgggatttcaggtgtgtaccaccatcctctacacttcacactttaaacaccttttttttttctttaaaattacatttgcagggcctggagaaatggtttagtggttaagagcactggccactcttccagtggactggggttaagctccaagcacctacaagacagctcgcaactgtctgtaactcctgttccagggatctgactcccttacacatgcaggcaagacaccagcatacataaaatacaaaataaattacattttgagagagagagagagaatacatgaatgtgtgtgtgtgtgttggtaaatttcacagttgtgtgattgaagatcccaggacaacttggaggagctggttgtgggccagcgagatggaatccaggttacctgccagatgccagaccgggggcaaatgcctttctctttaagccttcttaccaaccctctcactttttaagaactttctctgctttaatcccagcatttaggaggcagaggcaggtggatctctgagttcacggccagcctggtctacagagttccaggacagccagggctacacagagaaacctcatcttggaaaaacaaaacaaaaaaccccccaccaaaacacaaagaatgttctctgatttctcagttttgttttttcagtttttttatggctttaatctctaaagtcattgcatcttagaaattgttcatctcacttccattaaatgtcagagatatctccataagttcatatccctcaagtgcttacagcaccacacagcatacattgttgagtatctgtctgtaaggcagtgttgagttctttccatgtactaatgactaaaccaaaaatatttcctctcctggagtttacattcctcttttgagccttgaagttagcgccatgtgtggccccgactgtcctgtgtcatccctgtcttctggggatttcgggtgtggacaccacacctattttgccttgaccgagaggcaggcagccttagacagtgcaccagtggcctgggattagaggaagcagattaagggctggagagtttgaggaagtgatggatgcaaatttaagtggattagcggagcctccttgaagaggtaacagtggagggtcaggtcctccagccttaaatcttggttgcagttcataaagttaaaactggctcattttgtcatcttttgaagtgatattaatagttgtttgattatgccaatctgaagttagcagaaattaatacttagatcattacagataaactactaaagtaaagagacatgtttttgctgttcttttggatgtatatattttactgggtttatccaaatatgtggaaattagttacctgtgtttgaaagaaacaatgttggggctgctttgagttctgcatgctaaagtctagcaatagagacagttatgggatcaggcatggtggtcttgctttgtctaaatgtgaacttggggtttttgtttctctaattaaacagttgcagttttacacagggtttgggaacttaccatcctgattaagagccacatgatgcagattaaagaacatctcattctttttgttctaaccagcttcatctgtattggaacttcattctagtatagcgctgggtgcatgcacacgcactgtctttagagacttttcccttgaattgtaatgttctattttggctaggtcgaaattgcttttagtattatatagtgttgagcgggtacaaaagtctatttttatagttatttgaagtgatggtgacagcttcagtggagatgtaaatggaatttattaggctaacctgctgctggagtttttgaattggaaatgcatgggggagggatgtagctcagtggtagagggtttgcctagtattcaagaggccctagattgaatactaccttctgtcaagaaagaaaaacaaacagcaacattgatttgaagttgccttgaaaaaaaattaaaaatacatgggtgagtgagtttgaggacagcctggtgtacatagtgaattcccagccagctagggctgcatagctagaccctgtcttggaaaggtggggtggggtgagggaatgagcagtgataagagtgcttgtgtgtttggaatgggtgttggggggccatcactgtaaggggtgcagttgcctgttttggattgtgaccctgagtgacaagccccgtgctactcgcttgcctctgctttcttagttgtcagacacagagtgtgctttcaggaaactgcccatcgatgggtaggaagcttggagttgttggtgaagcctagaacaataaggacacttttgaatccataccttaaaactagtgccacatgatggaaaatgtgattatttatttagggacaaggcctttgccaaggcttgtctagaatctgtggttctcctactttagtttcctgactattgggatatagcaggagccatcatacctaaatgctcttaagtgggaagagagcctcatacttttgaatagtgtgctgcacttcttcatgaggggtgtgtgtgtatgtgtttcttgaagcacacatttagttctataggtgcttgtgccaaaagctgccttcctgtttaaaacaattgagatcctgtagttgtatatatgctatgtctgagaaatgtttggaaaaatagacatgtgaaatatggtatataatatatatatatatatatatatatatatatatgacacagtctgtgatagagatgaacactgtcctggaggcagaacaaatttatttatgagataagtaagctgagtattcacttatttatagatggggttgtttttttgccttataattggaacttttaggttacttccagaaaagcaaaatattgacaggcatgttggtgcaagcttttaattcttggattcgagagacaggcaagtagtctctttattttgaggctaagttctaggccaggcaaagctacaaaatgagaatctcaaataataaaatctcactgggaagcaaagtcaaatactgagagtttttcttttcctttattaagataaccaacattacaggttcatataggatacaaacagagatatggcacataggttttttttcttccctcaaggtaaggtttagccttggctgtactggacctaaaaatacttttaggttattttatgtatttacaagtgtttttacctacatgtatatatgtgcaccgggtgcatgcttgttgcctttggaggtcagaagaaggcattaagtactctgagaatggagttacatagccaccatatgggtgctgggaagaccagctagtgctttttaccactgagtcatctctccagcctcttgttttaattttttattggtgcacatttcttcagcctcttgctttaatttttttagtggaacctttgtgtttctgggagtaatgtatatgaatgcagttttcctggaggccagaaggtggtgtcaggccccctggagctgaagttaaaggctgtgtgtgagctgatcaccttgggtgctgggaattaaatttaggtcctctgcaagagcactcactgggtgctcctaactgctgagccatcactctagcctcatagattagattttgagttcagtagtttgtatcttaaagattttttttttcttgagatgatagaatttagagtatggaaaaagaagaggaaaaaaaaatctttcaaaatagaaagagctaaacctttcagctttgggagattgggcggcttgccaaaagcaactgttgttgattgtttttaattctgcccctcccttccataaatgcagacagataaacaagatggtgggggggctgtgcgggcaacccactagagcagtgctcgcccagctcgtgttagactctgggttagatttctgcccagcaccacagaaaagaaaacgaaagggaaaacacagattggagtttggctcacttaaataagttcctcacttgttttttgttttgttttgttttttcgagacagggtttctctgtgtagccttggctgtcctggaactcactctgtagaccaggctggcctcgaactcagaaatccacctgcctctgcctcccaagtgctgggattaaaggcgtgtgccaccacgcccggctttccttgcttgtttttagtttgtataattctgcataaatgtaatgtgacgtctacttaaatgtttgtcttttaaagggcagggtctcacatcctaggctggcctgacacttggtatgacctatcctaagggaaacgttttttttttattcagtgcttctaggttgccagtgaatcaaaaagcagaactttcaggagcaaatgtcattgagagcaatgacaaacagtgtgagaggaggttgagaaactgcaggcttgtagtcagtcaggagaccttagaaagcacaccctgcgggtgtttgactgagttgatgaccctgggccttaggccctagggcgttactgccttacagcacagtgtagttaacatgctcacagtggtgactcatagtttaaattatttttaatagctttcaaagaagtatttttcaaactgcaggtcataattgattagattatgaaatctttttagttaaagtggtcttggttgaggtggagggtattcctcactgtgcagctctggttaggttgcagtttgctatgttgactaggctggcctctgacagatgtgcctgcctttgcctcccgagtgctggaattaaaggtacgagccagaatgtctggccctacatttttctgtttttgtaacccactgagtccagttggtgttgtctgttgagatgtaggtggatactggcttgctcttgtaaaggtcttgtgcaagtgattgtagctgcagtgatctctcgagtgcaaggaccatgttgtattcagaagacagcatttaaaacacttctccccatcccctgcttcttatcttctttctgcccctatttcatggtgttgcttgaagcctgagaccaggctctaggtgtcctacttaggccaagcactctgcactcacttattttcagcttctgaccaggtgtgagtctctgcagtaactactggccactgcagaatgaatatttgcctgtcaaagttgaaagtagctctgacttttgggtataagctgcagtatttagaaggcagttggactacaagtcacttggcagaacaacagttacattcttctgtatggctatgccctccttagccattggctttgatcaggtttacatcatcacagtgtgagtagttttctggtgcagtagttgaggctagagaccgattcacctcctcttaactcagttaagctgtgagggctgcagctttgttgtttgattgtatctcttggcacagagaaggtgattgtctgtttgtgtgaaggctcagtttctgtgtctagtaagaagtgtttcttagggttttcataaggagcacaagtagggtgggggtgtaggcctgtggtggagtgcatgtgctgccttcatccctggcaccccacacaaaaagtgaggcactgaattctgtttaccttctgtcttgtcaattccttacctggccctccaaaagtcacacagatttgaagctggaaggttctggtggtttttagtactcagttctatttacaggtgaagaaactaagggcaatgatatttgctaagtctcatgttgggctgcataactgatgttagaatgaggtccaccccctgatcccccaaacctaattaattaattaggctactccaggacctcacatgctaaacccgtgttccatccctcctcccatacatgtttcttagcatgtttcctgcttttcttgttctaagtattccagcttactccagagccttaggaatcattacagccacattctttggagagacttaagtggttttcattcttgcctgagtcaaatccagaccattctctttggtagtgagttcctccataggtggaacacacagtattcattcagcatgagctaaagtctgttgctcttgagtttgtcttcttttttttgtttgttttgtttttcaagatagggtttctctgtgtagccctggctgtcctggaactcactttgtagaccaggctggcctcgaactcagaaatccacttgcctctgcctcccaagtgctgggattaaaggtgtgcgccaccacgcccagctgagtttgtcttcttaaatgtagtcttcgtaggctttcccccagttcagttacacagtctcattgaaggagaaagggccagttcctgtgtccgtgcttctctgggaggtttagttcaggtggcgtcagtgcccagtgccttgcattaacagcctctccatctgggggcttcaacttaacagtgagtgctgagataagagcaaagtcacacctgcagtagaagagggttaatgccactaaccaaaacagataggtggctgtgccgctaatgagcaaatttaatcatacccacccaccactgttgttttaaaaatgtgtatgtgcttgaactcagcttattgtcagtgttgtcatactcaggtgcagccacagcacacactgacggtgttacagaagggagcagttactgctgcaaatgcaggtttttttttttttttttaaagattttttaaaatcttatttttattttttgagatgggcctatgaagtcctggctgtcttggaacctgttgtgtagaccaggctggcctcaaatttacagagatccttctactttctccctcagtgtaaaaagattaatttttttttttaaatttgtgtgtctgtttgggggtaccattagagcccagaagaggatgttgcatcacctggcagttgtgagtcctgggaattgaacccaggtcttttgcaagagcagctattgctcttaaactccggggtcatctttctcatcccaagagtaatatttagatagtgaacaatgaagtgattgatggtgcaaggcaggaccttggggaagttagttagagtcttagtttttgaggcagggtctctaactgtggttctcccagtaattttcccgtctcgccctcccaattacatatgtgagccaccatgcagatttagcttttaaatactactccccttgcctccagtaatggggatagggaccagagagtgtgctccaccatggagctgcactagcctccacctagcagagtaatttaaagtgaagacttttgtagctaacctgtgactgctgacctttgttacccccagcctagtgtgtgagctttctagttgtgattgccctgccagcaccttgtgtgctagccctgcccttatttagtaagggctgctgtgcagtggccttccagtgcatttctctggagagatggctgtgcttaagagtcttctagaggacctgggcttaacgcctagcacctacatggtggctgacaactgtctgtaattccagttccagaggatccaataccctcttctggcctacactgcatgcatgttgtgcacatatgtatatgatggtaaaaacacccatacacatgaaaataaaacttaaaaaaaatgtacttccctaatgcccagtaatagtgtgcatctttgtgcttatttataagcctttttttgtatcatctctgattgagaccagcttgcattagagcggggacacagccctataaaacttatgtggtttgtaactggttagttattgactggtcgtggtttcactgtggaaatcatgtgttaaagtcctctctgcgtagtttgtgtttcccagaaattacttgtgccaggcttgaggacagtagtggacaggtgagacccaggtctctcctttgagctcccacattggtgcagacagcagaagtagttcagatgcctacctgtctatggtattcaggcacttgagctagtgtgttgtcttattgaactcttagacagcttaggctacatagtgggggtctcactttaaacccctaagaagggtctagcatatggctctggtaaaggcacttaacctcaaagtttggcgatctgacttaaatctgtaaacccattcctgcaggttgctttctggcctctgtacacatactgtgacaagtatacttcccctcaataaatatatgtaataaatctatttttttgggggtgggggtgggctggagagatggaccagcagttaagagcactggctgcccttccagaagacacaggtttggttcccagcagccccacagtggttcacaactacctgtgactccagttccaggggatctcatatcttctgggcaatccacatacatggtgcacagacaaaacacccattcacataaatgtaattgagaaaaattttttgaaaaagttccttaaaatgttgaaaaataggaatggaagacagaataaattcttcatttacagatgcttttttttttttttaaagatttatttatttatttattttttatatgtaagtacactctagctgtcttcaggggcatcagatctcattatgggtggttgtgagccaccatgtggttgctgggatttgaacttaggaccttcggaagagcagttgggtgctcttacccactgagccatctcaccagcccgctttttttttttttaaagatttatttatttattatatgtaagtacactgtagctgtcttcagacactccagaagagggagccagatctcattacgggtggttgtgagccaccatgtggttgctgggatttgaactccggaccttcggaagagcagtcaggtgctcttacccactgagccatctcaccagcccctacagatactttttaaaaaagatttatttattttatgtatatatgagtacactgtagctatatagatggttgtgagcctttgtgtggttgttgggaattgaatttaggatctctgcttgcttggttggccccactcacttgctcaggcccaaagatttatttattattataaatatgtatactgtagcagggtgcaagacctcattacagattgttgtgagccactatgtggtagccaggatttgaactcaggacctacagaagaacagtcaatgctcttacccgctgagccatctctccagccccagatacggtttcatgtgtaaaagatcctaaggattccaccccagaatcctttggcactaaagaatattctcagcagagtgacaggatgtaaaattaacataaagatctgtagccttccctcctgtatactagtgacaaacttcttctcatgaagaagtcagggaaacttccattcaccttcaaaaacaaggtcggatctgtccagacctcactgttgatacttagtgcctgttttcttttctctgtaaatcatgttctaaagtgctcacgttgatgtctagcacgtatcagatgttcagatctttttttttttttgtgttttgttgttattgttgttttttggtttttcgagacagggtttctctgtgtagccctggctgtcctgacactcactctgtagaccaggctggcctcaaactcagaaattcgcctgcctctgccttccaagtgctgggattaaaggcgtgcaccaccacgcccggctgtttcactatctcttagagtacagttatatgtccccaccaagcatggattcattttgtacctttgtatcagttagaccaggattctttaaaaagatgaatggaggtttgcttctctttccaggttgtccagtgcagcagaaaaggagggtgggcacacaaatactgtgaactgttagccctgctgtggactggaatgacttccttttcagacagggtcttactttgtagctcgggctgtcctggaactcacaagtgctgggattaaaagcgtgtaccaccatacccagctgagtaagtcagcttcctccacacctctcttcacaagacaggttttctttgtgtagctctagctgtcctgagactctgcagtctagacaggcctcaaactgaggtcttccgtcttctgctgggattaagggtgtgcaccactatttcttgtcactcacatgtaaaagtcgagcaattaaaggacccttctgtacccttcagccagttgtagcagcagtcagtcttctacttcagctgtaattccatctgtcatggtgttttgaagggcattaatttttttcatgaggcataagctattacttatgaccagtgggtgaaacagtggaagagacttgaggttactacgtaaagcattcttggtgcaggctggaggagcaaatgcctccatcagccttcctgtctggagaagttgaaggaaaaccttatgctttataagtacagtatgggggctgatacccacacagggttagttcagttaaaaatggagctgtcagaaagtaaagccgtggaaagcctgcaaattggacaaaatagagtggaagaagagaactcactcccataagttgtcttctgaccaccacacatgtgccatggtaacccctccccctttatatactaaattcaaatgtgatttttttttttaaagggctgccgagatggctacagctggtaaggaaaggtgcttactgcctgaagccctgagtgggttccaggcaggacccacatggtgggagaggagaacagactgcaaattaccctctgacctccatgtgcacccctgtacacacgcatgaacacatatgagtgtttgatttttttcaagatggggtttctgtgaatagcctaggctctcttgggactcactctgtagatcaggctgctctcaaacttggagatcagcctgcttctgccttctgagtgctggggaagttaaataccatcccagctaaatacatgtatttaagaaaaaagaaataagggcttagtagttgagagcattggatgttcttccagaggactcaagttcaattccctggtgcccccatgacagctcacaactatctttaactaatgcattgtggtacacagatacaggcaaaacacccatatatatacaaaaagcagctcaggatgagagtaactgaaatgttcacgttacaatagctaagtcatgccagcctcgtctccagtactcactgctgcttgtgccagtggttgtcatatttattgaacagtgtgtggtgtgacatttccatcatcatggtttctgttgggacagcactgggtctgttcttatcccctgggtgtcaagaatcttgtgctatctgccccttgtaaatccttacctcctgatagaacccctttcctccccctgtgttatagatcttcacatggagagaacttgtttaataaagtttagttatagggttactctttaataataaactcaaagtgggaagagcaaagtgactcagctcaagttaactgaaagtgctctgattagatttttatgatgtactcagttgcttctttcctgtgtgtgtgtgttcatgtgtgtgtgggtacttgtgctcaggtgaaggtcacaacaatgttgagtgttgcttacattcttagagagcttagagacagggtctatcagtcattgtccattgtaggactgtcaccagcctgcatttcttcgctgctggcattaaagtcatgtcatcatgcccaactttttatatgggttctgatatccaaacttgagtcttttgcttgtagggtaagtactttataaatgagccacttccccagccctgcctttccttgtaactgctacatacagttttctttataggaaggagaatttgagtttgtcaatacttgtcatgattcacataaactatattgtagactttatttaacaattcaaaatatgctcagccctctacagatcagaaattcttagaagtcaggctggacctggttgtgcgtttatgtaatcctcgctcttgagaggctgaggcaggaggatcaccaaggctttaactactctttgggctgcatagtgagtttgaagcttctccagactaggtagtgagatccatgtcaaacagaaagtatgaaagacacctgtccttccaagtgcttggatgctgaggtcagatgagctgagctttgtgagtttgagatgaacctgagttacacagtaagccagccccgtgtccaaaaccatgcgtattgaatatgtgctgctctctaagcagtccagtgggagcactgcatagatttcaactgctaggtctacttaggcttgcttagagtgtatgggcaatgtctataggccatgtacaaatgcggccccattcactcccacttactcagtgcgtgcatgcatgagtgtgagtgtctaggttagaagtccaggtaggccctcgggtagttgatgtccccccccacccccagctgtaagttccagggagtaatcctgaccaaagtctgtcttggttagtatttaagaaacttgcttcaaatttggcttaaaaagaaaaagttggtacttaggaggtaaaggcaggtggatctctctgagttcgaagccagccttaatctacaaagcaagtgcccaggaggccaggattgatacacagagaaaccctgtcttggaaagaaaaaaagcatgattctgtagctcaggcaggtcttgcttgaactatggtcatgaagtgccgtatccaggagtacatggggtttcacatagctgagtgtaagtaggtagagcagcagagtctaaaggtttacactagcttgtattgtgcagttaccttttgaccttctgtgggtttgcatgtgtgattatgtgtgtatgtgttgggtttgggggttggtatttgtaggccagagactgatgtcagctgacattgtctctcattgagcctagagctaacaagttggctggtctggttggccagtgagttctggaatatgcctttctgcttcctcagtactgggtatacagattgatgccactatgcctggcttttggcttttatgtgggtgctaggtatcaggaatcaggctcccatatattttgtggcaagcactttatctagtgaaaacaaacctggatccttagctccttttttgtttgtgttggagtcaaggaggaagaataaattagcatttatttagtgtttgtttcaaccctaaagagtgtttgtttctggctcttttgggccaggtggtggcgcacgcctttaatcacagcactcgggaggcagaggcaggcagatctctgtgagttcgagaccagcctggtctacagagtttctggacagccagggatacacagagaaatcctgtctggagaaaacaacaaaaccaaacaaaagagattctactctgctagcaatatattaagaacttctttagtgcataatgtatgtgcacacatgtgtgctggcaggccacattcgatgtctagctctgtaactctgatttactcctttgaggcaagctctcaggaaacctggagctaggctggcagtggacaatacccagtgaccttcctcatagcgataggggttgcagctctgtagaaatcagtggtgtagattgaacagtaaatcagctccccaacgtcacaagttttgtgagaatgccttttttcgattaaaaaagtttttttagacggtctttgtatagccttggaacttattatgtagaacaggcagaactcagtttcccctacctacagagttccccttacctctgcctcctaaatgctgggaataatggtatatgccaccatgcctggctttttaggtttgctaatctgtgttgtcccacaagttttttagttatttcttgccttgtttcagatgcttgttgagctcctttagtgaatttctgtcagttacgcttttcatcttcaaatttttttttcctttttaaaaaattatgcatatttctgtgtatgcacacacacgtgtgtgtgtgtgtgtgagagcatgtgtgttaggctggggccctcagatgccagaaagatggtgttggatctcttggttggtggagttatagatggttgtgagttgccaagtaggttctgggaattgaactcagagccttttgaagggcagcttgtgctactgttgagccatcttttcagctcatgtattaagtcaatcaactaattcatttagtattcattttatggcagggttctcactatggttggcctggaaccaggctggccttgaacacacagagatcctctagactctgccttccaagcactggggttaagtgcatgaatacctgcttcccttactactttattgttattgtgtgtacgtttgtatgagtgagggcaggcaggtgctaagaagtgtttgaagtgattaggaccattggtagtaggttcttctgttcgtacctgggttcaaactcaggtcatcgggttcatgaggattgattttacccactggaccatctcactgccagggatttcttttcttttctttgcccaaggccatgcattcaccaggttagcactgtaacatagttgaagccctgctgtctccagtgacttctctttatgtagctttttgatgctgcatggtaagttagcacactgtccctgctgctggtcctgtttgtaatgactgcttttagttttggagtcagagtctttctattgtgtgtattcactgctgcctggtggctcttcctgcctcagcctcccaaactcaggcattgtagctgggggcggactgcctggctacttgtagttttactttttaaactgtattttgggtcacaagaagcagtttgtgttttttgtttttctgcttagttggtgaggtcatgatttcctgggctttgtaatttttttattgaaaaatggatattttagacaataccttgtagcaagtctggttctaggttggagctagggactggctgttactgtttacaatggacagcactacaggaggataaagaaattgtgctattagagagtcttataggactaacacagtgtaatacatagcatagggcccgtgaagttcttaggctgtctaatcttacgggatcactgtgcaaggcagatgttggcgcatggctggaggactagagagaagtcaggccacttcacacctggcttgcaggatgcacattaactgtcggttggatgatattattttttttataattttggtatgaatgtgtggatatgtatgcacagaagtgtgtagagggcagagctcgtccttgggtgctgccaccttgctgttttgaactgtctcagttaccttgtggttgagctaggctgtctctaccaccaactttttttttttaagatagggtctttctgcagaaccctggctgtcctgaaactcactctataggccaggctatataactcacacagatccaactgcctctgcctcctgagtttaggaaatcaaaggtgtgcagcatcacacccggcactacacccggctctctgcaccagtgctgggaactgagctcaggctctcatgtctgcttgtcacgccagcctgggctgtgaccttctccacatactgattggccaaatctgtaccattgaaggacagtttctgagttagaaattagtttttctaacttctcccccgttaccttcccacatgtgtccatcaaacctactagaattacatttcttttgagctcgccaattttctcttagtgccttgaccacaagctggatacttcttttattttttatttttttatttttggtttttcgagacagagattctccctgccaggtgctgggattaaagtctcgtgccaaagctgccacaaaaccaatttaatcgtcaattttctaaaaaaaaaaaaaaaaaaaaaagtcaaattatatgtatatggatgttttgccagcaagtatgtctatatactaagtgcatacttgttgctccctgaggtcagaggaaggtgttggatcctgtggtcctggagttacagttgtgagctgccatgtgcgagctgggtattgaactcagattctttgaaagaatagccagttctttcttttcaaagaaagatttattttatgtgttcaggtattttgtctgtatgcatctctgtgtataccacatgcatgttttcttaaccacgtagacccaattttaatcacttttttttttaatcactttttttttttttttttttttttggtttttcgagacagggtttctctgtgtagccctggctgtcctggaactcgctttgtagaccaggctggctttgaactcagaaatccacctgcctctgcctccccagtgctgggattaaaggcgtgcgccaccaagcccagctaattttaatcacttttaagttaaatatgacacttaaattatatcactgctatctgcattgattttaaacacagtttccatgttcagaaaggaagttttgtatacccaataagcagtggttcctcttttttcccctatatttggttctgggaatcattttgtctttgtgactgaatgtaaggccttgatgcagttgagcaatacttgttttctctgttagcttgtttactttagcatgcttcaagagtcctttgtgttgtaacataccagaccttcatggagttgtaaaatttatttgtgttattttttaacgttttgtgtgtgtgtctgtattgggtatgtggggtatatgcatgtgagtgtaggtgcccgtggtggccagaagagggcacttgccctggagctggagttataggcagctgtaagctgggaattgaactgagatcccatgtaagagcagtaagtattgagccatctctccagctctagagagtttatttctttggatgaatgcagattttgttgtaagccatcatgatgttgttatccatcagtgtgcatttgaatggtcttcccttggtgagtgacctgctggggactgtgtgtgactatctgctgactcacggcttcaggcaagggttttgatataagcccagaacctgaactactggatcttgtgatcattctatttagatttctcccaattactttagtttttatttatttttgatgtatataatgcctttattaatttctttgatgtacagagtgtcctttggttgtttcccactcccgaaactctcttacccgattccattcctgctaaacccctttccccaaccaaccccctcctgtttctgtgtctgtctgttgtttggtgtttggcgtccgctgacattgattagagttggttacgtgagtgtgggatgggagtcactcactgcactgcaggaaaggcactacagcactgaagagaagacctacctcctccagcctttcctcctccttcattcccagtaggtcccaaagagggctggcaccctaggatcccatgtcctgtctgcaggattataggcgtgcccagtcttggacaggtaacccaactgcttaagttcttgagcacaactgcaatgccatatgtacatactctggccttaaactccccgtgccctcccttgtctggtctgtctgaacgtggcggggtgactcacatggaagtcttgttttaggcctagcgctcattctctgcacttggactagttgttagcctctgaccaaggcagagagcagctctaacctataggaatgaacaaaaatatttagagataatttgataccacgtccacttagaaaacaacactagattgtctccgtatagcctgtaactctcctagttgtggacttttgcatagctttatggtatcagttaggcataagcattctcctgtggaacaggctttagataattagaaagtggtaatgccattattgccacataatgccacattacaacggtgtgtacaccttgactggtgctggttgtttggcttgtgtggttcacttcagggctaagattctgttcagattgatgtctttcttcccagcagcttgtggacaccttcaggtagccatgaaagccagccagcaggaggcccctgactgtcacagtgcgctctccttgtcctgcgtccaaaggctgtgctggatttgtctttattagcaaaagttgtactatctagttctggtggcaacagagtgattgagtgattgcaagaacctgaccgacaactctctctgtttggtgtgtgtgtgtgtgtgtgtgtgtgtgtgtgtgagagagagagagagacagacagacagacagacagacactgactgactgaccgactgaccgaccgaccatgcaagtctgtgacggtgctcaggccaggtctctcccaatcacttttttttttttaaagatttacttatttatatatgtaagtacactgtagctgtcttcaaacacaccagaagagggtgtcagatctcatatggatggttgtgagccaccatgtggttgctgggatttgaactcaggacctttggaagagcaatcaggtgctcttacctgctaagccatctcaccagccctctcccactcacttttatggattccagggatgcctttattcagtgagctagctgtctcaacaactgtgtttaatgatttaaggagtagccatgttagccggtggggcacactgtcttccctttctgtcaatggtacccaagattctgattctcccactgctctcacctacttgctaacttctggtattggttagagacggtgccagaaataattcatggcttcctgaaaggtaaatacttcaatccctgcactataagctgccataatgggtgtgaaatttaatttcctaaaaaactagtggtacaaagaatattttattttgccggacatggtggcgcatgcttttaatcccagcacttgggaggcagaggcaggcagatttctgagtttgaggccagcctggtctacagaatgagttccaggacagccagggctacacagagaaaacctgtctcaaaaaaccaaaagaaagaatttttttttttttgaggtagagtctcatggaatgcagactgctcttaaactcattatgaagcagggatgcacctcaattcctgattctcttactccatctcctgaatgctgagtttgcagggatatgccactatatgcagcttgaacatctttttatttttagattaatttcatgatttatttatgtgtatgggtgtctgcagaagccagtaaaggatgctgtttcccagaactggaattgtgagccatccggtgtgagtgctgggatctgttcccaccctggctctttggaagagcatgaggtgttcttaattcacctttgaacatctttttgcagatcattgattatctgggttttgttgttgttggttttggtttttgcttgcttttgcttttgctgtttttgaactaggttattggttttatttttgttaatgaattacagagttcacacacagctcctcctcaatattgaatattggcttaaagaggcatattgtcatgttgttgtcgaagctggtgctaaattactgagctcgagaggatcttcttggggctggagagatggctcagccagtaagagcactgactgctctttcagaggtcctgagttcaagtcccagcaacaaccatctatagtggaatctgatgcccttttctggtgtgtctgaagatagctacagtgtactcatttaaattaaataaataaatcttttttttttttttaaagaagagcacattctttgtcagtctcccaagcagctgagactgtatcatatatcactgccctaggctgactctccagttttacagtgcttttgatatcatatttaagaatccaaagtcacaaccattcgtagcagtcacaccttttaatcccagcactcaggaggcagaggcaggcagatctctgagtttgaggttatcctggtctacagagtgagttccaggacagccagggctacacagagaaatcctgtctcaaaaaacaaaaaacaaaaagcaaaacaaaacaaaacaaaaacccaaagaattcaaggtcacagtgctgttaaggagtttcatgattttagatttcctttttttcttttgaggctggcccaggacttaccatatactctagattgttcttaaattcaaagatctacttacctctgtttcccaagcactgggattaaaagcatgcactcccactcaggtttttctttttttcttttttctttgattcttatgagacagggtttctctgtgtatccctggctgtcctggcactcattttgtagaccaggctggccttgaactcagaaatctgcctgcctctgcttcccaaaggtttttctttttaaatgtgtgtgtgtgtgtgtgtgtgtgtgcatgggtgcccgatgaggctagaagtaggtgttgattccctgggaccagagttacagaaggttgagctgccatgtgggtgctggacctgggtcctctacaagaagaaatgctcttgactgctgtgcagtctccagtcccttccatctgtttgtttgtttattatttagtgtgagggcatgcaggcagacaggcacgtgcatggaaatgccatagagtgcacatggaggtcagagaacacctttctggagtggttttctgtccaccttgtgggtctgggggattgaactcaggttgtcagagttggcagcaggtgcttttaccatctgagccatccaacttccattttatttatttatttttaaagatttatttattttctgtatgagttcaattcccatgtggttgctgggaattgaactctggacctctggaagagcaactggtgctcttagccaccaatccatttctccagtcccccatttatttcctaaggaaatcttttttatgttgaatggatcacattttagatttagctgacaaatgcagcctaccagttggtggttagaatctttttttttttttttttttaaacaactcagccggatcctccaggaaaggtttttttttttttttttttttttttttaatttttttttttttaaagatttatttatttatatgtaagtacactgtagctgtcttcagacactccagaagagggagtcagatctcgttaggatggttgtgagccaccatgtggttgctgggatttgaactccagaccttcggaagagcagtcgggtgctcttactcactgagccatctcaccagcccggtggttagaatcttttgtgcttgtgtttttctgtcctgctagatatgggtgcttgatgggcgtcttttgtagcacttggtgtctttaagggatttgcaggaaagtagacagactggtatgatgacccttgaacacctgctacttcaacacttagcaagtcacactggtcttgcttcatataccatctatttccttcttcctgttactctgaagcagtgtagaagttattttacgtgcaggttttcttgtgttaatatagccagcattgtgtataggtgtgtgtgtgtgagtgcacatgtgagtatgtgcacaagattccaggtccgggaggtatgggtgccctggagctggtgagatggttgtgagtcacctgatgtgggtccagggaactgaactctgtcaccgtcaggaagagcttgaagagctcttaactggtaagctgtttttccagtcctctactctcctcctttcctttcctttcctttcctttcctttcctttcctttcctttcctttcctttcctttcctttcctttcctttcctttctttgagatagtgtaaaaaaattactgtggttgctctggaacttttatgtgtgtgtgtgcacgcgtgcgcacgcgcacactcagggtggtgggcacatacttgaggtcagaggatgatttggagtaagttctcttctatgatgtgggttctggggattgaactgaggtcatctgtaagtacctttgcctgctgagacattttgctactctgtctttaccatgtttgagagttagtgtttgttttagattgagcctctctggttgaaaaatgccagaaagttcacaggtcccaaagtctgaatgtgttggtgccagcatgatgctacaagtgggaagttctgcaccaagccaaaggcaatgtggtcagaatgtgtggccacttaaggttctgaatataggcaccttcagcttatgtgcacagcacaggtccatatgaaacattaaacttgggttccatcccaagatacctccttagtctaaagtccaaagaaattggaaattcaaaatccaaaacctttctattccaagtacttccaataaggcgtacttaagttaattctttgaattagatagatgattgggtctagtgctagtgaagaaacggagagctgggaatgtaactcagtgagcagatgctcgcataggatttaggaggagccggaatgtgtcccggctttggacgttgtggtgctcacctgtaatcccagcactcggatggtagaagcaggatcaagagtccagggttgcctctgctacataccgacttcagggccaacctgaggtacattagactcatgtttccacacccccaaagccccccaaactaaaagtaagtgggtcgagacccctatggacaggagtgccgaatgacccttcattcactttaaagaccattggaaaatatacatctacattactatttgtaacaactgcaaacttacagtagtaaattagtaaggataataattgtatgtttgggcatcagtacaacatgagaactgcattaaagggtggcaacattaggagtattgagaaacactggtctaggtgactggaagggggtttggtttgcagagcttcctttgctgcacctcagtgaccctctctgacatgtctgacttgtaggtcagttttggttggtgggtgtcagctctttgacactttgctgctgtgtaacaactgggactttttgttttccttttgaactgtctggttctgagtaaactgggttgcccagcaattgctatgtagtactcaaagctgtagttataggcagcaaagagatgatatgtgtgccttctgtcagggccttgttactttagtttactcatagctttgtggagaagtcacccatgttcagggatacttagtgctgtctcagcaatcaggaaaagaaaactctcagcaaggcacagctgtttcatcttttctgtgtctgttttcagacagggagtcactgtgtagcttaggctgtcctggaactcaccatgcagaccagcctggcctctgcttgaatgctaggattaaaggcatgtgccagtatgcctgactttcattttcatcttttaggaacattctgtgaataaatggaagtaagagctgagttaataattgaaaaggtggaggaaatcggtaagacatagttgtaaaattggacttttaaatttttttttgtttttttgagacagggtttctctgtctagctctggctgtcccagatcttgctctgtagaccaggctggcctcactcacaaaagatctgcctgcctctgccaacagtaaaattggatttaaaaaattttatttttactatttttaattttgtgtgtgtctgctttggataatgtgcatgttagtgtaggtgtccacagaagccacaggtgttagatgctcgtggagttgtagtcacaagtagttgtgagctgctcgatgtaggtgctgggcattgaaagagcatcttctcccagtgctcccagccactgagccttctcttgactatcaaccctcactttttgagacaaagtctcattgtgtagccttggctgtcctggggctcacttagaaaggccatccttgtcttgaactcagagatcctcctacttctacttcctggggctaatgacaccataccagtgaatatctttttctttttgatattataataggattaaataattccctttccttttccaacttctcctcccttgcctattctccttgctccatttcaaattcatggcctcttttttcattaattgttatttcgtgcatatatgtttatgagtatattcctaactataacctgatcagtctgtatgtttctcacatgtatgttttcagggctgaccgtttgtcattgataaccaattagtgtacactttcctggagaagactgtttctctcactcttagcattcctaagttgtctgtagttcttgtataagccttgtgggttttctcccatccactttgtgtctatcagtgttgtctcctctcatgttaaggcagccatgttggtgagacttttgacattactaggagacacaatctcacagcagactccctgattctttggttcttacagtctttctgctttctcttctgcaatgtttcctgagccttaggtatgggaatgttttgtagatgtatctactgggactaggctccataatgctgcattttgatgggttgaatgtttttgtaatggtccccctgtctgagaagtttcctggatgaggtgtgaggactacacttatctgtggatgtaaggacaattttctagagttaggggttctgtgattctctgaaatctgacttcacttgcactgagtagttggctaggtttccagtaccagggcatggtttttctctcactgagtggctcttaagtccaattagagagttgttggttactgccaaagaatgccactactgcaagccttagggtgatcacgtcatgccggtcattgtggttcataggcatcatagctgagtaggcttagagttagattgctggaagctcgcctcctttgtaagcttgcacagtggcaccgtgaaggctagtccttaaagggtgcaagcattcaggtcagttccagctcaggtgcttgtgtgtgggtcctgactctaaagtacatggtgtcttcagcagtagagacttacttatctcccacctctggggtcacccaagggcagtagcaataggctgtatgtttaggagtctgggtttttttttttttttttttttttttgttaggttgcctttgactcttggagaaagtgttgtcagctcagatgaaactctgtatgtaatcccctctctcttcctctgttagttaccctcttaaagattgccccacccctcaccatttccctattatgcccagctcttgttcccctcccccacaccctgccaatggtcattttttgtttgttagttttctgtttgttttttgaggcagggtctctgtagtcctgtctgtcctcagattccctatgctgatcaggctagcctgatgacagaccctcctacctgtgtattttaaacactgggattaaaggcggcttcagtgggttcctttttactgtcctggtttctgaagttactccagaatatactcacatgtgaagatttggaacctggcttctttagagataagattgcaggtgtgtgtcactgttcctgatttttgtgtggtactggggataaaaccagggccagttacagtctactaatcagcctgccttttaagtcttaattgtaaagtttgaagggtgccaggaagtttttgggttgtgattcccaagagtgaaaaagcataacccagaaacaaaagcttcgacgtagattaggtgcacctagttgcagcaggactttactgcatgtgaactgggttgggtggtttgggttaaggttcatgtccatcaactgtcaaacagtggttgatcagagtccagtgcataacattgtacttggttcctagcaagatctgtgacaagtgagaagtcaggttctttatgcttttggcccctccccctccccctccccctccccctcccctcccctcccctcttctcttctcttctttcttttcttttcttttcttttcttttcttttcttttcttttcttttcttttcttttcactccagactggaatgtacaggtcagaggactacctgtgagagatctctcctttaaattgtgggggtccagaggatggaatgcaggttgtcaggcttggttgcaagtgcctttaactgctgagccattctgccaggtgccaagaacattgcaaatgtgtgaatcttagggttctagaatttctgaaagaaatgagaggccatccagcctcagacagctttcctgagtgagtgaggtgaggaagacagttgaagggtggagaactaggtagtgggctgcctgggagttcctgctagtgtgctgccacgagttggcagccctgctctaggctttcacctgtttactgaattttcaaatttggactcaattggagtgtgtggctattgtattaaaatggcacttcaagggctggacagaagacccagtggttaagaacattggctgcttgtgtagaggacctgggtttgttttctagaactcacatggctgcttataattttctgtaactccagtttctggggatccagtgtgctgcttcaggccctgtgggtatcaagcatgaacaaggtgcacagacatacaggccggtgctcagctacatagaacatttaataacaaaaaaaccccagcaactttaaaatgggattccagatgtgttgttccacaacaggtggaacagaaatgtagaattccttgttatttgtgagaaactcaatctttccctttagaagaagccttcagttgtacaggtttaagacttgcaggttgaactaacagtgggtagaaaggatcgggagaaactgtaccagcaaggactgtgggtcatgtttttttccccttgtcattctctaaataatacagttgtgtatatgtaacatcttcaattgactagatattaaataataaatgaggtatgtgggtcatgcaaataccatataagtgatttgaacatcattgaattttggttctgatggaatcttgaaccaatcccctagagtacagaggaacaatggtgttatttattatgattcagttcacttgaaaatagtccacattacaaaggcaccatttgatagcagggttttatgttgtttaatgtgggttttgtttgtttctgcagtgctggaaactgagttgagggctcaaaaacaataagcaagcagactgtagtccccccccccccccagctgaggacctctgaggcaagcactctacctcgacctaaatctccaactcctaagcaagcatactctactgcaagctatattcctaacctggtagctgagttttgtgtactgtagcttttccaagggagaaggttggtaaggaaaagacttgttggacaattcctgaattttgacagtgtagcccaggctagcctcaagtaaggaagctactgcctcagctggggttacagctgtgaaccaccaggcctgcaatatcatgtaccaaatctgacagagacacatggtatttagaaataaaagtgtttagaactatctgatgtttaaacatttattttccgtgtgtgtgtgtgtgtgtgtgtgtgtgtgtctgtttgtgtgtttgtcccagcacacatgttgtagtggtcagaggacagattgtagaagttgggatgggaagttgtatggatgctggggattgaattcaagtcttaggcttgcagcaagtccctttaccttctgagccttctgacaaagccctgttttaacttatttttggtagttttttctgcagttataaacacctagctactttagagatggggagaatgagagctgattggggtgtcagttggccatgttgatcacaagtctgtgccaccactccacacttctagtgctgctcctttgcgtacccttgtccttcggtgcgtctggggtggggagacaaggtgtgctctacagccctggctgaactcacaccacagcatcccactgtctcagcctctgaagagtgagtgcagccacacaccactagacccggataggctgtctttactcttctgttatacatttgtttttgtttatttatatattagggtcaaggtctcgctcagctctggctgccctggaattcactgtgtagagcaggtgtccggcttccatctccctagagttgggatcaaatgcatgtggttctccacctgccttacctatttggttttgagaaaacagcagtcttgtcttttctttcctctttgtgggattctctcctggcctggtagcacacagccactgagatttcctgtcacactatattagatggcgccttcacagtcagcttggcctgtgtttgagtagaattataagattatattttatgtactcattaatttaaataaaataaaaattgaatctaataaaacaatgtttaatctttgcagaggtcggaacagtagcaaaggactgcctcagcctacggtgagtaactttaatgttatttagaaaaatacataaactgtgatttccaatcatagactcaataataaagcaaagaattagatttccagttgtactacagatagtctatagataagtcagtttaatacaaggttgttgattcgggaaggcctctgaaatgaacacttttctgttagtggtatttgtggcttacataatactgatgtcaagggttttgatgttccaggaccagagtttgcagagttctctgggctttttgttaggattaatgtgctgtctgttttaactcagtcatttttcctccttggaaaacaccttccggcctgccgttttccaacttttatcagtgaagttaaaatagacacaaagagcatgaaattccatataagaagccctcttttattggtgttttttttgcttggttggtgttttggttttctgagatagggttttctccatgttgttaactgttggaactcctgtagaccaggctagcctcaaactcagagaaatctgcctgcctctgcctcctgattgctgaaattaaaggtgtgaaccactactgcctacctgtttgttttttggacacagggtctactatgtagtcttggctggtctagtacttgctatgtagaccaggctagcctcaaaaactcagagagaactggttgtcactgagttttgaaacatggtctttatcgctcaggctatcttactgagagtcaggtggtcacaccctgtgtagtccagcctggaatggttttgttgccttgattcagacatgttgagagaagctggtcgtcgtgcaaggtgtaccctgctcagcacttctcttctttattaagatgtactgcccctctttaggacttgggttgggaggtctttcctcactaccctgctacccttggtgctcaggctctttaatgtgttagggaagcgctctgtcatccagctgctctccagcctggctgatgtttggttctgttgtggcttatatttagttttatttagtgtgggggtagtgggtgcaccatgatgcaggtgtaatagtcagaggacctccatgtgggggctgaactcaggacatcaggctcatgtgccttgacccactgaaccattttacgcgccccatctgagggagtgtgtgtgtgtgtgtgtgtgttttgagacaaggactaaatatgtatcctggctggcctgaaaacttcctgtgttgatcaggatgacctggaattcacttagatccacctgtttctgcttccagaatgttggaattaaaggtatttacaggcatgcctggctgtatcctacagccactactgcacaacccttctctacctggccgctagctgtgcaaagcacagaccctacaaatactgatttgtcttctccagaggtttttgctttctcacatggaggtctgagtctcctgtttttgtttgttttatgtcatagtaattattagttggaatggttggttgatttgtttaaaactctaccttttctcgtagggaaacaaaatgtgatagtaaccagctcttccttcgtgcattataatccaaatatcttagagctagacaaggttagcttttgtatataggcaagtacaaagatcagcttacaataatgtttattttttatacattctagatttcttttgatggaatctatgcaaacgtgaggatggttcatatacttacgtcagttgttgtaagttacctgattattggggataaactcctttggagtgggattttacatggagttaacatgtagataattcttaacctaatattattaggctatattaataaagatggttttaaaaacatactagaatgctattcagttaattacaactcacagtaaaaatactcttgttaactctgttgcatgtaaaatagtatttatagctgctggtcttttgtgtgatattttgttatctaatggtagtggggacagaagtgtgtagctttaggatacagtttatattcactttggagtttccagtcctgtaccaatagagaagtaggtataatgttaatgtgctgaaacttgatcattgccagtcaatatccaatactgaatgagaccttacagaaaatgaaagggagacacccactatgggaaccacactcttgactctgttaaattgtgtcttttagggatcgaaatgtgaagtacaagtgaaaaacggaggcatatatgaaggagtttttaaaacatacagtcctaaggtaattcttactaagtattagttcttcctgtagcttcctgagagtgtcttgtcattggttgagctgactgtggagttggcagttgagctcatgagataaaatacaaatgttttcctgattaccacgctctctacctgtttgataaccacttgtttttttttttttttttttttcctctctaataacaaattcttactatttaatgactggtttgtgaagctgaaaatattgtatgtgcttgtactggctggttttatgtgtcaatttgacacaggctggagttatcacagagaaaggagcttcagttggggaaatgcctccatgagatccagctgtaaggcattttctcaattagtgatcaagggggaaaggccccttgtgggtgggaccagctctgggcttggttctataagagagcaggctgagcaagccaggggaagcaagccagtaaagaacatccctccatggcttctgcatcagctcctgcttcctgacctacttgagttccagtcctgacttccttggtgatgagcagcaatgtggaagtgtaagccgaataaaccctttcctccccaacttgcttcttggtcatgatgtttgtgcaggaatagaaaccctgactaagacagtgcttatgattttcaaccattttcagatgttgtgagctagaagagggctgtacagagttatgtcttttttgttgttgttgttgttttgagacagggtttttctgtgtatccttggctgtcatggaagtcactctgtagaccaggctggcctcaaactcagaaatccacctgcctctgcctcccaagtgctgggattaaaggtatgcaccaccactgcccggcatatgtcttttttaacattagtgtgtttcagatccgctatagtaagatcagcttttagatttgagggtttttgtattgtaggtgcatgacaagtccaaacaggatgtgataatcaggttttcacaggatgcttttgtcataggcttaaaagtaaatggctttgatgagctaaatagctctataaatggctattaattaatttttttaatgttattaatttctttattttagtgtgacttggtacttgatgctgcacatgagaaaagtacagaatccagttcggggccaaaacgtgaagaaataatggagagtgttttgttcaaatgctcagacttcgttgtggtacagtttaaagatacagactccagttatgcacggagaggtaggcttgggtttccgaggtgaccctaaccctggtgtgattggctcactccaacagcaggtctccagcacccatacagacttggtttccctcacctcagacaggatcttgctatgtggctcaggctgaccttgagctcttggttctcctgcctttgcctcaagacctaggaatgagattttcttttcttttccttttctttattagtggtatttttctagaatagcttttttaataatctaataaaaatgtgagttttaatgaattttttgttagaaaatatttaaaacttaggtggaaaataacttggtaattttagaagcataaatatataattgcagtatgcattttttgaaaaacgtttttagatttatatatctgagcattttgtctgaatgtatttatgtgtaccatgggcatacctggtgctactgaggtcagaagatgtcagatccctaggaactggagtttctgatggttatgagttcactgtgtgggtgttcctatctactgagccatctctgcagccccagtgtgcatcctgctaacataatgttggccactactaggtaagccttgggttcaggagctccgtctgggctggcctgggctgggctgaacagtgggactatctcaaacaggatgctgttgtgggtgtggttgcgtattctgagaatttaaatgtacttacatttgtgtgtatatgtatatgcatgtgtcccctacctgttgcaagtgttgattaaagaggggcttgtgaacattggtctatcatctggagaggcacttgtagcactgggaaaggacctttgcaattaaaaaattcttgaatgatagattgtcactaactatagttgatttagattgtcattaattacagttgatttttgtgcttcaattggtgctaatggctatttgtgctttgtggttgaaatgtccatactgaaactctgaggcacaggaaatttcagtgctccctgactgtcctgtctctaatatctgcgtatctctgcctccagagtgttgggattaaagatgtgtcacaactaccagctgttaacagcctatctcaacacaattctctcattacttttgggggggggtgggcaggtagggggacaccttagatttataattccttagagagccttcagtcacccatgaaggagacagtgcttccttagttgttgcctttagagtcagtttcccttatgctttgaagtaccacatcatatatatttatggttttgaaagatttcatgtttggtcatcacacaagggggaagaccaaactgtgagtgaatatacagcactctgaggagaggattggctgtagcagaaacagcacagtttcagaaacagcagaaacacagtaccggctgttgtactgtgtttgcagcgtgcttgttttgggagtgctgtggaccaagcatagagtactgcatgcagttgatcatggttttgctggaccccaccaggtcccgttggagtttttacagtgctggaatgccccgtagccttacatgtgctaggtgaacctactactaagccccacccccaaccccacacaccaccccactctgccccagaacccccagagcaggtttcacggtgtagttctagctatcctggactcatcctggactcacagagatccacctgcatttgcctccagagtttggattagaggtgtgtgcactatgccctgtgtttgtttagttttattttattttagtttttaaagatttatttattaaagatttatttatttattttatgtatatgagtacactgtaggtatacagatatggttgtgagccatcatgtggttgctgggaattgaactcaggaccttggaaagagcagtcagtgctctttacctgctgagccatctcaccagcgcatttataatgtttagttttaaagacaggtcttcttgtagtcaggactggccccaacgattaaacttcttgtccacgctccccagtattggcattgcaggtgtgtgccagcatctcgggtcctgtatagtattagggattaaccctaggctctgtgagaatggatactttactgtgtattaatggttcaatttttactatgaagacatgagtattgacataatttcaagtagtttgtagaaaaccaggtatggcgggaggaggagacagaggcaggcagatctgagtttgaggccagtctggtcctcaaagtgagttccaggacagtcagagctcctcagagaaaccaaaagaaagaaagaaagaaaaagcccacagtggtttggttaattatacagttgtattttccagtgctgggaaagagtgtgcagggcctgtgcacactaaagagaatctctgcctgtcttcaagagtttaggatccttgagccagaattgtctgccaggtggacgtggaggccaggttacggttgggcaggtggtcttcttcagaagccatctgccctgttttgagacaaggtctctcgctaggactgggtgctcactgctgaggctagacagcaaacatgggtgaatgcctggctctgcctccccaggtcaggcattatgaacacacaaccatgtgccatctttggcttctgtggatgctggggattgaggctggctcttggatctatattcttgatgattaaaccttctcccaagccctcgagtcaggttctctgtgtatatccctgtgtgtgcaaatatcatatgttggttgaaattgctcttttttgtttgtttgtttttgttttcccaaggcaaagtttttctgtgtagctctggcttttctagaacgccctcagtaggccagtctcagagatctacctgcccctttctcctgagtgctgggattaccatgtttttgagagtgaagtgttgctgtgggaatattgcttatgtatagaagtttgacctactaatttggcatgtttcattaatcagataaattaaggtaatcagtgctgcattaaaaggaatggtctttaccttacggctttttagtctcctggagtgagaggagatctaatcagtgcttgcgagttttagttacttaggaacattcccaagacattagtcttcacttccttatattctgttttttgtttgtttgtttgtttgtttgttttgttttgttttgttttgttttttttaaagatttatttatttattataggtaagtacactgtagctgtcttcagacactccagaagagggcatcagatcttgttacagatggttgtgagccaccatgtggttgctgggatttgaactctggaccttcagaagagcagtcgggtgctcttacccactgagccatctcaccagcccttgttttgtttttcgagacagggtttgatagccctggctgtcctggaactcactttatagaccaggctggcctcgaactcagaaatctgcctgcctctgcttcccaagtgcttccacgcccagctgtttcgttatattctgatttacaaataatcagcttgtgaaatgatgagtattggctgctattccagaagacctggtcgtgattcctaactcctaaatggtggctcataactgcaaccccagtttcaggagacacagcaccctcttctagcctccttgaacactgtactcatatgatgcacagacataagcaagcaacaacccccacccccactccccagcacaaacataatacaccttgcttgagggtctgagcatgtgaaatagatgaggactgagccctaggcttctagtcctgaggaatggtgtggctctgagtcctgaaggacacacttactttctttgactcagtgcctttttttttttttttgtctcagttaaccattatcttgtaaagtttttgctgatacactaactaatatggtagaccttgatcagagatggactgataggtggtagtttgccatccaagcttgaaggaagtcaagttccccctggagcttctgctttcatcatttttctttaacaccactgtcagaatcttgaggttgttagctaggcatgatgggctcactgttattccagaattcaggaaggacactgaggcaggagactgccttgagttcaaggccagtgagatccatatctcaaacaaacaaatccaaccaaaccaaaacaaagaacctcagaaacaacaatagacagggctggagcaatagctctgtgcttaagagcactggctgctcttctggaggactagggttcagttctctttaccttactcacatgaaggcttgactctgcctataatttcacttccaggagaactgacaccctcaagggccaaccatgcacaggtaaacatatcagcaaaacatgcatgtgcatgtacacacacacacacacacacacacacaccctgagatagtttgaatatgcttggaccaggaagtagcaccatttggaggtgtggcctttttggaggaggtatgtcactgtgggtgtgggctttaagaccctcaacttagctgcctagaagccagtcttttcctaacatccttcagctgaagatgtagagctctcagctccttccacaccatgcctgcctggatgctgccttacttcctgctatgatgataatggactgaacctctgaatctttaagccggccctaattaaatgttgtccttataagagttgccttgggggctggtgagatggctcagcggttaagagtgctgactgctcttccaaaggtcctgagttcaaatcccagcaaccacatggtggctcacaaccatccataacgaaatctgatgcccttttctggtgtgtctgaagacagctacactgtacttacatataatatatgtaatacatatatatatatatatatatatatatatatatatatataaataaaaaagagttgccttggtcatggtgtgtgttcacagcagtgaaaccctaattaagcccccccccttaatttaaaaaataaaaaagcaaaaaactccagcaacagcactgggaaatgtgtttgagtggtatgcatgagatcttaggctcaggcacagtcatggagcaataaaggtgaagctggcagtattttaaaggctggttagatggctcaaaggtaaaaaaaaacatttgctgccaacccagaattcaatccctggaacacacatgcctgaggagagaaccaactctacaggttgtcctctaacgtctacatgcatgcacgcactctcccaacataagcaatacatgtaaaataaaaacaatttaaattcttgtttaaaagaagaaaaatcagctgagcatggtggttctcatttttagtccttgggaggcagaaacagaggcaggcagatctttctaagttcaaggccagcttggtccacatagtgagtttgaggatagctaaggttacatagtggagactctaaaaaaagtttttagttttaaattctgtgtattagtatatgggctgatggaaggtattggattcccctagacctggagttaaaggtggttgtgagccatccagtgtgtacaggtctctgtaacttttactcttaactgtcaagccattgtctctagtcctctcaggttagggcctgttgtaacgtgggcatgatttttatctacttgctccttggtaagaaaacagatgcatctgaggatgtggtcgagtagggacagagcccagttctttaaccctagctcatcacggctgtccttcacattgtacagtggtgatgagcactgtgggggcagctctttattaacattaaggatgtagtcacagataaagcagagggtaaagcatggtggcagacactgtgaccctaattttgcagaggctgtggcatatcagactacctactgagttctaggtcagattgttataaataacaagatcctgtttcaaaacaacaaccaccacctaaaacaaagccattgcaggcagtgtaagctacagattattgtatgattcaggtttcttttttttctttttcttcttttctttttgtgtctggcttttgcaggggaaaaaaatattttttgagacacgatctcactatgtagatcagacaggagtcaccactccctacgagctaaccctttaaagcaggtagcgtcagtccggcttttgaaccctgatacatactttattttgtcagtgagttgttttaaaatgtattgcatgtatgttcgcgtgtgtgcacgcataccttgatgtacaaggggagctcagggggagcttgtggaagcctgttctctcctcctaccatgtggttcctgaggtgcaaattcaggttgccaggttggtggccgtgcctttacctgctgagccttttctggcttgagttcagcaggtttgagaagtcactgttccagcactagggattcagaggccggtggatctgagttccaggacagccagggctacacacagaaaatccctgttttttcatacacatcttttttggtttttgagacagattttctctgtatagccctggctgtcctggaactcactctgtagaccaggcgtgcgccaccaccacctggctttcatacacatctttttttagaacttttttgtatacgttttgtttttctttctgtgtatgggtgttttgccttcattagtgtttgcaccacatgcatgcagtgcccatagggtccagaagatggtgttggataacctgaaactggagttcctgatggttgtggggcactatgtgagtgttgggtattgaacccaagtcttttagtagatcagccagtgttcttaaacactaaactgtcttcctcagcatctgattttaaaaataacttttataggggctggaaagatggcttagtggttaagagcattggctgctcttcccaagttcaattcccagcaaccacatggtggcttacaaccatctataatgtgatgtgatgccttcttctgatgtgcatgtgtacatgcagataaagcactcatacatagaataaatatatctttaaaaaatgattttaatgtgcatgggattttggtctgcatatctatgtctgtattgtttgtatgcttggagcccaagagaccaaaaccagggcatccaatgccctggagctagagttaaagacagttgttagctgccatcatgtactttgatttgaacaggggtcctcaggtagagccgctagtgctcttaactaaggcatctctccagccccatattcatctatctttacagatatatgggaggcttagaaatatccctatgttgtgggccaatatggtttatggactgagtaaaagtatcgtcgctagagtctgtctcgctcacacataggtagaacaggtcatgagattctttttcatgaatgtgtagaccagggtggctttgaaataacctctgaagaggaagctggtcctgaactcctgatcctcctgcctcacttgcccaggtggtgagaggacaggtgattgtcattcatcgttgctgaaaagatcagctcttaatggaaagttagctttttaaaaactaaaatgttataggtttgtttttatttttaaaggattttatacctttagtttaatttatgtgtattagtgtttggctgcatgtgtgtatgtttgtgtacagcgtccacagaggccagaggaggatgttggacccattggaactgaaggtagagcctgttaatagccctgaggttggtcctggaaaggcactgtggtcttttcttagtagggctctgctgcaaacagattggaagcacttgctttttctgtgtgcagcccggatgttcagccatagatggttttgtagcttctctgctgtattcagcagactagctgcaagtgtgagtttcagggatgcttgtcttcactcccaggtctccttaagaggagtttagggaccacaaaagctaagtcaggctcttaggtgagttctggggattcaaattcagatccgcatgtgtacatggcaagtattgttactcactgagccaaccttctagccctagagcactttgctgtgtgtactgtctgcatgtgcttgtgtgcctctgtatccgtgtaagtcagaagtagatgttggatgaggttttgtcccaacccctgttctttcttcttttagttttttgagatagattctctatgtcctagaacctatttagattaggctggccttgaactcagagatcttcctgcctctgccttctgaatgctgggattaaagacctgtggtttcctcagtcatgtcctatcttatttttggaacagggttgttactgaacctggagttcactgactggctagagtgggtggcctggggattttcctgtctgcatccctagcacggagatgacagttgtgcaggattttgtgggtcctagggttcaagctcaggtcctcctgcttgctctgtaagtgcttcaccaactgtgcctgcccctgccctattttttcctattttgcacctactttaagagagctggacgtggtggtacacgcctttaatcccagcactcgggaggcagaggcaggtggatttctgagttcgaggccagcctggtctacaaagtgagttccaggacagccagagctacacagagaaaccctgtctcgaaaaaccaaaaaaaaacaaaaaacgtgtgctgcttgtcttggaatgggacttgtgtctcttgctctgttctttccagtgtgtctggtctgcattttttttgttttgttcccccccccccccatcttcccattgtcccctccaaaggtcttcttacatgctaacctcagctgtcactgcctgtttcccagtccctagtgattagtggccttctgtttgtattgtatcctttgttttgatgacctccctgtgtgttggatctctatggcttctctcttgcttgtctctcatgctgccactttactgctttgtgcctttgtgtggtctcagccagcccggatagcaggtcagcagatggacacagtagtacacaaagttggattgtaacagcgatgtgtttcttcattcatgtgttggctgcagctgctgtcctgtagagctggaggtcagtcctaataacagacagcaggccttgcatatacacctcatctggcctttgacagggaagcttgacaacccttagcctaaatcattcttccttctgcattggagctatttagagctgatgtgttaaagagttacatgctgttctggtttggcgcaataaccgaacatgctttatgtctaatctcatattcagcgctgatgcttggctctagtattacatgtatcatctaattcagcttcttatttatgaccactccaattggaggcagcccaaagaatcacttgtgctttctagatgccacattcagtaagaagtaatgagcaagtggtttattgggagtgacctggagcaactacaactacctaccacttactcctcctctgcaaatttgattctcttttttctggctttgtggaaggaacaactaagcccccgtctccttgtgtttccccacagcattcctacccctcccgatctttcatttggcttttacttattttaagtgtgtgtagtgtgtgtctctgtcgtgtgtatgtgttactgtgtagagagaagaggacaacacctgtgggctctgggtgttggactttagcttgttagccgtaggccatcattttgtgcttttataccacttatttctggactccacactcttcctgctactgactccttcaattctgcctgaaaattattcaagtcccctttccctgaaacaagtcccttccccgttcctcatacatacagccctacttgaggctgtgtggagcttctatttcacaggtcctttggggctctttcagattcttatgccacatttgaggctttaccagtacctgcatctctttcataaacacatcatctctcttctgactcactcctggcttgtttcccctcctttacactgctctcttacatgcttgtgctcgcctggcctcgtggcttgtgtcagcccctccccagtgcccatgcaactactcttaggccaggttgtaggagggtgatggttttctcattataatctgaggtcttaaatctgagtaatgtctttattttttattttatttttcttagacaagaccttactctgtagcccaggctgtccttgaatttgctgtgaactctttgcagttctcttgtctgagcttcctcagtagtaggtttacaggcgtggaccacgatggcttttaatcttttaaaaagtctcattatctaccacagtgtcttgcttggcacgtagtacatgggctcataatgtctttgaattcatgatatttaatagcctaaaactttttttttgggggggggggttggtttttcgagacagggtttctctatatagccatggctgtcctggaactcactctgtagaccaggctggcctcgaactcagaaatccgcctgcctctgcctcccaagtgctgggattaaaggcgtgcgccaccacagcccggctatcctaaagcatttttaaaaaacatttattcatttattttatgtatgagtatactgtcactgtcttcagacacactacaagagggtatcagtttccattacaaatggttgtgagccaacatgtggttgctgggaattgaactcaggtggctcttaacccctgagccacctctccagcccatcctaaagcatttttaaagaactacaattctatatgaagtctaggaaattagggctggagagatgattcagtgacctttagtgcatgctggaccaccacaaagaccctggatctctacactgttataacaagccaggtgtggtccagctctgtagggtgagacaggaggattgctggagcttgctggcttccagcctagtggagaaaatgccaagtccaagatgtaggggagggactctgcgtcagaggaggaggctgcatgacaggatgagtgacactgcactgcctgctttccagtccagatcgtgcacctccttacacgtgctcacacacgtgtacccctaaagtaaatgaaactagccaaagatggctacagatgtcttgctgtgttcctgtccctctgcttatggctctggacagctcggcgagatgtagagattcttaggagttcttttgagaacttctgtgaagcttcctcactctagggaactacagacaaacttgtgcgtttaaataccacttgagacaaatacagacgcttgtggaaatagacacacaaatggaaaacatttgctaaagttactttcagacattcagaaaatacagaacttatcttctgtattttttatacatatgcccacatgttgtttgacagccttctggggtgatataacacatcaacagaccatgacaatgactgtcgcagcactgtgaagttcatgaactaatgagttttattgagattcctagaggtgagaagttacttaaaaggattaggggtaactcagaaaagcagctctacactaacatgcgtaataacatgaatataataacatgtgaatactgcagccttggagccttctgtgtagtttgcaggcaggtttgcttggctgtctctgtccttagcagctttttacttctttttatactgttagtagggcccccaggaatctcacaagtcctcatccctgtttctccccccatgtataaccatccgtagtaaatgttctatctccaaagagacaccatgaccacggcagctcttataaaagaaagtgaacatttaattggagtttgcttatagcttcagaagtttagtcgattatcatcatggtggggaacatggcaacactcaggcagacctggtgccaaagaagtagccaagagttctgctttaggatccagtagcagcaggtgagggctgacactaggcctggcttgggcccatccccacatgaccttctgttcactggtctgtgagaaccccctttcctctggaggggatgttggcaggtgtagaaaacctgctgtataacagcaggtcagagcattaaaaacgagaaaggagttttgctgggcctggtagtgcctacccagaaggcacagaaggcgaggcaggaggattaagactgctatttagtgctagtaaaagtaaggtgccctgattgatttcttacgtattttacttttaaaaagttacatttgtctattatatatgtgggccttggtgctgtggtatctgtatagatgtcagagacagcctatgggaaccaattctctattgtgttagtgccctgcagcaataaaagtttcattgtgtggtggtcggtagctttacactgagccattttaccgggctgtgtgtctgtgtggtcccccaccccatgggctctcctgtattcccgctgctggctctgtagccaaggatgatcctgactccagtccatggacgtgcgggagtccagccgagggctggctgtatggcagacaggactttgctagctcagcactaactgctttcctttccctgactgggtcagtgcccagcttgcaaaaatcaatgctaaataaatacaaacattattaaagtagatttgatcatttgaagattttgtaatgctttttgtggtcggttcttagtatgctgatgtgatgctaaaggctctgcacttccatgtattaacaaagagaagacatgttttctttcatagcaacagggaggacatgccttcccagacatgaaagtgctgtagagttcagtgtttgtagtgacagagaatagaaaaccccagacagactcatgtctaagtgtgcatgtgcgcagtaaaggtaaggcttccattgagagaagcatgccattggccagcagcattggatagcatttaggagaggttggctttctgtttcccacgagttcagcactgtatagtgtaaaacagcagtgagctgcaaacgtgtgctgctacatccctggcatttgagttatctgagagagaagagccttatgggcatgtgcttggttggttgacatgttgggaagagagacctttagtgaagaggatctttcatcactttggcccttggacaactctataggactttttggttttttaattttttattgctgattggaggaggatcaatccctctctgtggaggtatgagctctggacaggaggctgggctatctatgtaagagtggcagactgtaaggctggaagcaaaacagccagcagcattccaccaaggtgtctgcttcaagctccttctttgtattcctacctcaactttcctatgatctatgggaggaaccaagacagtttgaagtagcaaattgtattttttaaagaaacagcatgttggtgtttgctcttttatattgattgttttcagacactgtataaaatggttcctacatcaagtcctaaaaggaataatatgggagaagataagatgtctcagttgatgaaggcgtacagtaccaagcctggtgacctgagttctgtccctgagacctacatggtagagagagaggtgtgctccagggcacatatgttcacctgctctcactgctgcccgatacatgcataagcttaaagcttattttattttattgcggataaaatgatttggttggtgccagttgttttgctttgcttcttctggccctaagcttataggatcttctgttctttaacccagtcaggtggcagctgcagaaggccctccgtttccttagagcacagtgcctgaggtgcttcctcaagctgcctaccctgtgctcggagtgcagtgctgctgctgtcgagctatgcctttttaaaaacatgttttttatttttagatttattgtgtataagtttgtgctactgtatgaggccagtgtggactctcctggagctagagttagaggaggttgtgaaactgtcagatgtggccgtgggaaccgagctcatgccctgtgaaagtgtatcaggtattctccactactgaaccgtgtccccagctcttggccctgaactttggaagctaatatcccacaccaaggtctgtgtctccaccaatccttctggtcgcctctttcctagactgcttatctcttcattgtgtgatcacaataaaagaataagtgttgttctggccaatattatcatattgtaatcaaaggatcaaggcatttttctcctggttgtgaatggatcttggcaagagaaggatccactatttctcctcatggggtttggttagtttcctgttgccatgatgaacctgatagaaatggcttacagggggattatttagcctgagtcgtggcttcagagcctgtagaatatggttctctggctctgtgaactgtgaagagcagtaggttgtatggtggttgagggtttcacctcaaggaggcagaaggaaagcaaaaggcgccaaccagggcaacatgtaggtcaaggacagaagccccatgacctgctaccgctaattaagcaccacctccaggtaactccaccatactgtaaataacatcagggctaactggttaacttctagccctcaggagagaactctctggaaatggacttgcagacctaacagagatacttactaaccacaatggtatttcttagtttagtcagttcattatggagacaaaccacggtgctgtcagaagctatcccatttgagtctgtacacctataatcctgtctatttctgcatgccaaaaatgtcatttccccagtgttgtttgtgtttgctacaaaatttgctttcatgtgttgtttatcattttaagaaattagtcatttgaagcttgttgttttgaagtttttgttttgggaacaggtctgtgctgtgtagattagggtgacctagggctgcagttccttccctgtcatctgcccggtgctgggacatgctcactgccatccatgccctgcaattagtcattcttgcttcctccccctcccctcccctcccctcccctccccttccctcctctcttctcttcttgaaattagatattgtgttatgtatgtaagtatactatagctgtcttcagacacaccagaagagggcatcagatcccatcacagatggttgtgagccaccatatagttgctgggaattgacctcagaacctctagaggagcagtcagtgctcttaaccatctcagtgcactgagctatctctccagcccctcttgtttgagttttgattctccagacagcgtttctgtggcttttctgaattcactctgtacaccatgctggccttgaacttgaccagctttttaaattaacttttttttactttagcaaaaattataccccccccccccccaaaagtcccatccacttgtttttctttatttccttacctatgaaagtttagcctatgaaggtttttattcctgatcttttgtggtttgtatgcttacagttttatttatgtgagacaaggtctagccctgactggcctggaacatgatgctgtgtaggccaggctgcactcaaactcacagaggtctctctgtctctgcctctctgcctcccaagtgctggaattaaaggtatgtgtcatcatacctaggtattttaccttatcttattggatttgattttgagatagggtctgtgtgcagtcccagctgtcctggaactcactaagtagattagactgagctgtttttatatatgcatatatggtttatttgtgtctgctacatgtgtagatttccagaggccagaagagagcattggctcccctggagctgaagttacaagaggttgtgggtttctttgcgggtactgggatctgaacttggtcttttggaagagaaacactcttaccagctgagcctcactctcaaaccccagaactgattgagcttgtggcagattttaatttgactgttgatgtggatatgtcttgtcaagcatcctctgctatagaaactgtcctcctagggaagtgacttagtgcctttcctgacagtgaggctaccagatgtcaagatgtgtggcagctgcggaggagaccgagtggttaagagcacctgctcaatcttgcagaaaacccagatctgttcccagtattcaaaaggagttcatacctctctaactccagttcaggggagctgatgtcctcttctgggctctgcaggtgctgcaggcatatatgagggcaaaacactcaaacatataaaattaaatctttaaaaaatgatatatgaatttctgattatattttgttgcgccttgtgcctgattctgttcagagctgtggtgtcttgttactgtgccagtgtagtaagttgaaatttttatggatttattttacatgaatgagtttgcctgggtgtgcagaccttgtgttcttgaggtcagaagagggcattgatatctggaactggttatagatgacagtctctttgtgggtgctgggattgaacccaggcctctgaaagagaaacaagcactactcctcagaatagatactctcaagctttttgagatagggtctcactgtgtagctctggctggccttgaattcaaatcgttcctccactaccaccaccataccatgaaggcagtggctcccacacctggcaaggacttcatattatgctcttctttagggatagacttccagaatgacttctagtcaacattccttcccgtgactcttcaaccttgatggccttcttgagcttatctcatctgccctgtttatccttgggtttgctttctacaaaagcccatgcaccgcagagggtgctagacgcgctttcagaacccagaaccagatcacacctttgctcttcagtcctgagctttttgctcacccaagttgctcttcagctggatactttactattagtttgctgggctgcacttgatgcagttgacatggtttctgtgggctgaggagaatgagggtttcttggagccccaggcagctttttctcatgactcctggggcacttgaggagatggttggagaagccgtggtctgctgggggcgtagcaagctttcctaggctgtaacagtgtccctctctagtccttattgcataatcattcctcaagatgagttcctaatcctgggaaccaagccaatagcagtcatctattatttttttataggcatatcgatacagacaaagattacatttttgtttaacattcagttctgggaaacaaaagatatctaaaaatgatatttttcagtagcatgatatttaagcatatttttgtggttgcagatcatttctagtcattttgatttttagaaggactgatttaatgttttgatctctttgtttttgcttgcaggcctctatatacagcatgtgcatggctagtacccatagaggccagcagagggggttagatcccctaactggagttaacataagattgtgagctgccccatgagtgatgggactcaaacccacatcctctgcaagaacagtgcatgctcctaactgctgagccatctctccttccccataatacactttagacaataaaaactctaaagcgttagatttgacaagtaataggctcagtacatgtaattctttctttactgagaagcattctagcataagtaaagtgtgcctagcgatttgagctctttgacttttcctttgtgtgtgtatatatatatatatatatatatatatatatatatatatatatatatatgtgtgtgtgtgtgtgtgtgtgtgtgtgtatatatgttgtgggggtggcctttaaactacaaccgtgcattcagtgcatccactgtgaaggaatgcatgaaagtagcctagctgcttctttgtttggagtcctaggattacaagatcccctcaagatcattcatatgcaaccccttctagagcatgctggggaaatgaccctttggcagccaccaacgggtgggttagacatcccctgtaggtgtcagtgttctggaggcagttagatgtgattctctgaaagtgtagcatcctgttatattatggttggaagtttgttgtcttgactttctgtttatacttggtatttgggggtggctcttcgtttctgctatttgctgtggtgcctgtcctgctccacagaaagttttgacacattggtgtccacagtatttgttagggtgatgtctctatctgctagtgagtccaggtagccattcagtttcctttagtgcaaagatctttaaagatgagctgggactcagtgttgttcccagagaggcctggcttcctgtcccacctggcctcagtgagagagtcagagatagcctggggctagcttggttttattttgataccttagttctttctgtttctctgctttaaagtcttcagttgaagaaacaactctggtctctgatgggactccccagcgccttgcattttgtggttatggttgatgatgatctctacagtgtgtaaaacaaactgtttcctgacatttaatcaggcaaatagtaaatgtgttatgttcagtgggcttagtcattcagaatggatggtatttgctattggcctttttcccccagacaaaacaggtgctgttatgcagaacagagctgtgtgtttctggcatgcagtgtgttgtgcttagggacttgatgcatttgatgcaagttttcagtgacatgtgtggcagagatcgggttttccatataacctgtttgttttgtttccagcagagcagtatctcactaggcacattacccttcaacattttgtgtagcagcttacacaggaccacagccatcatgttcctcacgacactgctcttctcttctcctgccctcctgtgtttcttttcttggttgtttttgttttatttttaagatgtatttgattcttttatgtaatgaattaattaatcggttaatttaatgtatgcgagtatactgtacctgtcttcagacacaccagaagagggcatcagatcccattacaggtggttgtgagccaccatgtggttgctgggaattgaactcaggacttttggaagagcaagtcagtgctcttaaccactaagccatctctccagccccaaagttaacccattaaacctcacccacccaaggaccaggtaacttcctagaattctggggattgtacagatggaaaagtatggtggccatatgggtgtgtcctttaaaattcctacaacatatgtctcagggccactcaactgggaattataaggagagatcctgaccaaagtcattcctggtcagtatttgaatacatttggctcaaaagtgtggaactggtttcctattttgaatctcaaaattaacatttggaggcctcagtgagataggcctttccatcccaaattctggagctaggatttcactctggctcccccacccccatgttcatgctgggaaaaatgaaatagcattttttatggtaattcatagaagccaagggagacatcccacaacagtttctgtggccttggctgttctagaattagcttcgtagaccaggttagcatcaaactcagattttgcctgcctttgcctccatctcctgagtgctcgggttaacatcatgtcccaccactgcctggctttattttaaattatatatatgtgtggtatgtgcacgtgctcatgagtgcccacgtagaccataagttacattaacagatggttgtaggccatccaacattggtgttgggaattgggtttgggttatctggaagagcagtatgtgctgctgttaaaatgcttgaggcatctctagtcccttgaagatttgcttgtttgggggacaagatcttacttatcatgggctaactcggaagtttttatgtagctgagcatgtcttttaacttccgtttctcctgtcctcaccggatacatactggggatggcagacattgtgctgttatgcccagtgtacttcagtattgtgtaacagaaaagagagatcatttgtatgccattacatttgcctgtgtagtgtgtatacttgtgatgcgggggcttgaaaatagagccgtgtgcatgacagccatgcattctgcccctcagccatccccgattcctgtctgttctgtttaatctggtcctccttcagcttctgccctttgctgtttttaattaaccaggcatggtggcacaggcacactgaggcaggagaattgccatatgttacagcttagtctaggtctgctgagtttgaggtcattctaggctgtttcagaatgagctccaatctcaagaacaaactcagctggatggtagtagcacataccataaataccaacacttagtaggcaagaggcagtcggatctctgggttcaaggccagcctggtcctcagatcaagttactagctacacagagaaaccctatgtctagagagaggaaaaacacccaaaacacactcctgattctatgtgagcactcagttaccgtgtgtgaattccctttctttatgtatgtagtcggggaaatgaagcaggctgaataaaaatgccctcttagcagcctacagtgtagacagcaagaggagcctgatctgggaattcttttggcccactcactcctccatagctatctcctgaataggctgctatcttgtcttgctcaggatcagaacacaaaacagtaggtaaaagtttgtgggttggtttttgtaggtttttttttttgttttgttttgttttgttttttttttttttttggtcgcttctcccctccctgcccccaaccctcacagtggtatctttgtgtggcgtgaaatactaaaaaatgcaacccatgatattccagctggtcccagccggccttgttctcatctttggcaaactctctgacgtggagctctgaaatcttgccacaggactcgctaagttcccactgggtcactaccacgccagccccgctcttccaaattcccatggccttttggggtgcacttcttgcaaacccatgctttgcagccctacctttctttctggaacccagttgagttgccatgtgaaggaaaagaccacacaaacttagttcaggatcaacagttaattcagttgctgggcacaacaactagaatcctaatcttgtaagccatattaaatataaatcaccatctaaatgggggccacttgtagctacatcttgtcctcccactgtccctgtccaaaagccctcttgcggaagctgaattaacatcaaaatacaaacagcatcagggctatccacaacacttttgagaggagcttttggttcatcccacacctgctgacatttttatacacctcccccataaacaagaaaaggtccatttaggccagctgtggtagacatctatattccctaagtctgaagatggataatcatagagttataggctgttgtaccagactatttatttgacagagttctgtctccaaaaaaggattataatttgtgtggttgtgagacagtcttctatgcttttaactttcatgataatgtcttcctttcctatgttcctttttctgtctgtgtctgtctgccctccttctccctctctctctcccttcctctttctttttaaggtaggatcttattgtatgtattgtacaatagtgaacctcctacccaagcctcccagtttgggggctcagctcccagctttgagttctaacttcctgaattagaatgttagtttttttatactgagtttgtgtttaaattcctgcttttgcaacactggtgattaacatgttaacaggattttgtttttgaacattgttgcagcttttgtgtagagatgagtctgaatgactctaagtgccctgtaatctacttctgtggttttggttccttgtagttgaagagatcccaggatcctaacaaatacttgtttgccattattatcggaatcactcctctttctgaaggtatacacaagtaaattcaattgattgttcaatcagactcactctctttttttttttttttaagaataaatttatttcatgaatacgagtacactatagctgtcttcagacacaccagaagaggccatcagatcccaaccccagtaaaccttttaaaattgatttaccggctggagaggtggctcagtagttaagagcactgactgctcttccagaggtcctgagttcaattcccagcaaccacgtggtggctcccaaccatctgtaatgggatctgacaccctcttctggggtgtctgaagacagctacagtgtacttatatacatgaaataagttttaaaaaagttttgctcaggtgtatcttgatctgagtgcaggtgtctgcaggaatcagaagaggaggtcagatctcctggacttgacgagtctcccagtataggtgctgagagccaaagctggatcctgcaagagcagggccgtctctccagctccacaacttaatttttaccaatagttgatagagttgtcagatgatgtaggatagggcttgagtcaagctagcaggaaataatgaggctgactagttagggctgtttgtagcttccagttgctggggggttgggagatgagcaacatcttatgctctgcaaaaagctttatcctgagcgcagttccattctactgttgctcagagggctccctgtgtgcactctttgtccttatcattgatagtagcaggagacatctgctggtgaggcctcgtatgtatggtgcacattcatgtctatttgcccctaatttgggaagacaggtggttagggctacctagccgtgcagatgtgtgaccatgagcattcctgatgctgttcaactctttaactcccagctgtagagcagtaaggtcagctgagcatgtaagtgccaaacattttcataagctagactcctcaagggatattacaaggagtgggattgggtgcatgtcagtgagcctgggctgctagaagtacccatgaggctgccggagacagcagcactgcagaatctgaacagcttcttgtttggttttctgagacagggtttctgtatgtagctctggctgtcctagaacttgctctattgaccaggttagccttgaactcagagatctgcctgcctctgcctcctgagtgctgggactaaaattgtgagacaccactgtcctgcttgaatctgaacttcttaaggtgatattgaggaactgttttggaaagggacagatcacttaggaatggtcttacttattaatatctgtattgtcaggaggtacatttggatactgcacagagacctaagcttagaaagttaaaaccattctgcctagtcacacatggtgggcacttgctagctatcccagaactgagaaggtacatgcaggaaggagtgctacaagttcaaggctagtctgggctacatatttcaggctatctagggctatatatagtaagactgcctcaacacaaaaacaagacaactaatgtatttggctatagatacttagtgtcgcctgcctgttgttcttcacagatgcttttactgactctgctctcagcgcaaaggtgaatggtgagcacaaggagaaggacctggagccctgggatgcaggggagctcacggccagcgaggagctggagctggagaatgatgtggtgagtgccctctgcaccccagcactcagtgtgaacgtggggagaactccacacgtgcagagcagcactttctctgggcagttgctaaagagggagagaaggaaagaatttctccctgactggagtcggagcactcttaaattacttctttgtgatgatcagttacttcagggaactgatagttacaagatggcagcctgtttcgttttgaataccaaccgaaagcaggtagtgtttaattgtatgttgacttccttagaaatgaggtcaaggtggataaagagttatcattaagatagtttcaggttgggtgtgacaactgtgatgtcagcactcagtcgtagaagaataagggtttaagagttcaagatcagcctccactaaagagaaagtttgaggctagcctgagctttataaagaccctgactcaaaacaaaacaccaaaacaccaacaacaacaacaaaaaacccaagtcagctaggtgttacctgccttaataccaggaggcagagacagacaggtctacatagcaagtttaaggctagctacggctacacagccagatagtggatgaaaaatagcaatcataaaagataaatacttaggttacattaaaattaaagcctggcctaaatttcttgaacttaaaattagtatcattctctataggaacttgcttctaaaatgaccatttgtcatacattattttttgttttctcttggcaccccatctacaagtcaaatattagtaaactttgatggagcatagagcttattatattttctttaattaaaaattttattttatgcatatgggcatattgtatgtatgcctgtgacctattcctaatgccttgagaggccagaggagtgcgtcagacccctagagctggagttacagacaactgtgagtttccatgtgggctaggacttgtactgggtcctttggaaaagcagccagtgcttataaccgttgagccctctcctcagtccccttgaacatagagatttaatgttatggagcgtcatgaataagactgagtgagcaaagaaccttctgttgtgcacagacaaccaagtttactatttaagacatagtaagggaggaaatagtatctcttctctaagagctcagccccctcaaagaatagaactgccctgggaagttgtcagtgttaatcttcaaagtttatttatttatttttttgttttgttttttttgtttgtttgtttgttttgagacacagggtttctctgtgtagtccaggctgtcctggaactcactttgtagaccaggctggcctcgaactcagaaattcgcctgcctcagcctcccaagtgctaggattaaaggtgtgcgccaccatgcccggcttaatcttcaaagtttaaagaatctcctaaacttaatataatggttttcatgtgtgcatcactgcaccattgtgatgggaaacaggtacagtttttactttattagtggagagataagttctttaatggtttgagggggagactcaggcagtttatcagtatttaaaatgtaagtgtggtttgttcccaattagtcttactacaaagaatttaccttagctaggcagttgggttctgagttcaaggacaacctgatctacatagcaagttctaggccttcctagggtatcctgtgaaaccctgttgtttgttttttgtttttgtttttaagacttatttatatcatttattttatgtatgtgagtacactgtaactgtcttcagacataccagaagagggcatcagatggttgtgagccaccatgtggttgctgggaattgaattcagggcctctggaagagcagtcaatgctcttaaccactgagccatctctccaacaccccgtttttggtttttcgagacctgtagtagccctggctgtcctggaactcactctgtagaccaggctggcctcaaactcagaaatccgcctgcctctgcctcccaagtgctgggattaaaggtattggttaccacctcctggcttgagacccctttttaaggggaaatatataaatttaaaatagaaaagaatttatcttagtcctaatatacctataatacgtgtaaaataattatatgtcatgatattttttcaacattatttctattagtagtttcccatcagaaaagcacaatgctgatacttaatagacatttgtgtacctaccaataaccagtattcaaataggaaagatacctcatatatttattaacaggaaaagcaagctgcagagtagcaataaaagtacatagtgtgtgtgagagagagagagagtgtgtgaatattcagtttatgtgcaccataaaatgttgcccagagctggcctcggttacggggagttgagatctgccttgcaggtgctgctgctgtacatcctttttctaagttctaagagcatactttttgttgttgttttattggcggtggtggtttttttgtttgtgtgcttgttttgaaacagggtttttctctgtagacctggctgtcctggaacttgctctgtatacctggctatttgaactcagatcttcctgcctctacctcgtgagtactggggttaaaggcccgtgccaccatgctgagctctgtagtaacagctcttaaccttggaaccattggcctaagcccgtgaaactcctcatagtggttattcctatgccctggaacttgaaatcattagttttggtggatatttttctgttcagtgtgatggatcactcaaattactgaagttagagtcttccaattaaaaaaattatgtatctatctatgtatccattgcgagggagagtgtgtatgtatcatggcttgcatgtagattctaaggacagcttgtaggagttgctgctcttcctccaccatatcttccagaaaagtccatgtcctctagaaaagtaatatgttttcttgattactgagccctcaacctgtcagactttaaaaggagagtttgatgtgagatttgctggggaccaggagttgcctcacaaactacatgaacactgatatcagtcagtcaggaatagtttattgactgttccccccaggactgatgcatgagggctgcagatgtgacagtgagtcagattctgtagggctttcaagcctgaaatctacaaacatgtgtgccatgatcttctgccagtcaggatgtggggatagggataggcacttccttaggagcttgtctttggtgtacatttactctgcttccattggttggtgtattcagcagtggcaaggtcttgccttgcctaacactcatgtcttaacttgccaatcaggatgtcagttacccatgtccatacctttttctagcaagtaggatgtcaattcccaaggagatcttggggacttaaactttacttaacccctactcaaaatggaagccttatttttaaaaagcttcttatattggtgcaggtgtttctcttatgttggcatccatccaaggggctgcttataaaagcaaaatccatgaaagtttatacataggtgtaggaattgctgctgggttgttggtttgggtccaagctattgtggctaactatacaaagcagttctgctgacttctgttgcgattggtttccaagaacaccaaagcacagaagttaacagaatctgcaatcgactagagcatgagaaagtttccttatggcattagtaacttcacaaaatggcagactagaaagtaacagttacctaggccttaacaaaattaattggcaccgaagattctcactttattttcctcatttcttcaggttgacgagtccaacctatgttttgagagtggcagtttagtccttcacctgtggaggggaacagcactgagtttggaagttaggacaaggggacccatagctgttgctgagatcttcaaacagctttaaagcttagcatgtgtgacacccatttgcctccccatggctttgtgctgcccttcctcacctgcctagggaaggggaaggggttctggcatggaactcttggagggaaagttttgactctgcttgtagggaaggctgttaggacaggtgaaggagagacctctgacccagaggcaccggatctttgaaaactcatgtaccggatctttgaaaactcatgtaatctccacggacttaaaagataatatgtgtgagagaaaagctcttcctaaagagttgttttttgtttttgtttgatatagggctttttttttctttttctcttcctcttcctccttctttattcttccttctttcttctttcctccgcctcctcttcctcctcctcctccttctcttcttcttcttcctcctcctcatcactgtagcccaggctgacccttgaactcaaacattctcctgtctcagtcccctaagtgctggtataacaggtatgagctctatctggcttgtctttaatgttttctaaagtaaagtttggcccttttgtagggaaggagtcagagttagagtcaactagtatggtttgctatatgttcctataaacactaattgtagctaaattgaaagttgaccttcattttccttctagtctaatggatgggaccccaatgacatgtttcgatataatgaagagaattatggtgtggtgtccacatatgatagcagtttatcttcatatacgtaagttcacagagttcccttttatacattttagtgcatttgtctttgattttcatcagctcaaaattactttgctaactaattatttcttttaattttaaattaaacaacaatatcacattgtttctataaactattcagactgacagactcacattaaaacagggaataagtgttgtttccttacctatccttagttttctgatcaaaatacatttttttctgtttttaatttgtaaataaaaaatgttaaattgacagggataagggtgaaccagttcttgaaatagtaagtgttcttctttataacataaccacaaaaaatagtattttgatttatgatagaaagcccccaactttgtcagtgtctttgaccccatactatagagtctgttaatagctgaggattaaaatgatcatgcatgtcttcttgtctttgtgtttagggttcctttagaaagggacaactcagaagaatttcttaaacgggaggcaagggcaaaccagttagcagaagaaattgaatccagtgctcagtacaaagctcgtgtcgcccttgagaatgatgaccggagtgaggaagaaaaatacacagcagtccagagaaactgcagtgaccgggaggggcatggccccaacactaggtattcaacatgattcaaagtctgtgtgagaggatgtagtctgactgtttcctttttttttttttttggtgaagctaaggagaaagtgctccttgatttggcttaatattctgttggagagctgggcagtagtggtgcacgcctttaatcctaacacttgggaggctgagttcaatgccagcctcacccccccccccccacacacacaaatttctgttaaaagattacggtcattttaatagtatcatttgggattagctctctggagtcaagtgacccatgtgaggagccagcccagactctgaggagctccttaaaataaggttggaggaggcagaggcaggcggatttctgagttcaaggccagcctggtctacagagtgagttcaaggacagccagggctatacagagaaaccctgtctcgaaaaaaacaaaacaaaaaaaaaaaaagaaaaagaaaaaaacaaatccaaggttggacagtgtggcttgcatgatgaaggtcttttcagagcagtagaatatcagaatggggaagaactagcattgcctgtgtagttcaagagagcctcaccaggaggtgacaagtttggtatgataatggatcctgtctggccctgggagtgtggggggttatttcacctctgtagaacatggtcctgtaagagaatctggcctcttccggtaaagactaagatcccaggatattcttcatgctatgctggcaggtggcagaagcatttgtgtgccattcttgaagagtagtctgtagggaacagtgaggaacaaagacactgagtcagagagcctgtcagtgtgacagcattatagcagaaagtggcaagctgccagacagactcatggcgcgaaagctggaaaaactagagagaggcgaaccttacttgaaaattcagctgttcatgatgcttgctctgagaacacatgagctaaaggcagtgtggggtgacttagaaaggagaaacgatggcgcagtggttgcttgtatttgcagggctatgggtctgtcatttactcaaaaaagtagaatgcctgctgcattgtgggtaaagctgattctaccggcaaggttccatagctgagtcttccgatttccctaatcatcactgtgtcaggatgaaagtgagcacacctttttaaagtgctggtggccgagacctgcagggctgtgtggccgtgtggtgagactgaggcttgtgtgagagacagtcttctatttagtgtacctttcagtgtgttttgttagcccagtactttttaggataaatgatttagaggatctctcatttccagggacaataaatatattcctcctggacaaagaaacagagaagtcctatcctggggaagtgggagacagagctcaccacggatgggccagcctgggccaggctccatgccgtcaagagctgcttctcacacttcagatttcaacccgaacgctggctcagaccaaagagtagttaatggaggcaagtattttgaccttatttgttaatgttttgatgaaatacagaactatctaaacatttaagagttggcatacctgataggaatgtgcatgccatttgtggattttttggagtagtttttattatctataatgtagtttgttttgtggctactagacagtataacactgtatctgtcattggcgcgtatagggtgacagcatgtcctgagctgtttcatcctctcatactcatttggcctaagacactccacagacagacagaatggaaacttgaaagacactgggtgatgaggcagtgggcatagttcatgagaaagaccctgagttagtgcagattccttcattaactgtgatgattccattctgcgtacttcagggaagaacttagggtgatttgtaatgtcagaaatgagcaagagtcaggtgacgaaagtcatcagagaactgcttacaaacacagcacagacaacctttgatttctgagagtaggtattagagtgtagcagcttggagctttaagtaggaaggaaggcattttgtgtagtgtccccagtcagttgaatacagattataggtgagtatagaaatctggaagatgggacagcagaagcgaatcatttagaaatatggtagctgacagggaaaaagagtggacagcagattcagggttgaagtttagagcaccgcaagctgggtgaggcaagtgcctcatgctgagcagtcaggctccataggcagtctgagccagcctgggttgtgtgagacctcgtgtaaagaaagggagtgaagtatgtcagccatgtaagagcagaattcctctccaaggaaacactcttgagtagttctcctgcttagtaacgatggtgacatttctttgctcactgagtgaagattttcagttgtttatgaatagaattttcaagttaaaaacgtgaagggggctgagaaatgtatgatactccagtgagtggagccccctagactactggagaggaaccctggactgttgactaattaccagggacattgctgaccggggcgggggggggggggggggttgtccgtgccctgatgtcaggtttgggccccgaggcttacaggcccctgtggtgtacaggcagtagatagacggggccattcattcctgtgcatcaggatgagattcttttaagcattcgagaaagcaatagaagtaagctctcatttctacctgaacaggatgtggctagtgacctttgaaactgatatttcagttcagatataggcctgaggaagctcaggtgagagtaggagaagagcctggacagtggcgttagaggctgctgtttgtgtttattgtttgggcctgccttcccgagcaggagtcagtgtgtttattgagggggttatctgtgtgtgggacattttcttcacaagtgatggctgacaacagctgaagaagtaggcatactaggcagagtcttaactgtcttcttcagtagtatgtgtagatcctaattaaccctttgggaacgtggattcatttcaacagacttaattttaaggaggttaaagtaaaatgtgaatttgtgtcagttaagttgtactaaaacttatcacaaatcaaatgactgtcctcaaagggttaaatgtacaagaaatcatttttgtcattttacttttttttctgtttactttttccctcatttttttctttagtttttatactttccttcatatcatttgttctgtcaggtgttccctggccatcgccttgcccatctccttcctctcgcccaccttctcgctaccagtcaggtcccaactctcttccacctcgggcagccacccctacacggccgccctccaggcccccctcgaggccctccagacccccgtctcacccctctgctcatggttctccagctcctgtctctactatgcctaaacgcatgtcttcagaaggtacaataccacgatttgttcatgtttttgtttgtctttgtttagctcctttgtgaggttatgattacaaaatagtttcctcttcattatttaataacctataatttttgtgttttaactttagtttattaaagctatttctaataaccttttgttcactagattaaaatttgataagtgctatgtataaagccctgcactgccctgcatatagagatgggctccgacctgtctggtaatagtactcctgtgcagcccaggaaggtctcggcctggacagtaacagtgatgaactgggcatttctgctcctggaggtgggtggggttatggaggctgccacattcagctccttggtaatggtgccagcgaaccttcctgttggccaaaggcaggagtactattttggggttcttagtgatccatgacatcccttgtagtggccttgtacgtctgccctcaggtaacagtagtatttagagtgcctgaggaattactaatttaattgaataattattttaaattagttttataatatcactgtagtggaaggctgctttaatctgtggggagacctttatttaaagtttatcaattacaaaacaggtatagatttgagccttttctgtctttttggctcatgccaaattgtgtgtatttgttgctgaattcaccattgttgcatgtcatatactttgattagtggcacatgcctataatcccagtgcccaggccacaggcaagaggattgctgaaagttgaggtcagtttgtctcatatataatgagttctaaggctgccaggactatatggtgagaccctatcccagccaccctgacaccctgcccgaaaaaggtagcattctgatttaatgcaggttgtaagttctaagtaacaatgtcctttgttcagagcatttgtgtttatattctaatcaccttgtcttggtaaaatcatagtgaatataactgatcaagaccattaaagttttctaaagaaggtgtttgggaataaactatattggctactgttaaatttttgcccacacagaagggaattcaaggcagaagaatacaatgttgcaaaggcagatgtcacagactttcaatgcgtgcctctctggtgactcctagcagcaacagggagctgttgcagacgtttgggtggaggtgcttattcctcgaatgtgtcagaaagcattgcattgtagtcagatgaaagggctgcttttggactgcccagaaagcgtgccatgggaaaagtgcaaaaagagaagtgactccacaaagatgacttcacacacacacacaccagcacatacaaacccagtccataccacacatatcctatagtaatgaatacattttaaacattaaataaggggaaattctaacatgagttagcatttcctcagagcacatgtctgactgtgttcccactgaggacacagcagagagcatgtgctagggtcctcatcatggcctctgagaggaggggtagttctggacttgtgtgctacaggctgtattcccagaagtccccatgggtggcagagacagtgatggtgactgtcgtcagctctcacagtgcagctttctgcagactttccttgttctgctcttacaagccaaccacgcacatgtgaggacggcttgggcaaaagatggagaggagatctgaggttgctgcttaaattcctttttcccctggggccctgaaggatgtagctcagagatagaacactagcctaacaagtatgggctcccaagtttggttcctatcaaaaaaaaaaaaaggtttttgtctcattttggaaatagtaaaatctgttagaaagataaactgataatatcccatcatgcatctgtgcttcctacgaaaacgttattataacttatttttaaatgcatgtagttaaacactgttgggccatgttgttatatttctcaccatcaagtttatttcctccttgctttaaaagtaaaaggaaataaggctggaaagatgggtcagctgtcagacagctttctgctttccagaggaactcaggtgcacaccctcacacagacacatacacacaatataattaaaagtaaaacaaattgtaaaaggggaaaataaaacatactgacataattgacataccacaaaagaattcttagtgtggccagaaatcaataacagtgtgattcatattctaaccaatgaaaacctgctaaggaaataagagttatatctgtatctagtaggaaaagcagtaaggtgggcaccgcaggactgatgtgatatcctgtggcatccttctgtcttcctcaggacccccaaggatgtctccaaaggcacagcgccaccctcggaatcacagagtctctgctgggagaggctccatgtctagtggcctagaatttgtatcccacaatcccccaagtgaagcagctgctcctccagtggcaaggaccagtcctgcagggggaacgtggtcctcagtggtcagtgggggtaggtatgacactgcagagctgaagatctgagattaaatacagattaaatgtggtggcataagcagctgttgccagttaccatggttatcaagacacgagttgaaggaactgtcccatccctctggaactctgaccctgtatacattttgtaaacctgcttaggtttcttactgctgcgggtttccatggcatccgtgtgtggagtaaacagatagtcgcttggtgctcctcaaggagtagtctgcgccaggagaggctcagcttttgttatgagtctctgaccgtcagcagctgcagccaaggtcctgcttggagcagatttggaattaggccagactgcttgggctgctgagttggtcttgttggtctgtagtgcttcctcccgggagtccagagctagagcttgtgcctgttcttttggccttaccaggaaaaggtctttaagcttaaaaaactagacagacagtgcgctcacccctgtaaggtgtgcagttgtagtgattgtcctgtaaatttctttgtgctattcttcttagccttttaaactgaagattaacaagaaaagaagcttgaggcgttttaaccctattataaataagcaaccttttataaatacaagaatagagacagttgagggatatagtgagaatcgtgttacggggttggtctttccatggaaagttagagttgtgaacagtttccttctataatgtttgattgaaccattttcattgacccctttagtctgaagcctgcaaataggtttttattggattaggaagcattaagcagttgtgtggaaggcgttcattctgtgttacgtaccccttgaggctaggtgcgtcctaatttccttcagcactggcggtggatctttatattgcaagtaaaagcacataggcttggcagcaagctaccatcaattttatttcgtgtattgatggaaggagaggagagaggcatggtagagatctctcaccttgaataaatgttacactttgaatgtcgtctttttttttaacttaagtatgctttggcttgccattgaacttgactcaaatacttaaaagttcactcatttaattaggagggaaaccacggtggtacttagagattgttgacttctgccataagatacttctgagaatttaatgagtatctttttgcttctagttccaaggttatctcccaaaactcacagacccaggtctcccaggcagagcagcattggaaactctcccagcgggcctgtgcttgcttctccccaagctggcatcatccctgcagaagccgtttccatgcctgttcccgccgcatctccgactcctgccagccctgcatccaacagagcactgaccccatctattgagggtatgcaaccaacacctttcctgttgtaatccatgcatggccaagagttgtgttctcaatacacagtgatgcttttgtggatgcaagattctgtctctggtagaactattccatgtatattaactaatgtctggcatcagaggctctggcctgtcctagtatgacgtgcctgttgtatgagcatggttattgaattttgatgtgatgactaacagggaataaactcagtttaatgaaacccattttcccctaagccagaaggttagaagtgtttggtatttactcacactattgtcctaatttctgtctaatactagttttttgcttttttaaacttgttttctagcaaaagattccaggcttcaagatcagaggcagaactctcctgcagggagtaaagaaaatgttaaagcaagtgaaacatcacctagcttttcaaaagctgacaacaaaggttagagtttgttaaagtttttttttttttaaaagatttatttgtttattatatgtaagtacactgtagctgtcttcagacactccagaagagggagtcagatctcgttacagatggttgtgagccaccatgtggttgctgggatttggactctggaccttcggatgagcagtcggtgctcttacccactgagccatctctccagcccttgttaaagtttttagacacaattatagaaatagcaaagtgtttagccagacagatacttgcattgctagtctttacgtgatagatctgtcttcatttcttatttatctcttcccctcagccccagtgctgagattaaagatgttatagaggtgcaccaacactcctggcctctgttctcatttctaacattccatatttaacaattttaagtttaaaggtaaaagtaaattggaatctaacaggttttggttttacaggtatgtcaccagttgtttctgaacacagaaaacagattgatgacttaaagaagtttaagaatgattttagggtaagttctttatcgactggttagaattaaaaaagaaatagcaaacataatgatttttatggccagttcacaaaagtacactattaaaacacacgcacacacatacgctcttgtcttaagtcatttgtacagcaagctaaacttcttttcagttgtttttcatgcatatgagtgtttgcattatgtatttgggctatgggtctgcctggtgtcctttgaaaccagcagagggcataggatttcctggatctgaagttgcagatcgtcatgagctaccatgtaggttctgggaactgagctcaggtcctctggaaaagcagccagtgctcttaaccactaatgtctttctccagcccacagggtttgagagagtgtgtgtgtatgtgtgtgtgtgtgtctctagtcttgtttgtctgtgagtaggtatgtacatagtacttcaagtgccctctgagggcagaagaaattgttatcatttggaaatggggttaaggcaatggtgaaccgcctgatgtgggtgttagaaattgaatttagattctctggaagagcaataaatactgttaaccactgagccatctcctcagccccttgacaattactcttaaagatgtctttgtctgttctaagtacaataacttaagatcacaaggtttttgtttgttttttggtgtgtgtatgtgtgtgtgtgtgtgtaacaagactgcaggtaacaaaggatatgatcttattttggaaactgacacactagagctcactgctcagtagttctaaagttgaagactgaccacgtcagtgcttgtagtgtgtgcagtagaagatgaaatgtgtcctgagttagctcaccttccaatctgtgagtaaattcattcacttgttcattcatcctttttggtgttgaggattgaagccagggctaaattcccttggtgggcgtattcctgtaacctcacttcatatcctaagacaagcttccaagccctccttggttactctgtcattcttccacacaaaatttacaaggagctttagatactacgttagcttatataacttttaaaaatgacttctttgtttttattttatgtgcattgatgttttccctgtaagtatgtaagtatgtaagtatgtatgtatgtatgtatgtatgtatgtgtgaggatgccaaatcctcagaattgttgttataagcatacgtgagcagccatgtggatgctagaaattgattctggatcctatggaagagtcaccagtgctctttaccactgaaccatctctccttccccccaaattacgtttagaccaataactttttatcctttgaattctatactgttggggttttttgttgttgttttaactatgagtttccttttgttttgttttgttttgttttgtttttttcatttttttaagatattttatttacatttcaaatattattctcattttcccctttcctagtttcccctccgaaaatcccctatccctttccccctctccctgctgctacccgacccacccactggcattcccctatacctgggcatagaacctttacaagaccaagggcctctccttccatggatgaccgactaggccatcctctgcctgttggttttcttttttttgtttgttttttgtttttttgagacagggtttctctgtatagccctggctgtcctggaactcactctgtagaccaggctggccttgaactcagaaatccgcctgcctctgcctcccgaatactgggattaaaggcgtgcgccaccatgcccggcaggttttttttttttaagatttatttattttaatgtatgtgagtacactgtatctgtcttcagacagactgaaagagggcatcagatcccattacagatggttgtgagccaccatgtggttgctgggaattgaactcaggacctctggaagtgctcttaatcactgagtcatctctctagctccctatactgttggtttttatttttattattttttggttttggttttgttttgttttttgttttccgagacagggtttctctgtatagccttggctgtcctggaactcactttgtagaccaggctggcctcgaactcagaaatccgcctgcctctgcctcctgagtgctgggattaaaggcatgcgccaccatgcctggctattttaaattatatgtatctgtgtgttagaataagcacatgaatggaggcactcccagggactggtggtgtgatatttttctagagctcgttagagctgcctagtctggtgctgggagccataatctactccgttgggagagaagagttcatgatcttaaccactgagtgagccacccatcctccccaccaagttacattgaaatacctgttcagggagtcttgaaatttagctattatttagcatttatatacctatatatacatgcttttgtatctctgtattaaatcttaaaactcacaaatgagagaaaacatgtaataattgtcttttctgaatttgcttaatctgatactctgatgttatatttattttccaattgttttttggatgactaaagctgtgaatacccatgtcacatccttctcagtcagtcctctgttaatagacacctggcttggttctgtaagtagctattgtgaatagccctgcaagtgatgttgataaggtacactatctgtgacacggctttggagtcctttgagagttaatcgctgggtaaaatggtaggtctatttttatttttttgggtttggggtttttttcatgattgactttacagattttatttatatgttcatatgtgtgtgtttgcacatgtgcaggtgctctggagctgaagttgcagacagttggtaagcctgagtccatgtgggtgctgggaattgaacctggaagagtggccagtgctcttaactactgagccatctttcctgccaagcgctttttagtccatagagactggacagctttacattcccaccagcattgttttctggtgggagcaaaaagcgttctttttactccacatcctcactaccatttgctgtttgtttctaatgactgccattgtgactgagttaggagagaaactgagtgtgtgtgtatgtgtatatatcttgtctaaaataagcgcatctctgtgtatgtgtgtgtgtttaagtattcatgaatgaggatacctggcggggtcacaggctttggatctccctagaattgtgtgattgtgtgctcagtggctggtgttgtgtgttgcatggtgcaggtgctggaaactgagcttgcatcgtcttccagaggacttcatactcataaccacttcaaactccagtttcctcaatggagtttcaactcaaatttctctcgtgcctagtgatcttgaacatttgtcatggattcattgactattgatgtgagtgttttgagaacagtcttgtgtgtttcattcgcctatgtaaatgattttgttatttgagatacatacaagtaagtacacacatgcacacacacatatgttgggagtactcagctgtcctttgttacacatcatgctctgggttattcttgcttccttacaaggatgaataatgccaacagaaacactgaggcttggtatcatgtaaacatgtgtggtcacttctttaggattggaaagtctgtgtcatgtggtagtgtgttgagtctttgcaggaactaccagtcattgtaccatggtagctgcatggtctccattgaacactccctccaccatcagctagcaagttctttttattttcattttgggtttgtttttttttttttgaaacagagacttgcattgtaactgcagctacttcgtgtcacaggcacacaccacctggccatgtgtggggacagccattcccctgaaggtgctggggttcatggtcttagttgtggtccatattggttttcatatgcagagctctcgtggccagtgatgtttattgccgtcctttgtgttcattgtttgcttgttttgttgttgtttagattgctgttttttcatatcctcaactgtaaaactttagggtgggcactggtgagctagctctgtgggttatggcagcctaagttgaactccctagactcacatggtgggtgaaggaactgacttccagaagttgccttccagcttcccataacccccttcctgtatgtgagataattaatctttgttcttttttttcttttttttaattttttttttttaaggttcctctgtgtatctctggctgtcctggaacttatctgtagaccaggctggccttaaacttgcagatactcctgcctctgcctcttgagtgctgggattaaaggcatatgtgccatcaccacagctcagctgctaagtaatctcttttaaactgggtttttaattaatgaattgcagtaggctatgttcgtgttccttctttttcttttcagacagtgtctctgtgtagcccatgctgaccccagcctttaggtctctctctctctctcactcggctggggttcttatgtactgtagatcccagtctggtgccgtttgtctaacacttctgggatattaggtttggcacccagagtgacacagagctccagccttggtcctgtatccgtcaaaggatctgggtgtgtgaattgcttttctcccatttcatgggctgtcttttcactttctgtcccttgagacacagctttatttaatgacatcaaggttttttcctggtatcagttgccccttagcttgagagtttggtgtctggtattgcaaatgactgtttaatctcctgaagatggcagcacttaacctgctttcttaggacttcctgggagctctcatctgtgtggttcaccttactgagcgaggctcgcactcagttacactggtgtctatcaggaatctccccgtcatctgtcgaaaatgggactgtattcacctccactctggctccaagttgcttgcagatagggaagtttggtattagctctccatttcttcccatgtccatgtgttgtagttgatacctttttgaaagactattctttctagacttaatatcgcatgcctttaatcccggcacttgggagacagaggcagaggcaagcagatctctgagttcaagaccagcctggtctatgtagtgagttttaggacagccagagctatgcagagaaaccctgtcttaaaccaccctagcccccaggctgctctttatttattgtttacttaatattccaaccactgatactagtttgcaactctctttgtttggccctaccttactaatgaattctttctctgtcctgatacttattattttttccagccttgatttgggcttaggttgtcgtcactcttctgatatcttcaggtacagcattgttacttattgagatcttatcaaattctttttttttataaatttaactgaatttaattattttaatttttgtttaattaattaacaattgggcattacatttcttctttaaattaaagttcaggtagcactggcatcaagagtcggtctctaggagtggatcatacttattttgttgagctcctgggcagtgagtaaatgctaggaaacaaagcaggggttccagggcagggttgacactgagtgcttcctgtgtttcagacacttttctctgcttgtgtcctagcccctcctgtcacagtgatgctctgaagcaggttctgtcactgcctgcagtgtgtgcatcaggaaactgaggcagtgaggttaaatcatctgcccagggccccatggataggaaagggcaagccttggctctctggggtactgcagtgttcagccatcctcgtgacatggaggtttggttttgaactgcccttttcttgagagcagtctgcactctgcagtatagctgagtattggaaatttgatacagaggatttctctgtctgatctgttaatgcttagtaactgagattatgcagatagagatttttctttcacagtatattgcctagtgtcccaagcacagtagggactggtagaacctacttgggttgcaggtggcagcatataactgtgggccctgcccttgctagtgtctgtggtccacgcttgttactgggcatgacttcacattgccgaggtgtggtgactggtcctgacagttgcttttctctgagttacaaaaaggcatgggtttgggtggggtttagctctgtgtgtttactgtggactagttgagtccagtcccaccaaacacacatatcccttccctacagaatttttaaaaaatatttgtttatttatcaaatgtaagtacactgtagctgtcttcagacactccagaagacaacatcagatttcgttaaggatggtcgtgatccaccatgtggttgctgggatttgaactcaggacctttagaagagcagtcagcactcttaaccgctgagccatctcatcagcccctccctacagaattttaattgcactgttttctccatgggtttgccataaaagtacctacatcaaaatttaggccattgtgtggattaaacaaataacacaaacagtttggatctataggtaattttgccattataagaatagaaatgagctgggcatggtggtgcatgcctttgatcccagacagaggcaggtggatttctgagttcgaggtcagcctgtctacaaactaagttccaggacagccagggctatacagaaaaccctgtctcaaaaaaacaaggaaggaaggaaggaaggaaggaaggaaggaaggaaggaaggaaggaaggaaggaaggaaggaaggaaagagagagagaaagaaagaaagaagtggaaaatgcttttttaaaattttattcctagcatgtttttgtatgatgtgggtgtgtgcccgtgagcttacatcttcaggcttgagcaatctcttcagctctttgcattttatgtagattcatttttatttcatgtgtccggtgttttgcctgaatgtatgtgtattagtcagggttctctagagtcacagtacttatggatagtctctatatagtaaaggaatttattgatgacttacagtctgcagcccaattcccaacaatggttcagtagtagctgtgaatggtagtctaaggatctagcagttgctcagtcccacatggcaagcaggtgaaggagcaagagcgagactcccttcttccaatgtccttatatggtctctagcagaaggtgtagcccagataaaaggtgtgtcccaccacacctttaatcccagatgaccttgaactacctgtcttaatcttctggaatccatagccactatgcctcaagatctccataacaactaagatccaggtcagaaacttctatctcccagcctccagagaagggtcactggtgagccttccaattctggattgtagttcattccaaatatagtcaagttgacaaccaggaatagccactacagtatgtatgtaagtgcactacatttatgcctgtacccagagcccaaagaggacaccagatcccttgaaactaaaattttagagattgtgaccatgcaagtgctgggaactgaacctgggtcctctgcaaaaacagaacgtgcaaatgtctactgagccatcactgcagccctaatttttaagtttaggaagttgtcgtatggtcattgtatattgtcctaggtttcataagcacattcatataaatgtcgatgatacactctgagcacattccatctcctgttctctccctttcccctttctcatgctgttgggattttaaaattagtttttgtttagattagatgttgatctcttcaggagctattccttcatggaggctgctttttctttctgcgccacatggtagcttcccaccttcctgtgtctaggcagtcggaactgttgcatcttgttcactgtcagccactgcctctgcctctccaattctgaaattgcaggcatgagccaccatcagtctaatagttaattcttttttgttatgccttgggcgacaggggcgggaggttgagacatggtctctctacatagccctgcttgctctctgggaactcactatgtaaaaactaaggtggtctctaactctcagacgtgcctctgtttcatgagtgctgaacataaaggtttgtgccaccatgttcagcctggctaataactagtcttggatactggttcctaaaattgtgttgtgtatcattctgttatttgtgcctgaaaaatgcatattctgagccaggcatggtggcctttagttccttccagcattcaggaggcggagaaaggcagatcacagtgagttcaggaccagcctggtctacatagagagttccagtacagctacactcaggttacacacagagaccctgttctgtcttaacaaacagacaagtccaccaaaaaggccatattctgaaaataatgaaaacagaaaacctattcaaagtattttcattgccatcacatactacttggcttatacttaatgggaaattatttcttatatttagttacagccaagctctacatctgaatctatggatcaactactaagcaaaaatagagaaggagaaaagtcacgagatttgattaaagataaaacggaagcaagtgctaaggatagtttcattgacagcagcagcagcagcagcaactgtaccagtggcagcagcaagaccaacagccctagcatctccccttccatgcttagtaatgcagagcacaagagggggcctgaggtcacatcccaaggggtgcagacttccagcccagcctgcaaacaagagaaggatgacagagaagagaagaaagacacaacagagtgagtaacctgggtctcagcacatgctctctccgagtaacctgctttaaggggttttctgttccatagtaactgttacatagagctggggttagaaatctgagctgaatctagtcattgggttcttagcattcttcccattctgacttgtttgcagctgtgggggtaagaaaagctacttctatggagataagagtttaaatttgaatgccctgtctcggggttctggttctgcctaaccctgcagtctgcaaggactatttgctgccccagttgaggaaatgtttagttgataagcacatctttataaaccttatttctgtgcatctggcatttaaaagcacttaaatagataaactagtgggttagcctcagacataatcaccactccctttttgtgttctggataacaaaaaaaaaaactaattgtacttgaactatttattcttgatttgcagaaaaataattttactagagcaatggtaaacccaaggcatgtgattaaaacattttgacacatttaaaattgctgcgtatgctacaggtggtggaagggctttgttaacacagcacttgggcatctgaggctgttgatcctaagtttgaggccagcctggatccattgcaaggcctaatctttaaaaaaaaaaaaaaattagaactactgtgaactcattaagcattggtgttttatctgcctgtctgtctgtacaccacacacttgctgtaccttcagaggccaaaagagggtgtcagatgcctgaaactgtagttatagatagatggattaataaacccagtcctatggagtggcagctgtttagctgtcacttaacctcattaggcacccttttaaggggctgcgacttaccagttgaaatattcggtgattgcttttgagggatgtggtttctttggttttcatttgttctcgtatggttagtttcttcatcagattaagcagcagaggtgtgtgtttcagtaaaggagactgagacatggagagttactcacatctgctctatgtcagcaatcctctgaaagctgacgagtgtggcgcagttgagaaagccgcagccccatgatgtttcactggcctgcttgttgttttctttctttcttaggcaggttaggaaatcgacattgaatcccaatgcaaaggagttcaaccctcgttctttctctcaggtaagcgctagccttttacagctggtctgaagagacgtcactgcgctcttcatggctttctcctgagctcccatctgactgagcccagttgtaggaccctgagacagcctggcctccaggcttgtgggatgtccccaagcttgtcagtcctgctgtgccgctgtgagggacagctcagtgtaagctcacaacgactgaggctgtcaggccaaagaagcccactcactaactacacaagagagtccatttcctcgggaaggcataggagaacatgcctgtattcttagcaaacaggagaccggggcaaaagggtcatgattttgtggtcagtttgggccacatagtgagatccccaccccaaaacacaaaaatgaaaagaaaaaaatggatagaccttttctggttccttgtaaaattattacaatttttatatctttttttcattgataaatattttagcttgattagatatgatacagtgttgataccaaatctgtgtttatgccagccctctaacactgagaaatgtctgcagcctaagttttcccttttttaacccctggaaactgaatcaacttcttacatgctagcctctggatgttttgtttgcctgtttgtttgtttgtttgttttgctttgctttatctttagaataaaaaaatgctagttaaaaacaaataggcagaacaatcagggaagctgctttgaatattgttttcatagatgcttttcatcttgacagagttgatgttgaattctgtttttgttgttgttgttgttttgtagccaaagccttctactaccccaacgtcacctcggcctcaagcacaacccagcccatctatggtgggtcatcagcagccagctccagtgtacactcagcctgtgtgcttcgcacccaatatgatgtatcccgtcccagtgagcccgggcgtacaagtaagttccatgggttacaatttgctggacaccatagcttgcatttagcacaagtgtcctaggtgggcactaagtgggacacctgatagcaagtagtgttaagagtgggcagtgggtaggggtggtgtattgtatcattagcatgtggatctaagttttaggattaattttcagaaaaaaatgacttacatttactataggccagagttgttccttttagttgtgggtattaatttgaatagctcacagggaatcaagaaaacactggccagatcacattaaaagacagtgatagaatgaatatattgttaccatatgaatctaatgatgtgttttcaattccctcaagcctttatacccaatacctatgacgcccatgcctgtgaaccaagccaagacatatagagcaggtaaaggtgagaataaccctgcctgtgtttgcttgtgtctgcatgctgcatgagtttagcacctgtgtgcaatgaatagttggcttcagcttgaattcacagggtatgtgttcacttgtgatggccaacgtgagtattcaagtgatcacatagtaatcatgtgagagaaaaaaatataaataaactttgtatgtggtgtgtgggtatgtgcatgtgtgcactcagtatacatggaggttaaaagaacactgtaggagttcctcctggtgggttctaggatcaacctcaggtcatcagatttggccacaaatacctttacctgatgagccatcttgccaacccaataaatgtccatccccatcccagtcacctacacctttaccacccacactccaccctaccctacccccacacagagtctctctctctcttaccttgtagccttgactagcttggaacttactttatagaccagacttttgtggaattcctataaatcttcctgcccccccaagtgctggcactaaaggtgtgcccactacactcggccttctttttaaagatgcattttgaggctggagaattggatcattggttaagagtgcatgctgctcttgcagaggacccaggcttgatatccagctctcacgtggtgcctcacgacttatctgtaactaactccagtcccagggaatccagtgccctcttctggactccatgcataccaggcatacacacatgtatcaatatacatgcaggcaaaacactcatacacatataaatatctgtattttttttctatatatgaacattttgctagcgtgtttgtacttgtactgtatggacacacggaagccaaaaaaaggcagcagaacccctgggactggtatcacagacagctgtgagctgccttgtacatgctgggaacctgaactctgatcctctgtaagaacatcaagtgccttaactgataattctctctagcctctgtttggtattttttgccaaaagaaataaattttagaaatttcaagctgaagtctaaattgaaattttatcagaattttttttttgtttgtggtttatttggaattgttttcagccatagtctcactgtgtaaccctcactggtctggaactggctatgtagaccaggttaacctcaaacagcaatcctagagttaaaggcgtgcaccaccacactggccgctctttattgctgagaagatgctcctatagccaggctggcctcatcggacttgctgtgtagctaaggcctcagatcaggtgttggctgtgctggaactgagtctaggtggcagtgagctgctatgtgatgctgcggacattttccccaagtgctgtgtgccctgcgcacagctttactcagtgctcttgccctgcagctgttgtaatcccctcttcccagcgtatcaatcctgctgactccatccttgggcagtgctagttcaaaggagtcttaaatgccttccaaccctggaaagagggattactacataatagtcatttccaggacaaagttgggtaatttttttttttttgtaggtttgttgattagtttgtttttctggtattgatggtatttctgaggattgaacccagggcctcatgtatggtgattgaggtagaggaaggaagggagggagggagggagggagggagggagggagggagggagggagggagggagggagggagggagggagggagggagggagggagaagagagcatttaagttgcccagggtagccttacactctgcatgtgttccaagaagcctggagctcatgctgcagtcaccgagtgtagtgttctttcagtttgtgctgtgttgtgaatggtgtctgttctctcagcacagaggtgtcctgtcatctctcattgtgataactgttacagggaaactgaggccgaaggatcaggagttcaagtcagaagcacaaggtcatcttgagctacatgacaccctttacccacctcccctggacccccatctttgcgcaccacatgccttctgtagctacttcctacctgtgttgctgccatagataaccatggtgtagtcccaaattcagtcagggcactgtgatgtggtgaaacttctttcatccatctttgctttccagcaaagctttacttcattggacttgtggattgtcctgaagtagaatttgcatgagctggaatggaagtacagacatgctcctcagtgcctttttaaaggctgtctccaaagataagagtgtgttctatgctagggcattgtcaacgtacgggatttcatacttgtttccctagctggtattttacagttaacctagaggagattctctcccttcacagaggtgaaagtgaggataaacttgttctgtgtataaatggagaacctcgtgcatttccacaggttctgcgtcttaagctgctgtgacaggaatatcaactgtttgtgtcttctaaggctcaacttagaaagtcctgggaaagtagccatccctgcctatgtgattgctatatcctcatactgctgtgctgctactgaacaaatggaggtgctagacaaagacacgaagtcaagttctggcttctgagattctacaaaccatgtaaaagacacctcttcttgcatggcagtgaatctttgcataaaaccacatcaggttccccttgccataaatcacacctgcctttcatgcaccctgtgagaacacgcctgtaagggagctaacataatggggatcacacacacagagtcactgagcctactccggcctccagttctccttgtagctgtgtggatttgaacttatgtactcaggctctgagcttcatgcctacaccacactgcaagggctgaagtaagttgtctgatgctgggcaccattgccatctgtaactagtattcttttctacagtggggagaggctccttgcctgactgcgggtgcttattagcaatcactagatctagtagtaacaaccctttagttgattaggatgacaaaaaaaagtctctagatactgccacatggcctctgttgggcacaattgctcttaatagagagtcaatgacatgtgaaatggctgaaatatggttcatataaagtgtaatctaagatgaatatccattcagttttggagaaatccactatagccaagtaaatatgatgtcttttgtggagggcaggtatccatggctagtttgttcctcgttttgctttgtttgagtgtatttattttacagaggttatggatcccaaaacagcctcaaaagctgaggaccttgaacgtcattgtccttgtctcccacccaaatttccttttgttttctaacaatagcagttccattaaatgttcagccaacctttttaaatatttaaaataataagcacatagacaataatagttgctttagctctgtgactgcataaacaagccaggttacaggttctctgtgctgtaccctcatgaggcatgcagttcccagaggtagcatgacatcagaaacagacatgacagctacaatcgatgtgtgcttacgggaggaaatgcacgggtgatgcagaggacgtttgtgagtcttacacgggtcttctttcctctgtttcctgggtgttgagattaaagattcatctcttagctgacatcaggaaaagtaatttttataggaatatggaaaacacacagcaagcaatttttcctttcttaaatgcgctacttggtgtacttggtactgtatgatattggaaaacaggagagctgggcgtgggggggggggggctttcaagacagggtttctctgtgtagccctggctgtcctggaactcactttgtaaaccaggctggcctcgaactcagaaatccgcctgcctctgcctcccaagtgctaggattaaaggggtgcgccaccacacccagcttgtcatctgtttttcttcagaaaggaacctttcttgaccttcttaattaaacacgttatgatttggagtatcagacagtccctgtgttctcctcccattctaataccatctcttatgttaggaaaaccaggtgaagggaaagtcactggacagctaaactctgaaggatgagtcccttacctgaagcaagtggagcaatggctcaaagcagcaggagaggggtgggggctgggtaagacagctcagacaggcagaatcagagtttggtaacacagagcctttcagaccacacaggtgcatgaattataccgacaaggtttcattttcttttaatccgtttctttttctttcgtgcagtaccaaatatgccccaacagcgacaagaccaacatcatcaaagcaccatgatgcacccagcctccgcggcagggccacccatcgtagccaccccgcccgcttactccactcagtacgttgcctacagccctcagcagtttcccaatcagcctttggtccagcatgtgccgcattatcagtctcaggtaaggccagtggaacctaactcacttcactcctaagaagagcatcttacttcgattgtttatgggtttatgggttttttggctttgttttatggtatcagatagttaatggggccttttaaatatgtttctttttatgtgtatgcttagttttgcgtgttgtaaaccgtatgctgtgcttggtgtgtggtgcctgttggagagggcacttgctgttcttgcagacgacccgaggcttgttgtccccatacctatttcagaagatccttcggtctccatggacatctgcacacatacaaagcacaaatactaggcacatatacataaagaaaataaatcttaaattaaaaaggaaaaaattgaaaaggaagcatctgagaaatagaactgtgaaatgtttctgggaaccatgaagctaggagtcagaagtgactgggacagagcaatggctcagcagtgaggcgtgtttcctgctggtgatggggcccagcacccacatggccactcacaagtccttgtaactcctgttccaggggattcgtgtcttattcaggccccttgtagcactgcacatgtgcatgcatacactcaacatatacagtgaatctttttaaaaaagaaccttatctagacttcacatcgttcattttggcataatggatgttcttttgtctacatacagcatcctcatgtgtacagtcctgtcatacaaggtaatgccaggatgatggcaccaccagcacatgctcagcctggtttagtgtcttcttcagctgctcagttcggggctcacgagcagacgcacgccatgtatggtaggaaccattttattgtcttttctgtgtgtttgactatagttgctgtaaagctctgtaaagatattctgtttaagaatagcctgcgataggtatagtggactggatcctggctattagtggtagtggggagactaaagtacaggatcatttgagccaagaggtgagagacaacatagagactctaccttagaaaacaaggggggaaaaaaaagaaaaaaagaaaagaagaagaaaagaaaggaggagtaaaaataacctgactctttgtgagagtgagtgttgagagtcttaaaacgaaagcaaacttatgtctgacttaatggcaatttatctctactttgaatctacaattcctctgtcttgatatgtttctcctttaaaaaatgatgaaaagagctgggcgtgggggtgcacaccttcaacttgggggttcagggaaggggcaggcagatctttttgagtttgaggccagcctggtctccagagcaagtccaggacagtcagggatatactataagtaagaccctgtttcagagaaacaaacaagcaaacttgtgacgtgtaatgagagcatctttgggggaaagatgtacttaacgtccttcctgcccaagccttgcagggaagggtgtaccatacagtctttcttacttcccacatcttaccatgtagagtcgcctgttcatcttacagtatgcttgtgtgctttttacagtcaccttaaattactccgaactttaaatgacaacactataccaaacttagcacatattttttctagtgacatttgaattcttgggaaagttagataagtttgggttacttatcagttgagaagtgtacacaatgggactgagagtgtgagtgcatgcagccttgtacacactgtgtctcatcttagtctgaccccttaagtgaacatatgcagggcagtaaggtctgtgttgtggtgaacacttgatcattccgaatgctttaacattatgtaggctaactaagagaacattggccaccctgagtgtcctccctgtgtttgtgatcacatgtgtactgagcaggtgcacgcctgtcaccagtgatacatgcctgctgtcaaacacttgcctatgtgaaatcctgaggtctgcttgttccacattttgatcatggtcattgccatcgtcatcagtgtgtactgggcatttgaaataaagagtagggcaagtgctgtactttagaaaacattttcttttaagcagtcaatccattggcctggcatgggagagtgcctataaatctcagtcctgtgaacatcataaaaaaggaggatcaggtattgagacagtctgggtggcatagcaagaccctgtccagatcaaagtcctcctgtaatttgatttataaagagtggcctggtttacattttcattgatttataaagagtggcctggtttacattttcatcatgatgagtctgtccttcagtgtgtgatcagttgtactccctgggattagatcagacctcacatggaaaggccctggcggtgtctctcgctgaccttgctgtaccttgtatgcattcccacagtcttccagcttggtaaaaggcttggttgaagtgcttatgtttggacactgctgctgtgtgattcagaagtggaagagacatttctttcctcctggttttctggcccccactgggttaagaggaaactctggagctgaagtttgtagtgctttttggaacagcactgtggctgccagtgcagtgctctgcagtgctgccttcctggttttgtcatgcagtacctatataaaatgggcactaggaaatgaactgtttggggattttcccatagtcaaaaatcattttttaatacgttgacacagtttgtcagtaccttggctgctccatagatcagttatgccaaacatacatacatataagacatatatgtatgtatacacacacagtttgaaaactgttgtatggaaatactaattttgtgttcatttcctttgaaaagtatgtattttctataacccaagaaataagacacttgaagctgttgatttacaatgcagtggtgctttgtcctcagttctgctgctgatctgcctctctgttgttactggttctctcaaggcaaaggccctccttgtgttgaggaggactacatggtgtcaccttgtactctttgtgaggaaggacttctattagcagactccccttgttgcctctgccctgcaaagtgatcactgtagcgtctgtcctgctcgtctgagttgctattccagcagtggttccattttcagactcagtggtagagctgtctgactgacaagtcagccagaagaactgaatgtgctctcactagccccactcccactcctccaaaaaaagacagggtctctgtatagccctggctggccttgaacttgctatataggccaggctggccttgaattcagagatccacctgcttctgcctcccaagtgctaggattagaggtgtgaacaaccataccagctaaatattttttaacatgtatagtttaatgcagataagtttttttttaagtacaataagcataaagaaataggatttcctgtaatccttctgcctgacatctgcttgttaatattcatatattctcccagagctttaattatacccttttagacagacacattttagtatgaatagtgtgatacttaaatttatatttttatgtcatgaaccttgggatttactacccaagggcctgtggtgacatttgtgttgctgtgaaaaattatgtatcatgagtttgcttaaatactgttttatctgggtaccttgcttatgacaaggctgtttgatggatgccatgttggtctttttatcagttaacggttctctatcagataataatcttattctttttatttaagcaagtcagcgtgtggacttgctacctcagtacagaaggaaagccttccggttcagtcctgttcgcttgtaaatctctgcagcaatgcttatttacagtgctggtcacccctgaccaaacgaactgtgcgtgaatacaaaagatcactgaatgtgtcggcctttccagagcatcaggaatgcacacctcaagaggctaatttacctttctatttcccacattcagcatgtcccaaattaccatacaacaaggagacaagcccttctttctactttgccagtgagttggggtttttatactaattttaactacacagtaaacactatttaagggatacgtgttaaagtagctaatttgttggagaacattttctttctaccagacagaattattcaaagtatgcatgtctgggctggggatgtggctcagtggtggagtaacttgctctagcatgcaccaggccctgaattcattccctagtacaaaaaaaaaaaaggtaaagttaagatttgtggctctgtttgtataattcagagttttgacaaaagaactatgtcttatgtcatagcttaagaaaattggaatcaaagactgagttctttcaggaagccattttgaatacaacgcaagagtgtagctgtatgggaaataccttgttttcctcattctgggagactttttctgaactgttggtagtattttatactaagccacaagcttgtgcaaagcttaaagtactagtgagtagtgttctgggctgcagagaagtgacctcactgagattcctgtagcacaggtgaagtagttagagacacagttctgtcttagcgctatttgcatgactagaggcttaaagtgtgaaggcctctggaagtgtcctgaagcctgtaacaatgtgtggtctaggctaccttcttccctaagggagcttaggagatggagttttcctgctttaccgggaagcagatcatgggctttgataggctaattactaagagaccttttagaaatgtgtgtgctgatgtcactttgtatgaaacatgagtcctaacaaacaataagatttaatgttattaaatttgggactaaattttgggctattttggaatgactactcaatcatcagggttttggaaacatttttaccttaaattttcattaattagttgctactttttggtgcctgggataagttaacaaatagagccctttcatgtgctcttggaaaagtagtcttattgagaatgtccctgaggatttcaggagcacagtttgaggtctctagtgcagttatccttaccatcctgctggctgttacagtcatagtcatttccttggcagatggctgaaaggtcttgaatcatccctaagcactaacagcctgagcacagcttcctcatgaaaggagtcagcactaaagccagcagctgttgttggggattttagtcaccgcagtgttctgccctagtccactgcggttttgcctcgccttagtgtgatgtcttttatcccctctgccccctaaaatctccttgagttatatcccctttttggaattattgaagttggaggatcctgttctgcttattttcagtgtttccagtgctagtgatcacgaatgacttgtacatccaaggtcaaaatgatagaaactatttacagagtgaacatctggtcctgaggactgaattcgcttttgttaacctcatgacttaggggactgtttctggaatctagttaatgttaaaaaaagagttgacatatttagaattttaaatacttaagtcaagaagcgctttgtcctctcacccaactctcgggagactgtttgagccttgtaagtagtagtttagctgctgaataacgatttcagaacatctgaggggattcatggtctttctagtctcccaagtagttaaattaaccagcagaggaaaagccattcaatcctgcccttggctgagagccttgcttgccactctccaaagactgctcttggttaccttccccagtcttttctgttctgttgtttctctaatttccctctccctgattttttttttaaagatttatttatttattatatgtaagtacgctgtagctgtcttcagacacaccagaagagggcatcaaatctcattacagatggttgtgagccaccatgtggttgctgggatgtgaactcaggaccttcggaagagcagtcagtgctcttaactgctgagccatctcaccagcccctctcccgctttttattgcccttagttttgctgttgctggaattacagtgttggttcccatttcccgatttctcacattatagtcaaggggaaagggttacataaggtacttgttttttaggcctctttgagccagaaagtgagcagatggaaacatttctctggatgctcctgaaagtgttttgttaaaatgcatactagggcctggagagacagctcagcagttcagagcactggttactcttgcagaggattcaggtttggttgccagcacccatgtgtaactatatctgtaactgtagttccatgggatccagtattctgtgctggtctctgtgagcactgcacatgtgtgtgttgcactggcaaaacactcatatgcatataataaaaatcaatctttttaaaagatgtctaatagggccaggaaggtggctcaatgataagaacacacttcctgtgagagcctagcctttaaatgcccttttatataaaaataaaattttaaactggatgtggtagtgaaagtgtggccctgccatgtgaagagctaaggtgggagggtcagctgctgtcaagcttgagggttgcgtgggttccaccaagccctgcctctagttattcaatcttaagaacatggctcagtgtagctcagtggtagactgtgtacttaggatgcaggaggctctggctccaatcttaagcataaactatgagtaataaagctgtttgattttgatcctggctgggtatggtaccccggtgcggatccacttggaagatggagttggggagggtgggttccaggccaacttaagctataaaagaagaaaaaccaaacaaagaagggaaaggatcttttaattctctttccaagtgacagtatcaagactttcccattttcccagttgacagatgtaattctgagacttgatggacatagttactatgactgcggtggccgtgtttccactgcattttgctagagcttccttcagagttacttgttttcccacctgttccaggactggaactcagggccttctgtgcaaagctttgcactgagctgtgtctccaagccttttatttatgttttcttaaacttttttttagtttgaatacttgagctcctatatcctctatctttatgaaatctcataaataaacatttagggttgctttccctcagttggacttaacattgctataaaggaaacacttactaaacactggatggggctttaatgtttgtggttttagaatgagacatgtggaccagttatcaccatctagcagcctagtctatagttgcagacctgtagttgttgtctctaaaaacaaaagtcgttcttaagtgcttttcacagtcagggaattagaggatggactggtctccctgactcactgaaaacattgtgacgttaggctgccgtagttttctctttacacatctgttgactaatacagtcagggaaactgtaccaggttctaaacacagttgccttcatcagtctcacctggatactgagggacactggtgagtgagggctgtggccttggcaggtggacacacaggccatgccagctccgaccattggaaacagaatgccctgccctgcactgtgacatctcatgaatgtttcctgtccctaacccgggacaccactcagctggcacacagccagagtcctccatgtgtgctattcttggaagttttggtgcttctctgtccacatcatagctgtgtttgctgacatattatctaaagtgcatgaaactgtgacacatctgtgtgtgggcttggctgtggagggctaagggttcagtgctgagagagagatagagatagcttgagagcagtccattttctgcttgatgtttagtttgtgtcgtgagctgtccagagtcacaggagcagcgaccttcctggagttctcattcccaggctgctgctctgtaaaggcacaggttcacttaatacttgatctccttggttggtaccaaggtggggcccctcctagatgcataaagagcagcctcagggtcattgtcgtagagtcacattttaagagtgttaagaaaaactgttcctttggtatttttctagctaacaggatagagtggtcagtttcatctccaggctttttgtcaggagctgcatcattcagtagggaacagaatagtgagtgggtactgtgattgactgagatggcagtgacgttgtccttgcgcccagcctcactggtgtgtgtacagggaccgttccagagcagtagcatagcaatgtaatcagatgctgaccttgagagagaggctgcacctgtcttcatgtgggccagtgtgattggtggagttgtttctacctaagcctccgagaggaacagggattgtctgagtaaatttaaactaagtcggtgtgagtgtagtgtgtagagctatgtagcacacgggtcacgtgctggccttggtagacagagcccttccgtctgtttccagactgtgcttgctctcatggctgtagagcgtggcagtgattagagtgcagtgaattaggcatagtgggctggcagtggtcgtcagaattgtacacattcctttttctttttcatctcagtttccaccggctccctcgctcagcagtatgcacatcctaatgccgccctgcatccacatactccccatcctcagccttcggccactcccaccggacagcagcaaagccagcatggtggaagtcaccctgcacccagtcctgttcaggtaaggagaggcgtggcctgtgtgtgcctagctgtgagcagttgtgggtacgggggaggccattccctaaggttgaatgccagaaacataagtgttaaaaagctgcttgatcttgaagttcttagttaagtctaaagccctctggaatttcattcctgaaggcaagccgcaatgctgccttcctgttggcttcctcagtcccatgtgctaatggggcagtgaggccttgtttggacctgtaactgcccggctgaggcttgggcttgagtccccggaaaaggccatctgcatgagagatgctttgtgctctcctttctgcgcctcccggggtgggggtggggtggtggcctgtcgccttcactctatgaagtttcccattgctttacagcaccatcagcaccaggctgcccaggctcttcatctggccagtccacagcagcagtcggccatttatcatgcggggctggcaccaacaccaccttccatgacacctgcctctaatacacagtctccacagagcagtttcccagcagcacaacagacagtcttcaccatccacccttctcatgttcagccggcatacaccaccccaccccacatggcccacgtacctcaggtaaaccagcactagccaagtttttgtgaaggccacatagggagtggtggtcagataagcagcaaggagccagttgctccccaagccaggaaaccctgtgtgtggtgccatctgcctgtctgcccctttcctgcttagcacgtgatcttctggctctcagtgaatgttgctatggttttgtggtggatcagtgaaactgtgacttagcatttcccagctgagaccttaaacctctgagtctggaatgcaccccagtcctttccttcctgtgtctgcttcgctgcgtctctctagcctctccactgtgtatatatgtgcacacatgcagacacctctacacgggagcacacacgcgagccttagagcagggtggcaggcaggccttcagtggctctcactctgagggactgcactaggattgttggtttgcgggttagcagtgtcccagctgttgcctggttgcctctgtctgctgtgattcctgagcttggaccttgggctgtggtgcaggagaactcaggctctagagtagcccagaccgtctggggcttcccttccttccttcctcccttccttcccccagtgtaaaggctgagcttgaaagctgtggttgctgagagagccagggcagcctgggccatggagaggccctctctgtagctgttaacgtaaggtggctggatcagatgtctgctacttttgatcacagttccctgaggtgggtctcacttagaagagttcctgaatgattctatttcctgtctcagtgtgccagtgaggctctggcgaggtgtgagctaacgctgctgtgtagttgggtctcttgcagaaggctagcttgttcctgtccttcccccagagctgagagtgtagaatatgctcctgttggctggcggcagtggggagggtagccagtgtggtcagtgtgctttggtggagtgagacttaagagaatcagggtcctctaagtgtccagcagaggctgtgaatttatgtgtaagatgagctcatcccaaacctcactcatgtatttgcataagagagtaagaggctaacagggaaagcagagagccatgggcctgtttgaaggtgtgaaccagtgcttcgctggggtgatggtctagagcaatgcacaggatgggaggcaggaagccgcctcttcctgctctgcccactccctgcagagccaaggttgagattaccgaccaggtagctaacatcacctttctaacttaaatttgctgccatttggtactgggttgacccagcttcagatcactctgtcatctcagtggagccagtgactcagcctttgctccgtcagttaagcttttaaagtgaggggcctaagtgctgcttcaccctacttttggggaacccactacaactggggaagggcggaagaggagagactcaggaagctgctgagggtgctggggaggcagtccccttacaggcccttgtcagttgctttctgactggaaaagggcattgatatatctttggccatgcaccatagcaagctaagaaagtgcccttcccaccccaatagcggctgcttttggtgaagaccttgccttgtgtggtatctgagcagacgagttggggactaggaggcaacttccccatcttagccctgctggatctacctcaaggttacccaagaagtccatcttgctccttcaagagccctagtccaatgaaaggaatagctgacttgttgtttaagaaggagatgttgcctgtatgcgtaaaagagctctctgtttcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccttatatgacgcacccttcaggtgaggcgtgtgtgtgcaggggccgccggggcaccccgagcgttctgctcgcagaggtagaatggcaggcaggaccagtgcttcaggctgcaaacctgactagcgaggcccatccaagtgtgcacaggagggatcgtgacggtcaggaaacatcaggctctgtgtgtgttctgctggttcagggttggcttgagaagcagcagcctcattctgggtagccgatgcatgccgaggactcagttggccttcctggttatgtatgtatgataggctggacagggcagcgtggtccgtcctgagtcctttataggctgatgtggggggtttatgcccatggccaagtttgatcgtagcttggaggagagcactggtgaacacgtgctgtgtgcggactggtctgagtgtagtgttagccccagcctgtgccctgacacaggacgcccatgatgtctcgtactgatggctgctgtgacgacacctgtcttccctgtgacttggaaagcagtggtgcttggctagtgtagtgggagggggtgtgaaggcctgctccacaagcagcaagtccaggacttgtatctgaagtggtttacgtctgactggtttgtttctcctcacccccttccctccccaaccctaccttggctgcagttgggggagaggggcagcattaaccctccagtcttactcactagcacaggagtagttgacagtcgtgtgctgcggacactaggctggcgggcagtggtgcctgtgttgtttaaatagccacttttcttttttacagtacaagcccaccaccaacagcagttgtaaggctgccctggaggaaccgaaaggccaaatcccctcctcccttctcctgcttctgccaaccggaagcacagaaaactagaacttcattgattttgttttttaaaagatacactgatttaacatctgataggaatgctaacagctcacttgcagtggaggatgttttggaccgagtagaggcatgtagggacttgtggctgttccataattccatgtgctgttgcagggtcctgcaagtacccagctctgcttgctgaaactggaagttatttattttttaatggcccttgagagtcatgaacacatcagctagcaacagaagtaacaagagtgattcttgctgctattaccgctttaaaaaaaaaaaatcaagacttggaacgcccttttactaaacttgacagaagttcagtaaattcttaccgccaaactgacggattattatttataaatcaagtttgatgaggcgatcactgtctacagtggttcaacttttaagttaagggaaaacttttactttgtagataatataaaataaaaactaaaaaaaaaaattaaaaaataaaaaaagttttaaaaactgatacccagttagtgtgtgtttatgttgactcctgctgctgtgggaggtagaccccagggaagacaggctggttgtgctgctccaatctctgaggaggtgctgaccaaatgacttttcttcttcacctttagttctggctcaaggtatctagaaaaagtcaagactgtgattccctctcgggtggattagcagttggcttcagcagaaggcaggctggccagaggtgggcagcgttcaaaagtccacatctgcaagcaggtgcagactgtaagctcacttttccttgttgatctgagttatcccagttcagttctgtgttaccagtagggggcagaagctccttctttacagaggaatctgaccaggctcaaggtgtggccacagtggctaagcacttgtctagtagcacagagccttgtccctcctcagtgtagccagggattggggaggggggggcagcacagaatgaggccaggctgcccgaaagggaggcaggcaagtgggatgggtgtgtggatgcagagatcatggttgtaatactggaaacctttttaattgacatacagcaatttgtacattgtagcaaaatttgcattattcaagaataagttactcatacagtacataaaacgaagatttgccaaataccttctgctgtaatgatagaagatgagattccctgtttctgtggtgacaatgctgcttattccggccacacactggccattagatgtgtcttcactaggaggcagtgggtgacagtttatcacttggtgtttgcaccaaggtaatattaaacaagagactgagccctcagggaaggggtgaagccttactttaagaaacttaggtaaagatagtgttgtgtggcccgagattgtaatgatccctattcctttctcctacaccatccagaaatgatcagaggcagaagatttatacagataaagccatatggattgctggtaaagtcaaggcaggttagttgacccaactctttggtgctggtgacttttagtttgtagttgagtactca

SEQ ID NO: 2 - RNA, 4505 nt, Mus musculus
  source 1..4505: mol_type = mRNA
ccgtccgcgtccgccagcccgggtcccatgcgttcgtccaccgccgccgctcagcggcccgcggcgggggaccccgagccgcgccgcccggcgggctgggccgcgcggcgctcgctcccgcggacggcgcggcgcggcgggcggggcggcgcggtggcgtatccctccgccggccctcccccgcgcggccccggcgcccctccccgcgggccgcgctcgccaccctgcgcctcagactgttttggtagcaacggccacggcgcgtcccggcccggctcccggcggctgctcggtgtctgcgggcctccccgccccttcgtcgttgtcctgctgcctctggcccccgcggccacgccggcccgcgcctgcccgcccggcgtccgcgcgtccccgccgcgctccggcgtctcctcctcggcgcgcccggcacccggctgtccccgcccggcgtgcgagccggtgtatgggccgctcaccatgtcgctgaagccgcagccgcagccgcccgcgcccgccactggccgcaagcccggcggcggcctgctctcgtcgcccggcgccgcgccggcctcggccgcggtgacctcggcttccgtggtgccggccccggccgcgccggtggcgtcttcctcggcggccgcgggcggcgggcgtcccggcctgggcagaggtcggaacagtagcaaaggactgcctcagcctacgatttcttttgatggaatctatgcaaacgtgaggatggttcatatacttacgtcagttgttggatcgaaatgtgaagtacaagtgaaaaacggaggcatatatgaaggagtttttaaaacatacagtcctaagtgtgacttggtacttgatgctgcacatgagaaaagtacagaatccagttcggggccaaaacgtgaagaaataatggagagtgttttgttcaaatgctcagacttcgttgtggtacagtttaaagatacagactccagttatgcacggagagatgcttttactgactctgctctcagcgcaaaggtgaatggtgagcacaaggagaaggacctggagccctgggatgcaggggagctcacggccagcgaggagctggagctggagaatgatgtgtctaatggatgggaccccaatgacatgtttcgatataatgaagagaattatggtgtggtgtccacatatgatagcagtttatcttcatatacggttcctttagaaagggacaactcagaagaatttcttaaacgggaggcaagggcaaaccagttagcagaagaaattgaatccagtgctcagtacaaagctcgtgtcgcccttgagaatgatgaccggagtgaggaagaaaaatacacagcagtccagagaaactgcagtgaccgggaggggcatggccccaacactagggacaataaatatattcctcctggacaaagaaacagagaagtcctatcctggggaagtgggagacagagctcaccacggatgggccagcctgggccaggctccatgccgtcaagagctgcttctcacacttcagatttcaacccgaacgctggctcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgaggccctccagacccccgtctcacccctctgctcatggttctccagctcctgtctctactatgcctaaacgcatgtcttcagaaggacccccaaggatgtctccaaaggcacagcgccaccctcggaatcacagagtctctgctgggagaggctccatgtctagtggcctagaatttgtatcccacaatcccccaagtgaagcagctgctcctccagtggcaaggaccagtcctgcagggggaacgtggtcctcagtggtcagtggggttccaaggttatctcccaaaactcacagacccaggtctcccaggcagagcagcattggaaactctcccagcgggcctgtgcttgcttctccccaagctggcatcatccctgcagaagccgtttccatgcctgttcccgccgcatctccgactcctgccagccctgcatccaacagagcactgaccccatctattgaggcaaaagattccaggcttcaagatcagaggcagaactctcctgcagggagtaaagaaaatgttaaagcaagtgaaacatcacctagcttttcaaaagctgacaacaaaggtatgtcaccagttgtttctgaacacagaaaacagattgatgacttaaagaagtttaagaatgattttaggttacagccaagctctacatctgaatctatggatcaactactaagcaaaaatagagaaggagaaaagtcacgagatttgattaaagataaaacggaagcaagtgctaaggatagtttcattgacagcagcagcagcagcagcaactgtaccagtggcagcagcaagaccaacagccctagcatctccccttccatgcttagtaatgcagagcacaagagggggcctgaggtcacatcccaaggggtgcagacttccagcccagcctgcaaacaagagaaggatgacagagaagagaagaaagacacaacagagcaggttaggaaatcgacattgaatcccaatgcaaaggagttcaaccctcgttctttctctcagccaaagccttctactaccccaacgtcacctcggcctcaagcacaacccagcccatctatggtgggtcatcagcagccagctccagtgtacactcagcctgtgtgcttcgcacccaatatgatgtatcccgtcccagtgagcccgggcgtacaacctttatacccaatacctatgacgcccatgcctgtgaaccaagccaagacatatagagcaggtaaagtaccaaatatgccccaacagcgacaagaccaacatcatcaaagcaccatgatgcacccagcctccgcggcagggccacccatcgtagccaccccgcccgcttactccactcagtacgttgcctacagccctcagcagtttcccaatcagcctttggtccagcatgtgccgcattatcagtctcagcatcctcatgtgtacagtcctgtcatacaaggtaatgccaggatgatggcaccaccagcacatgctcagcctggtttagtgtcttcttcagctgctcagttcggggctcacgagcagacgcacgccatgtatgcatgtcccaaattaccatacaacaaggagacaagcccttctttctactttgccatttccaccggctccctcgctcagcagtatgcacatcctaatgccgccctgcatccacatactccccatcctcagccttcggccactcccaccggacagcagcaaagccagcatggtggaagtcaccctgcacccagtcctgttcagcaccatcagcaccaggctgcccaggctcttcatctggccagtccacagcagcagtcggccatttatcatgcggggctggcaccaacaccaccttccatgacacctgcctctaatacacagtctccacagagcagtttcccagcagcacaacagacagtcttcaccatccacccttctcatgttcagccggcatacaccaccccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccttatatgacgcacccttcagtacaagcccaccaccaacagcagttgtaaggctgccctggaggaaccgaaaggccaaatcccctcctcccttctcctgcttctgccaaccggaagcacagaaaactagaacttcattgattttgttttttaaaagatacactgatttaacatctgataggaatgctaacagctcacttgcagtggaggatgttttggaccgagtagaggcatgtagggacttgtggctgttccataattccatgtgctgttgcagggtcctgcaagtacccagctctgcttgctgaaactggaagttatttattttttaatggcccttgagagtcatgaacacatcagctagcaacagaagtaacaagagtgattcttgctgctattaccgctttaaaaaaaaaaaatcaagacttggaacgcccttttactaaacttgacagaagttcagtaaattcttaccgccaaactgacggattattatttataaatcaagtttgatgaggcgatcactgtctacagtggttcaacttttaagttaagggaaaacttttactttgtagataatataaaataaaaactaaaaaaaaaaattaaaaaataaaaaaagttttaaaaactgaaaaaaaaaaaaaaaaaaaaaaaaaaaaaaaaaa

SEQ ID NO: 3 - RNA, 4712 nt, Homo sapiens
  source 1..4712: mol_type = mRNA
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 4 - skipped

SEQ ID NO: 5 - RNA, 863 nt, Homo sapiens
  source 1..863: mol_type = mRNA
gtctgtcggggctctctccccgccccctccggatcctgggnaagnacggnggacggggtggagacaagtgggccttggcccccgcacccctctgcgttcgtgtccgaggcggcggcgggggctcccgaactcccctgaaatcgtggggctccatgtggcctccggcagcgttccaccctcccccacctggggaagggaaggggtggggagtgcccggccccgtcccggccttcctccttcccccgccagacctctccggcgcgcgggtggtggccgatccgcattgctgttcgaggccgcagtggagaaggcgcctgtggaacatcgaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggaccctgcagcccctggatgcaggtgaactcacagccaatgagggaactggaggctttgnaaaatgacgtatctaatggatggaacccaaagatatgtttcgtttaatgaaaaaaattatggcgcaggggccaccgttgaaagcagtttatttcggatac

SEQ ID NO: 6-10 - skipped (5 entries)

SEQ ID NO: 11 - DNA, 27 bp, synthetic construct
  source 1..27: mol_type = other DNA
accaaagagtagttaatggaggtgttc

SEQ ID NO: 12 - DNA, 19 bp, synthetic construct
  source 1..19: mol_type = other DNA
agaaggtgggcgagaggaa

SEQ ID NO: 13 - DNA, 19 bp, synthetic construct
  source 1..19: mol_type = other DNA
ctggccatcgccttgccca

SEQ ID NO: 14 - DNA, 20 bp, synthetic construct
  source 1..20: mol_type = other DNA
cgtttgcatagattccatca

SEQ ID NO: 15 - DNA, 20 bp, synthetic construct
  source 1..20: mol_type = other DNA
cttcacatttcgatccaaca

SEQ ID NO: 16 - DNA, 20 bp, synthetic construct
  source 1..20: mol_type = other DNA
tcatatatgcctccgttttt

SEQ ID NO: 17 - DNA, 20 bp, synthetic construct
  source 1..20: mol_type = other DNA
gtggacaccacaccataatt

SEQ ID NO: 18 - DNA, 20 bp, synthetic construct
  source 1..20: mol_type = other DNA
ctggattcaatttcttctgc

SEQ ID NO: 19 - DNA, 20 bp, synthetic construct
  source 1..20: mol_type = other DNA
tgtactgagcactggattca

SEQ ID NO: 20 - DNA, 20 bp, synthetic construct
  source 1..20: mol_type = other DNA
gtcatcattctcaagggcga

SEQ ID NO: 21 - DNA, 20 bp, synthetic construct
  source 1..20: mol_type = other DNA
ggtcatcattctcaagggcg

SEQ ID NO: 22 - DNA, 20 bp, synthetic construct
  source 1..20: mol_type = other DNA
ttcttcctcactccggtcat

SEQ ID NO: 23 - DNA, 20 bp, synthetic construct
  source 1..20: mol_type = other DNA
ggtgatgtttcacttgcttt

SEQ ID NO: 24 - DNA, 20 bp, synthetic construct
  source 1..20: mol_type = other DNA
gtcatcaatctgttttctgt

SEQ ID NO: 25 - DNA, 20 bp, synthetic construct
  source 1..20: mol_type = other DNA
gtgacttttctccttctcta

SEQ ID NO: 26 - DNA, 20 bp, synthetic construct
  source 1..20: mol_type = other DNA
gcttccgttttatctttaat

SEQ ID NO: 27 - DNA, 20 bp, synthetic construct
  source 1..20: mol_type = other DNA
agcacttgcttccgttttat

SEQ ID NO: 28 - DNA, 20 bp, synthetic construct
  source 1..20: mol_type = other DNA
tgctgctgctgtcaatgaaa

SEQ ID NO: 29 - DNA, 20 bp, synthetic construct
  source 1..20: mol_type = other DNA
aatgtcgatttcctaacctg

SEQ ID NO: 30 - DNA, 20 bp, synthetic construct
  source 1..20: mol_type = other DNA
gaccaaaggctgattgggaa

SEQ ID NO: 31 - DNA, 20 bp, synthetic construct
  source 1..20: mol_type = other DNA
ggaccaaaggctgattggga

SEQ ID NO: 32 - DNA, 20 bp, synthetic construct
  source 1..20: mol_type = other DNA
caggactgtacacatgagga

SEQ ID NO: 33 - DNA, 20 bp, synthetic construct
  source 1..20: mol_type = other DNA
cactaaaccaggctgagcat

SEQ ID NO: 34 - DNA, 20 bp, synthetic construct
  source 1..20: mol_type = other DNA
ctgttagcattcctatcaga

SEQ ID NO: 35 - DNA, 20 bp, synthetic construct
  source 1..20: mol_type = other DNA
gtccaaaacatcctccactg

SEQ ID NO: 36 - DNA, 20 bp, synthetic construct
  source 1..20: mol_type = other DNA
tgcctctactcggtccaaaa

SEQ ID NO: 37 - DNA, 20 bp, synthetic construct
  source 1..20: mol_type = other DNA
ccctacatgcctctactcgg

SEQ ID NO: 38 - DNA, 20 bp, synthetic construct
  source 1..20: mol_type = other DNA
agccacaagtccctacatgc

SEQ ID NO: 39 - DNA, 20 bp, synthetic construct
  source 1..20: mol_type = other DNA
gttcatgactctcaagggcc

SEQ ID NO: 40 - DNA, 20 bp, synthetic construct
  source 1..20: mol_type = other DNA
acttctgtcaagtttagtaa

SEQ ID NO: 41 - DNA, 20 bp, synthetic construct
  source 1..20: mol_type = other DNA
agttttcccttaacttaaaa

SEQ ID NO: 42 - DNA, 20 bp, synthetic construct
  source 1..20: mol_type = other DNA
gacaaaatgagccagtttta

SEQ ID NO: 43 - DNA, 20 bp, synthetic construct
  source 1..20: mol_type = other DNA
aatctgtgttttccctttcg

SEQ ID NO: 44 - DNA, 20 bp, synthetic construct
  source 1..20: mol_type = other DNA
tagcaccaggtatgcccatg

SEQ ID NO: 45 - DNA, 20 bp, synthetic construct
  source 1..20: mol_type = other DNA
catggttatctatggcagca

SEQ ID NO: 46 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE guanosine 3'-phosphodiester
  modified_base 3..5: mod_base = OTHER; note = 2'-MOE thymidine 3'-phosphodiester
  modified_base 6: mod_base = OTHER; note = 2'-deoxyguanosine 3'-phosphorothioate
  modified_base 7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11: mod_base = OTHER; note = 2'-deoxyguanosine 3'-phosphorothioate
  modified_base 12: mod_base = OTHER; note = 2'-deoxyadenosine 3'-phosphorothioate
  modified_base 13..14: mod_base = OTHER; note = 2'-deoxythymidine 3'-phosphorothioate
  modified_base 15: mod_base = OTHER; note = 5-methyl-2'-deoxycytidine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18: mod_base = OTHER; note = 2'-MOE thymidine 3'-phosphorothioate
  modified_base 19: mod_base = OTHER; note = 2'-MOE 5-methylcytidine 3'-phosphorothioate
  modified_base 20: mod_base = OTHER; note = 2'-MOE adenosine
cgtttgcatagattccatca

SEQ ID NO: 47 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2..3: mod_base = OTHER; note = 2'-MOE thymidine 3'-phosphodiester
  modified_base 4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8..10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = 2'-deoxythymidine 3'-phosphorothioate
  modified_base 15: mod_base = OTHER; note = 5-methyl-2'-deoxycytidine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18: mod_base = OTHER; note = 2'-MOE adenosine 3'-phosphorothioate
  modified_base 19: mod_base = OTHER; note = 2'-MOE 5-methylcytidine 3'-phosphorothioate
  modified_base 20: mod_base = OTHER; note = 2'-MOE adenosine
cttcacatttcgatccaaca

SEQ ID NO: 48 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE 5-methylcytidine 3'-phosphodiester
  modified_base 3: mod_base = OTHER; note = 2'-MOE adenosine 3'-phosphodiester
  modified_base 4: mod_base = OTHER; note = 2'-MOE thymidine 3'-phosphodiester
  modified_base 5: mod_base = OTHER; note = 2'-MOE adenosine 3'-phosphodiester
  modified_base 6: mod_base = OTHER; note = 2'-deoxythymidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10..11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13..14: mod_base = OTHER; note = 5-methyl-2'-deoxycytidine 3'-phosphorothioate
  modified_base 15: mod_base = OTHER; note = 2'-deoxyguanosine 3'-phosphorothioate
  modified_base 16..17: mod_base = OTHER; note = 2'-MOE thymidine 3'-phosphodiester
  modified_base 18..19: mod_base = OTHER; note = 2'-MOE thymidine 3'-phosphorothioate
  modified_base 20: mod_base = OTHER; note = 2'-MOE thymidine
tcatatatgcctccgttttt

SEQ ID NO: 49 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3..4: mod_base = OTHER; note = 2'-MOE guanosine 3'-phosphodiester
  modified_base 5: mod_base = OTHER; note = 2'-MOE adenosine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8..9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyadenosine 3'-phosphorothioate
  modified_base 13..14: mod_base = OTHER; note = 5-methyl-2'-deoxycytidine 3'-phosphorothioate
  modified_base 15: mod_base = OTHER; note = 2'-deoxyadenosine 3'-phosphorothioate
  modified_base 16: mod_base = OTHER; note = 2'-MOE thymidine 3'-phosphodiester
  modified_base 17: mod_base = OTHER; note = 2'-MOE adenosine 3'-phosphodiester
  modified_base 18: mod_base = OTHER; note = 2'-MOE adenosine 3'-phosphorothioate
  modified_base 19: mod_base = OTHER; note = 2'-MOE thymidine 3'-phosphorothioate
  modified_base 20: mod_base = OTHER; note = 2'-MOE thymidine
gtggacaccacaccataatt

SEQ ID NO: 50 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE thymidine 3'-phosphodiester
  modified_base 3..4: mod_base = OTHER; note = 2'-MOE guanosine 3'-phosphodiester
  modified_base 5: mod_base = OTHER; note = 2'-MOE adenosine 3'-phosphodiester
  modified_base 6..7: mod_base = OTHER; note = 2'-deoxythymidine 3'-phosphorothioate
  modified_base 8: mod_base = OTHER; note = 5-methyl-2'-deoxycytidine 3'-phosphorothioate
  modified_base 9..10: mod_base = OTHER; note = 2'-deoxyadenosine 3'-phosphorothioate
  modified_base 11..13: mod_base = OTHER; note = 2'-deoxythymidine 3'-phosphorothioate
  modified_base 14: mod_base = OTHER; note = 5-methyl-2'-deoxycytidine 3'-phosphorothioate
  modified_base 15: mod_base = OTHER; note = 2'-deoxythymidine 3'-phosphorothioate
  modified_base 16: mod_base = OTHER; note = 2'-MOE thymidine 3'-phosphodiester
  modified_base 17: mod_base = OTHER; note = 2'-MOE 5-methylcytidine 3'-phosphodiester
  modified_base 18: mod_base = OTHER; note = 2'-MOE thymidine 3'-phosphorothioate
  modified_base 19: mod_base = OTHER; note = 2'-MOE guanosine 3'-phosphorothioate
  modified_base 20: mod_base = OTHER; note = 2'-MOE 5-methylcytidine
ctggattcaatttcttctgc

SEQ ID NO: 51 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE guanosine 3'-phosphodiester
  modified_base 3: mod_base = OTHER; note = 2'-MOE thymidine 3'-phosphodiester
  modified_base 4: mod_base = OTHER; note = 2'-MOE adenosine 3'-phosphodiester
  modified_base 5: mod_base = OTHER; note = 2'-MOE 5-methylcytidine 3'-phosphodiester
  modified_base 6: mod_base = OTHER; note = 2'-deoxythymidine 3'-phosphorothioate
  modified_base 7: mod_base = OTHER; note = 2'-deoxyguanosine 3'-phosphorothioate
  modified_base 8: mod_base = OTHER; note = 2'-deoxyadenosine 3'-phosphorothioate
  modified_base 9: mod_base = OTHER; note = 2'-deoxyguanosine 3'-phosphorothioate
  modified_base 10: mod_base = OTHER; note = 5-methyl-2'-deoxycytidine 3'-phosphorothioate
  modified_base 11: mod_base = OTHER; note = 2'-deoxyadenosine 3'-phosphorothioate
  modified_base 12: mod_base = OTHER; note = 5-methyl-2'-deoxycytidine 3'-phosphorothioate
  modified_base 13: mod_base = OTHER; note = 2'-deoxythymidine 3'-phosphorothioate
  modified_base 14..15: mod_base = OTHER; note = 2'-deoxyguanosine 3'-phosphorothioate
  modified_base 16: mod_base = OTHER; note = 2'-MOE adenosine 3'-phosphodiester
  modified_base 17: mod_base = OTHER; note = 2'-MOE thymidine 3'-phosphodiester
  modified_base 18: mod_base = OTHER; note = 2'-MOE thymidine 3'-phosphorothioate
  modified_base 19: mod_base = OTHER; note = 2'-MOE 5-methylcytidine 3'-phosphorothioate
  modified_base 20: mod_base = OTHER; note = 2'-MOE adenosine
tgtactgagcactggattca

SEQ ID NO: 52 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3: mod_base = OTHER; note = 2'-MOE 5-methylcytidine 3'-phosphodiester
  modified_base 4: mod_base = OTHER; note = 2'-MOE adenosine 3'-phosphodiester
  modified_base 5: mod_base = OTHER; note = 2'-MOE thymidine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8..9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11: mod_base = OTHER; note = 2'-deoxythymidine 3'-phosphorothioate
  modified_base 12: mod_base = OTHER; note = 5-methyl-2'-deoxycytidine 3'-phosphorothioate
  modified_base 13..14: mod_base = OTHER; note = 2'-deoxyadenosine 3'-phosphorothioate
  modified_base 15: mod_base = OTHER; note = 2'-deoxyguanosine 3'-phosphorothioate
  modified_base 16..17: mod_base = OTHER; note = 2'-MOE guanosine 3'-phosphodiester
  modified_base 18: mod_base = OTHER; note = 2'-MOE 5-methylcytidine 3'-phosphorothioate
  modified_base 19: mod_base = OTHER; note = 2'-MOE guanosine 3'-phosphorothioate
  modified_base 20: mod_base = OTHER; note = 2'-MOE adenosine
gtcatcattctcaagggcga

SEQ ID NO: 53 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE thymidine 3'-phosphodiester
  modified_base 4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6: mod_base = OTHER; note = 2'-deoxythymidine 3'-phosphorothioate
  modified_base 7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9..10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14..15: mod_base = OTHER; note = 2'-deoxyadenosine 3'-phosphorothioate
  modified_base 16..17: mod_base = OTHER; note = 2'-MOE guanosine 3'-phosphodiester
  modified_base 18: mod_base = OTHER; note = 2'-MOE guanosine 3'-phosphorothioate
  modified_base 19: mod_base = OTHER; note = 2'-MOE 5-methylcytidine 3'-phosphorothioate
  modified_base 20: mod_base = OTHER; note = 2'-MOE guanosine
ggtcatcattctcaagggcg

SEQ ID NO: 54 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE thymidine 3'-phosphodiester
  modified_base 3: mod_base = OTHER; note = 2'-MOE 5-methylcytidine 3'-phosphodiester
  modified_base 4..5: mod_base = OTHER; note = 2'-MOE thymidine 3'-phosphodiester
  modified_base 6..7: mod_base = OTHER; note = 5-methyl-2'-deoxycytidine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 5-methyl-2'-deoxycytidine 3'-phosphorothioate
  modified_base 12: mod_base = OTHER; note = 5-methyl-2'-deoxycytidine 3'-phosphorothioate
  modified_base 13..14: mod_base = OTHER; note = 5-methyl-2'-deoxycytidine 3'-phosphorothioate
  modified_base 15: mod_base = OTHER; note = 2'-deoxyguanosine 3'-phosphorothioate
  modified_base 16: mod_base = OTHER; note = 2'-MOE guanosine 3'-phosphodiester
  modified_base 17: mod_base = OTHER; note = 2'-MOE thymidine 3'-phosphodiester
  modified_base 18: mod_base = OTHER; note = 2'-MOE 5-methylcytidine 3'-phosphorothioate
  modified_base 19: mod_base = OTHER; note = 2'-MOE adenosine 3'-phosphorothioate
  modified_base 20: mod_base = OTHER; note = 2'-MOE thymidine
ttcttcctcactccggtcat

SEQ ID NO: 55 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE thymidine 3'-phosphodiester
  modified_base 4: mod_base = OTHER; note = 2'-MOE guanosine 3'-phosphodiester
  modified_base 5: mod_base = OTHER; note = 2'-MOE adenosine 3'-phosphodiester
  modified_base 6: mod_base = OTHER; note = 2'-deoxythymidine 3'-phosphorothioate
  modified_base 7: mod_base = OTHER; note = 2'-deoxyguanosine 3'-phosphorothioate
  modified_base 8..10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyadenosine 3'-phosphorothioate
  modified_base 13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-MOE guanosine 3'-phosphodiester
  modified_base 17: mod_base = OTHER; note = 2'-MOE 5-methylcytidine 3'-phosphodiester
  modified_base 18..19: mod_base = OTHER; note = 2'-MOE thymidine 3'-phosphorothioate
  modified_base 20: mod_base = OTHER; note = 2'-MOE thymidine
ggtgatgtttcacttgcttt

SEQ ID NO: 56 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3: mod_base = OTHER; note = 2'-MOE 5-methylcytidine 3'-phosphodiester
  modified_base 4: mod_base = OTHER; note = 2'-MOE adenosine 3'-phosphodiester
  modified_base 5: mod_base = OTHER; note = 2'-MOE thymidine 3'-phosphodiester
  modified_base 6: mod_base = OTHER; note = 5-methyl-2'-deoxycytidine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11: mod_base = OTHER; note = 2'-deoxythymidine 3'-phosphorothioate
  modified_base 12: mod_base = OTHER; note = 2'-deoxyguanosine 3'-phosphorothioate
  modified_base 13..15: mod_base = OTHER; note = 2'-deoxythymidine 3'-phosphorothioate
  modified_base 16: mod_base = OTHER; note = 2'-MOE thymidine 3'-phosphodiester
  modified_base 17: mod_base = OTHER; note = 2'-MOE 5-methylcytidine 3'-phosphodiester
  modified_base 18: mod_base = OTHER; note = 2'-MOE thymidine 3'-phosphorothioate
  modified_base 19: mod_base = OTHER; note = 2'-MOE guanosine 3'-phosphorothioate
  modified_base 20: mod_base = OTHER; note = 2'-MOE thymidine
gtcatcaatctgttttctgt

SEQ ID NO: 57 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3: mod_base = OTHER; note = 2'-MOE guanosine 3'-phosphodiester
  modified_base 4: mod_base = OTHER; note = 2'-MOE adenosine 3'-phosphodiester
  modified_base 5: mod_base = OTHER; note = 2'-MOE 5-methylcytidine 3'-phosphodiester
  modified_base 6..9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14..15: mod_base = OTHER; note = 2'-deoxythymidine 3'-phosphorothioate
  modified_base 16: mod_base = OTHER; note = 2'-MOE 5-methylcytidine 3'-phosphodiester
  modified_base 17: mod_base = OTHER; note = 2'-MOE thymidine 3'-phosphodiester
  modified_base 18: mod_base = OTHER; note = 2'-MOE 5-methylcytidine 3'-phosphorothioate
  modified_base 19: mod_base = OTHER; note = 2'-MOE thymidine 3'-phosphorothioate
  modified_base 20: mod_base = OTHER; note = 2'-MOE adenosine
gtgacttttctccttctcta

SEQ ID NO: 58 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE 5-methylcytidine 3'-phosphodiester
  modified_base 3..4: mod_base = OTHER; note = 2'-MOE thymidine 3'-phosphodiester
  modified_base 5: mod_base = OTHER; note = 2'-MOE 5-methylcytidine 3'-phosphodiester
  modified_base 6: mod_base = OTHER; note = 5-methyl-2'-deoxycytidine 3'-phosphorothioate
  modified_base 7: mod_base = OTHER; note = 2'-deoxyguanosine 3'-phosphorothioate
  modified_base 8..11: mod_base = OTHER; note = 2'-deoxythymidine 3'-phosphorothioate
  modified_base 12: mod_base = OTHER; note = 2'-deoxyadenosine 3'-phosphorothioate
  modified_base 13: mod_base = OTHER; note = 2'-deoxythymidine 3'-phosphorothioate
  modified_base 14: mod_base = OTHER; note = 5-methyl-2'-deoxycytidine 3'-phosphorothioate
  modified_base 15: mod_base = OTHER; note = 2'-deoxythymidine 3'-phosphorothioate
  modified_base 16..17: mod_base = OTHER; note = 2'-MOE thymidine 3'-phosphodiester
  modified_base 18..19: mod_base = OTHER; note = 2'-MOE adenosine 3'-phosphorothioate
  modified_base 20: mod_base = OTHER; note = 2'-MOE thymidine
gcttccgttttatctttaat

SEQ ID NO: 59 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE guanosine 3'-phosphodiester
  modified_base 3: mod_base = OTHER; note = 2'-MOE 5-methylcytidine 3'-phosphodiester
  modified_base 4: mod_base = OTHER; note = 2'-MOE adenosine 3'-phosphodiester
  modified_base 5: mod_base = OTHER; note = 2'-MOE 5-methylcytidine 3'-phosphodiester
  modified_base 6..7: mod_base = OTHER; note = 2'-deoxythymidine 3'-phosphorothioate
  modified_base 8: mod_base = OTHER; note = 2'-deoxyguanosine 3'-phosphorothioate
  modified_base 9: mod_base = OTHER; note = 5-methyl-2'-deoxycytidine 3'-phosphorothioate
  modified_base 10..11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxyguanosine 3'-phosphorothioate
  modified_base 15: mod_base = OTHER; note = 2'-deoxythymidine 3'-phosphorothioate
  modified_base 16..17: mod_base = OTHER; note = 2'-MOE thymidine 3'-phosphodiester
  modified_base 18: mod_base = OTHER; note = 2'-MOE thymidine 3'-phosphorothioate
  modified_base 19: mod_base = OTHER; note = 2'-MOE adenosine 3'-phosphorothioate
  modified_base 20: mod_base = OTHER; note = 2'-MOE thymidine
agcacttgcttccgttttat

SEQ ID NO: 60 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE guanosine 3'-phosphodiester
  modified_base 3: mod_base = OTHER; note = 2'-MOE 5-methylcytidine 3'-phosphodiester
  modified_base 4: mod_base = OTHER; note = 2'-MOE thymidine 3'-phosphodiester
  modified_base 5: mod_base = OTHER; note = 2'-MOE guanosine 3'-phosphodiester
  modified_base 6: mod_base = OTHER; note = 5-methyl-2'-deoxycytidine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxythymidine 3'-phosphorothioate
  modified_base 11: mod_base = OTHER; note = 2'-deoxyguanosine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14..15: mod_base = OTHER; note = 2'-deoxyadenosine 3'-phosphorothioate
  modified_base 16: mod_base = OTHER; note = 2'-MOE thymidine 3'-phosphodiester
  modified_base 17: mod_base = OTHER; note = 2'-MOE guanosine 3'-phosphodiester
  modified_base 18..19: mod_base = OTHER; note = 2'-MOE adenosine 3'-phosphorothioate
  modified_base 20: mod_base = OTHER; note = 2'-MOE adenosine
tgctgctgctgtcaatgaaa

SEQ ID NO: 61 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE adenosine 3'-phosphodiester
  modified_base 3: mod_base = OTHER; note = 2'-MOE thymidine 3'-phosphodiester
  modified_base 4: mod_base = OTHER; note = 2'-MOE guanosine 3'-phosphodiester
  modified_base 5: mod_base = OTHER; note = 2'-MOE thymidine 3'-phosphodiester
  modified_base 6: mod_base = OTHER; note = 5-methyl-2'-deoxycytidine 3'-phosphorothioate
  modified_base 7: mod_base = OTHER; note = 2'-deoxyguanosine 3'-phosphorothioate
  modified_base 8: mod_base = OTHER; note = 2'-deoxyadenosine 3'-phosphorothioate
  modified_base 9..11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-MOE adenosine 3'-phosphodiester
  modified_base 17: mod_base = OTHER; note = 2'-MOE 5-methylcytidine 3'-phosphodiester
  modified_base 18: mod_base = OTHER; note = 2'-MOE 5-methylcytidine 3'-phosphorothioate
  modified_base 19: mod_base = OTHER; note = 2'-MOE thymidine 3'-phosphorothioate
  modified_base 20: mod_base = OTHER; note = 2'-MOE guanosine
aatgtcgatttcctaacctg

SEQ ID NO: 62 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE adenosine 3'-phosphodiester
  modified_base 3..4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6..7: mod_base = OTHER; note = 2'-deoxyadenosine 3'-phosphorothioate
  modified_base 8..9: mod_base = OTHER; note = 2'-deoxyguanosine 3'-phosphorothioate
  modified_base 10: mod_base = OTHER; note = 5-methyl-2'-deoxycytidine 3'-phosphorothioate
  modified_base 11: mod_base = OTHER; note = 2'-deoxythymidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14..15: mod_base = OTHER; note = 2'-deoxythymidine 3'-phosphorothioate
  modified_base 16..17: mod_base = OTHER; note = 2'-MOE guanosine 3'-phosphodiester
  modified_base 18: mod_base = OTHER; note = 2'-MOE guanosine 3'-phosphorothioate
  modified_base 19: mod_base = OTHER; note = 2'-MOE adenosine 3'-phosphorothioate
  modified_base 20: mod_base = OTHER; note = 2'-MOE adenosine
gaccaaaggctgattgggaa

SEQ ID NO: 63 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE guanosine 3'-phosphodiester
  modified_base 3: mod_base = OTHER; note = 2'-MOE adenosine 3'-phosphodiester
  modified_base 4..5: mod_base = OTHER; note = 2'-MOE 5-methylcytidine 3'-phosphodiester
  modified_base 6..8: mod_base = OTHER; note = 2'-deoxyadenosine 3'-phosphorothioate
  modified_base 9..10: mod_base = OTHER; note = 2'-deoxyguanosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13: mod_base = OTHER; note = 2'-deoxyguanosine 3'-phosphorothioate
  modified_base 14: mod_base = OTHER; note = 2'-deoxyadenosine 3'-phosphorothioate
  modified_base 15: mod_base = OTHER; note = 2'-deoxythymidine 3'-phosphorothioate
  modified_base 16: mod_base = OTHER; note = 2'-MOE thymidine 3'-phosphodiester
  modified_base 17: mod_base = OTHER; note = 2'-MOE guanosine 3'-phosphodiester
  modified_base 18..19: mod_base = OTHER; note = 2'-MOE guanosine 3'-phosphorothioate
  modified_base 20: mod_base = OTHER; note = 2'-MOE adenosine
ggaccaaaggctgattggga

SEQ ID NO: 64 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE adenosine 3'-phosphodiester
  modified_base 3..4: mod_base = OTHER; note = 2'-MOE guanosine 3'-phosphodiester
  modified_base 5: mod_base = OTHER; note = 2'-MOE adenosine 3'-phosphodiester
  modified_base 6: mod_base = OTHER; note = 5-methyl-2'-deoxycytidine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyadenosine 3'-phosphorothioate
  modified_base 13: mod_base = OTHER; note = 5-methyl-2'-deoxycytidine 3'-phosphorothioate
  modified_base 14: mod_base = OTHER; note = 2'-deoxyadenosine 3'-phosphorothioate
  modified_base 15: mod_base = OTHER; note = 2'-deoxythymidine 3'-phosphorothioate
  modified_base 16: mod_base = OTHER; note = 2'-MOE guanosine 3'-phosphodiester
  modified_base 17: mod_base = OTHER; note = 2'-MOE adenosine 3'-phosphodiester
  modified_base 18..19: mod_base = OTHER; note = 2'-MOE guanosine 3'-phosphorothioate
  modified_base 20: mod_base = OTHER; note = 2'-MOE adenosine
caggactgtacacatgagga

SEQ ID NO: 65 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE adenosine 3'-phosphodiester
  modified_base 3: mod_base = OTHER; note = 2'-MOE 5-methylcytidine 3'-phosphodiester
  modified_base 4: mod_base = OTHER; note = 2'-MOE thymidine 3'-phosphodiester
  modified_base 5: mod_base = OTHER; note = 2'-MOE adenosine 3'-phosphodiester
  modified_base 6..7: mod_base = OTHER; note = 2'-deoxyadenosine 3'-phosphorothioate
  modified_base 8..9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11..12: mod_base = OTHER; note = 2'-deoxyguanosine 3'-phosphorothioate
  modified_base 13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyguanosine 3'-phosphorothioate
  modified_base 16: mod_base = OTHER; note = 2'-MOE adenosine 3'-phosphodiester
  modified_base 17: mod_base = OTHER; note = 2'-MOE guanosine 3'-phosphodiester
  modified_base 18: mod_base = OTHER; note = 2'-MOE 5-methylcytidine 3'-phosphorothioate
  modified_base 19: mod_base = OTHER; note = 2'-MOE adenosine 3'-phosphorothioate
  modified_base 20: mod_base = OTHER; note = 2'-MOE thymidine
cactaaaccaggctgagcat

SEQ ID NO: 66 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE thymidine 3'-phosphodiester
  modified_base 3: mod_base = OTHER; note = 2'-MOE guanosine 3'-phosphodiester
  modified_base 4..5: mod_base = OTHER; note = 2'-MOE thymidine 3'-phosphodiester
  modified_base 6: mod_base = OTHER; note = 2'-deoxyadenosine 3'-phosphorothioate
  modified_base 7: mod_base = OTHER; note = 2'-deoxyguanosine 3'-phosphorothioate
  modified_base 8: mod_base = OTHER; note = 5-methyl-2'-deoxycytidine 3'-phosphorothioate
  modified_base 9: mod_base = OTHER; note = 2'-deoxyadenosine 3'-phosphorothioate
  modified_base 10..11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-MOE thymidine 3'-phosphodiester
  modified_base 17: mod_base = OTHER; note = 2'-MOE 5-methylcytidine 3'-phosphodiester
  modified_base 18: mod_base = OTHER; note = 2'-MOE adenosine 3'-phosphorothioate
  modified_base 19: mod_base = OTHER; note = 2'-MOE guanosine 3'-phosphorothioate
  modified_base 20: mod_base = OTHER; note = 2'-MOE adenosine
ctgttagcattcctatcaga

SEQ ID NO: 67 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE thymidine 3'-phosphodiester
  modified_base 3..4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6..8: mod_base = OTHER; note = 2'-deoxyadenosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyadenosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 5-methyl-2'-deoxycytidine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18: mod_base = OTHER; note = 2'-MOE 5-methylcytidine 3'-phosphorothioate
  modified_base 19: mod_base = OTHER; note = 2'-MOE thymidine 3'-phosphorothioate
  modified_base 20: mod_base = OTHER; note = 2'-MOE guanosine
gtccaaaacatcctccactg

SEQ ID NO: 68 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE guanosine 3'-phosphodiester
  modified_base 3..4: mod_base = OTHER; note = 2'-MOE 5-methylcytidine 3'-phosphodiester
  modified_base 5: mod_base = OTHER; note = 2'-MOE thymidine 3'-phosphodiester
  modified_base 6: mod_base = OTHER; note = 5-methyl-2'-deoxycytidine 3'-phosphorothioate
  modified_base 7: mod_base = OTHER; note = 2'-deoxythymidine 3'-phosphorothioate
  modified_base 8: mod_base = OTHER; note = 2'-deoxyadenosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12..13: mod_base = OTHER; note = 2'-deoxyguanosine 3'-phosphorothioate
  modified_base 14: mod_base = OTHER; note = 2'-deoxythymidine 3'-phosphorothioate
  modified_base 15: mod_base = OTHER; note = 5-methyl-2'-deoxycytidine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18..19: mod_base = OTHER; note = 2'-MOE adenosine 3'-phosphorothioate
  modified_base 20: mod_base = OTHER; note = 2'-MOE adenosine
tgcctctactcggtccaaaa

SEQ ID NO: 69 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2..3: mod_base = OTHER; note = 2'-MOE 5-methylcytidine 3'-phosphodiester
  modified_base 4: mod_base = OTHER; note = 2'-MOE thymidine 3'-phosphodiester
  modified_base 5: mod_base = OTHER; note = 2'-MOE adenosine 3'-phosphodiester
  modified_base 6: mod_base = OTHER; note = 5-methyl-2'-deoxycytidine 3'-phosphorothioate
  modified_base 7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10..11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-MOE 5-methylcytidine 3'-phosphodiester
  modified_base 17: mod_base = OTHER; note = 2'-MOE thymidine 3'-phosphodiester
  modified_base 18: mod_base = OTHER; note = 2'-MOE 5-methylcytidine 3'-phosphorothioate
  modified_base 19: mod_base = OTHER; note = 2'-MOE guanosine 3'-phosphorothioate
  modified_base 20: mod_base = OTHER; note = 2'-MOE guanosine
ccctacatgcctctactcgg

SEQ ID NO: 70 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE guanosine 3'-phosphodiester
  modified_base 3..4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6: mod_base = OTHER; note = 5-methyl-2'-deoxycytidine 3'-phosphorothioate
  modified_base 7..8: mod_base = OTHER; note = 2'-deoxyadenosine 3'-phosphorothioate
  modified_base 9: mod_base = OTHER; note = 2'-deoxyguanosine 3'-phosphorothioate
  modified_base 10: mod_base = OTHER; note = 2'-deoxythymidine 3'-phosphorothioate
  modified_base 11..13: mod_base = OTHER; note = 5-methyl-2'-deoxycytidine 3'-phosphorothioate
  modified_base 14: mod_base = OTHER; note = 2'-deoxythymidine 3'-phosphorothioate
  modified_base 15: mod_base = OTHER; note = 2'-deoxyadenosine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18: mod_base = OTHER; note = 2'-MOE thymidine 3'-phosphorothioate
  modified_base 19: mod_base = OTHER; note = 2'-MOE guanosine 3'-phosphorothioate
  modified_base 20: mod_base = OTHER; note = 2'-MOE 5-methylcytidine
agccacaagtccctacatgc

SEQ ID NO: 71 - DNA, 20 bp, synthetic construct
  source 1..20: mol_type = other DNA
  modified_base 1: mod_base = OTHER; note = 2'-MOE guanosine 3'-phosphorothioate
  modified_base 2..3: mod_base = OTHER; note = 2'-MOE thymidine 3'-phosphodiester
  modified_base 4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6: mod_base = OTHER; note = 2'-deoxythymidine 3'-phosphorothioate
  modified_base 7: mod_base = OTHER; note = 2'-deoxyguanosine 3'-phosphorothioate
  modified_base 8: mod_base = OTHER; note = 2'-deoxyadenosine 3'-phosphorothioate
  modified_base 9: mod_base = OTHER; note = 5-methyl-2'-deoxycytidine 3'-phosphorothioate
  modified_base 10: mod_base = OTHER; note = 2'-deoxythymidine 3'-phosphorothioate
  modified_base 11: mod_base = OTHER; note = 5-methyl-2'-deoxycytidine 3'-phosphorothioate
  modified_base 12: mod_base = OTHER; note = 2'-deoxythymidine 3'-phosphorothioate
  modified_base 13: mod_base = OTHER; note = 5-methyl-2'-deoxycytidine 3'-phosphorothioate
  modified_base 14..15: mod_base = OTHER; note = 2'-deoxyadenosine 3'-phosphorothioate
  modified_base 16..17: mod_base = OTHER; note = 2'-MOE guanosine 3'-phosphodiester
  modified_base 18: mod_base = OTHER; note = 2'-MOE guanosine 3'-phosphorothioate
  modified_base 19: mod_base = OTHER; note = 2'-MOE 5-methylcytidine 3'-phosphorothioate
  modified_base 20: mod_base = OTHER; note = 2'-MOE 5-methylcytidine
gttcatgactctcaagggcc

SEQ ID NO: 72 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE 5-methylcytidine 3'-phosphodiester
  modified_base 3..4: mod_base = OTHER; note = 2'-MOE thymidine 3'-phosphodiester
  modified_base 5: mod_base = OTHER; note = 2'-MOE 5-methylcytidine 3'-phosphodiester
  modified_base 6: mod_base = OTHER; note = 2'-deoxythymidine 3'-phosphorothioate
  modified_base 7: mod_base = OTHER; note = 2'-deoxyguanosine 3'-phosphorothioate
  modified_base 8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10..11: mod_base = OTHER; note = 2'-deoxyadenosine 3'-phosphorothioate
  modified_base 12: mod_base = OTHER; note = 2'-deoxyguanosine 3'-phosphorothioate
  modified_base 13..15: mod_base = OTHER; note = 2'-deoxythymidine 3'-phosphorothioate
  modified_base 16: mod_base = OTHER; note = 2'-MOE adenosine 3'-phosphodiester
  modified_base 17: mod_base = OTHER; note = 2'-MOE guanosine 3'-phosphodiester
  modified_base 18: mod_base = OTHER; note = 2'-MOE thymidine 3'-phosphorothioate
  modified_base 19: mod_base = OTHER; note = 2'-MOE adenosine 3'-phosphorothioate
  modified_base 20: mod_base = OTHER; note = 2'-MOE adenosine
acttctgtcaagtttagtaa

SEQ ID NO: 73 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE guanosine 3'-phosphodiester
  modified_base 3..5: mod_base = OTHER; note = 2'-MOE thymidine 3'-phosphodiester
  modified_base 6: mod_base = OTHER; note = 2'-deoxythymidine 3'-phosphorothioate
  modified_base 7..9: mod_base = OTHER; note = 5-methyl-2'-deoxycytidine 3'-phosphorothioate
  modified_base 10..11: mod_base = OTHER; note = 2'-deoxythymidine 3'-phosphorothioate
  modified_base 12..13: mod_base = OTHER; note = 2'-deoxyadenosine 3'-phosphorothioate
  modified_base 14: mod_base = OTHER; note = 5-methyl-2'-deoxycytidine 3'-phosphorothioate
  modified_base 15: mod_base = OTHER; note = 2'-deoxythymidine 3'-phosphorothioate
  modified_base 16: mod_base = OTHER; note = 2'-MOE thymidine 3'-phosphodiester
  modified_base 17: mod_base = OTHER; note = 2'-MOE adenosine 3'-phosphodiester
  modified_base 18..19: mod_base = OTHER; note = 2'-MOE adenosine 3'-phosphorothioate
  modified_base 20: mod_base = OTHER; note = 2'-MOE adenosine
agttttcccttaacttaaaa

SEQ ID NO: 74 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE adenosine 3'-phosphodiester
  modified_base 3: mod_base = OTHER; note = 2'-MOE 5-methylcytidine 3'-phosphodiester
  modified_base 4..5: mod_base = OTHER; note = 2'-MOE adenosine 3'-phosphodiester
  modified_base 6..7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 2'-deoxyadenosine 3'-phosphorothioate
  modified_base 11: mod_base = OTHER; note = 2'-deoxyguanosine 3'-phosphorothioate
  modified_base 12..13: mod_base = OTHER; note = 5-methyl-2'-deoxycytidine 3'-phosphorothioate
  modified_base 14: mod_base = OTHER; note = 2'-deoxyadenosine 3'-phosphorothioate
  modified_base 15: mod_base = OTHER; note = 2'-deoxyguanosine 3'-phosphorothioate
  modified_base 16..17: mod_base = OTHER; note = 2'-MOE thymidine 3'-phosphodiester
  modified_base 18..19: mod_base = OTHER; note = 2'-MOE thymidine 3'-phosphorothioate
  modified_base 20: mod_base = OTHER; note = 2'-MOE adenosine
gacaaaatgagccagtttta

SEQ ID NO: 75 - DNA, 20 bp, synthetic construct
  source 1..20: mol_type = other DNA
  modified_base 1: mod_base = OTHER; note = 2'-MOE adenosine 3'-phosphorothioate
  modified_base 2: mod_base = OTHER; note = 2'-MOE adenosine 3'-phosphodiester
  modified_base 3: mod_base = OTHER; note = 2'-MOE thymidine 3'-phosphodiester
  modified_base 4: mod_base = OTHER; note = 2'-MOE 5-methylcytidine 3'-phosphodiester
  modified_base 5: mod_base = OTHER; note = 2'-MOE thymidine 3'-phosphodiester
  modified_base 6: mod_base = OTHER; note = 2'-deoxyguanosine 3'-phosphorothioate
  modified_base 7: mod_base = OTHER; note = 2'-deoxythymidine 3'-phosphorothioate
  modified_base 8: mod_base = OTHER; note = 2'-deoxyguanosine 3'-phosphorothioate
  modified_base 9..12: mod_base = OTHER; note = 2'-deoxythymidine 3'-phosphorothioate
  modified_base 13..15: mod_base = OTHER; note = 5-methyl-2'-deoxycytidine 3'-phosphorothioate
  modified_base 16..17: mod_base = OTHER; note = 2'-MOE thymidine 3'-phosphodiester
  modified_base 18: mod_base = OTHER; note = 2'-MOE thymidine 3'-phosphorothioate
  modified_base 19: mod_base = OTHER; note = 2'-MOE 5-methylcytidine 3'-phosphorothioate
  modified_base 20: mod_base = OTHER; note = 2'-MOE guanosine
aatctgtgttttccctttcg

SEQ ID NO: 76 - DNA, 20 bp, synthetic construct
  source 1..20: mol_type = other DNA
  modified_base 1: mod_base = OTHER; note = 2'-MOE thymidine 3'-phosphorothioate
  modified_base 2: mod_base = OTHER; note = 2'-MOE adenosine 3'-phosphodiester
  modified_base 3: mod_base = OTHER; note = 2'-MOE guanosine 3'-phosphodiester
  modified_base 4: mod_base = OTHER; note = 2'-MOE 5-methylcytidine 3'-phosphodiester
  modified_base 5: mod_base = OTHER; note = 2'-MOE adenosine 3'-phosphodiester
  modified_base 6..7: mod_base = OTHER; note = 5-methyl-2'-deoxycytidine 3'-phosphorothioate
  modified_base 8: mod_base = OTHER; note = 2'-deoxyadenosine 3'-phosphorothioate
  modified_base 9..10: mod_base = OTHER; note = 2'-deoxyguanosine 3'-phosphorothioate
  modified_base 11: mod_base = OTHER; note = 2'-deoxythymidine 3'-phosphorothioate
  modified_base 12: mod_base = OTHER; note = 2'-deoxyadenosine 3'-phosphorothioate
  modified_base 13: mod_base = OTHER; note = 2'-deoxythymidine 3'-phosphorothioate
  modified_base 14: mod_base = OTHER; note = 2'-deoxyguanosine 3'-phosphorothioate
  modified_base 15: mod_base = OTHER; note = 5-methyl-2'-deoxycytidine 3'-phosphorothioate
  modified_base 16..17: mod_base = OTHER; note = 2'-MOE 5-methylcytidine 3'-phosphodiester
  modified_base 18: mod_base = OTHER; note = 2'-MOE adenosine 3'-phosphorothioate
  modified_base 19: mod_base = OTHER; note = 2'-MOE thymidine 3'-phosphorothioate
  modified_base 20: mod_base = OTHER; note = 2'-MOE guanosine
tagcaccaggtatgcccatg

SEQ ID NO: 77 - DNA, 20 bp, synthetic construct
  source 1..20: mol_type = other DNA
  modified_base 1: mod_base = OTHER; note = 2'-MOE 5-methylcytidine 3'-phosphorothioate
  modified_base 2: mod_base = OTHER; note = 2'-MOE adenosine 3'-phosphodiester
  modified_base 3: mod_base = OTHER; note = 2'-MOE thymidine 3'-phosphodiester
  modified_base 4..5: mod_base = OTHER; note = 2'-MOE guanosine 3'-phosphodiester
  modified_base 6..7: mod_base = OTHER; note = 2'-deoxythymidine 3'-phosphorothioate
  modified_base 8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 5-methyl-2'-deoxycytidine 3'-phosphorothioate
  modified_base 11: mod_base = OTHER; note = 2'-deoxythymidine 3'-phosphorothioate
  modified_base 12: mod_base = OTHER; note = 2'-deoxyadenosine 3'-phosphorothioate
  modified_base 13: mod_base = OTHER; note = 2'-deoxythymidine 3'-phosphorothioate
  modified_base 14..15: mod_base = OTHER; note = 2'-deoxyguanosine 3'-phosphorothioate
  modified_base 16: mod_base = OTHER; note = 2'-MOE 5-methylcytidine 3'-phosphodiester
  modified_base 17: mod_base = OTHER; note = 2'-MOE adenosine 3'-phosphodiester
  modified_base 18: mod_base = OTHER; note = 2'-MOE guanosine 3'-phosphorothioate
  modified_base 19: mod_base = OTHER; note = 2'-MOE 5-methylcytidine 3'-phosphorothioate
  modified_base 20: mod_base = OTHER; note = 2'-MOE adenosine
catggttatctatggcagca